SEQUENCE LISTING (198 sequences)

SEQ ID NO: 1 - protein, 126 aa, Bacillus cereus
MACECSSTVLTCCSDNSSNFVQDKVCNPWSSAEASTFTVYANNVNQNIVGTGYLTYDVGPGVSPANQITVTVLDSGGGTIQTFLVNEGTSISFTFRRFNIIQITTPATPIGTYQGEFCITTRYLMA

SEQ ID NO: 2 - protein, 125 aa, Bacillus mycoides
MSCNCSVTALTCCPDDSSNYVQDKVCNPWSSIENLTFTVYGNNVNQNIVGTGYITYDVGPGASPANDITVTVLDSGGGPIQTFTMVEGTSIAFTFRRFNTIQITTPATAGTYQGEFCITTRYAMS

SEQ ID NO: 3 - protein, 126 aa, Bacillus cytotoxicus
MACNCSVTALTCCPDESNNFVQDKVCNPWSSAEASTFTVYGNNINQNIVGTGYLTYDVGPGVSPANAITVNVLDSGGGIIQTFTVTEGMSVAFTFRRFDVIQIVTPATPAGTYQGEFCITTRYVMS

SEQ ID NO: 4 - protein, 125 aa, Bacillus luti
MACECSSTVLTCCSDNSSNFVQDKVCNPWSSAEDSTFTVYANNVNQNIIGTGYLTYNVGPGVSPANQITVTVLDSGGGPIQTFLVNEGTSISFTFRRFNIIQITTPVTPVGTYQGEFCITTRYLM

SEQ ID NO: 5 - protein, 126 aa, Bacillus cereus
MACECSSTVLTCCSDNSSNFVQDKVCNPRSSAEASTFTVYANNVNQNIVGTGYLTYDVGPGVSPANQITVTVLDSGGGTIQTFLVNEGTSISFTFRRFNIIQITTPATPIGTYQGEFCITTRYLMA

SEQ ID NO: 6 - protein, 126 aa, Bacillus tropicus; identical to SEQ ID NO: 1

SEQ ID NO: 7 - protein, 126 aa, Bacillus wiedmannii
MACECSGTVLTCCSDNSSNFVQDKVCNSWSSAAASTFTVYANNVNQNIVGTGYLTYDVGPGVSPANQITVAVLDSGGGTIQSFTVSEGTSIAFTFRRFNIIQITTPATPLGTYQGEFCITTRYLIS

SEQ ID NO: 8 - protein, 117 aa, Bacillus cereus
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGTAAAETGEFCMTIRYTLS

SEQ ID NO: 9 - protein, 120 aa, Bacillus cereus
MGNCGTNLSCCANAQKTIVQDKVCIDWTITTIAAPGQIIYADNISQDIYASGYLKVETGTGPVTITFYSGGTGGTAVETIIVASGSSASFTVRRFDTVAILGTTLGETGEFCMTIRYSLS

SEQ ID NO: 10 - protein, 116 aa, Bacillus cytotoxicus
MKNYSANLSCCANSQKNIVQDKVCINWTATAAPAIIYADNIAQDIYASGYLKVDTGTGPVTLVFYSGGIGGTAVETITVATGSGASFTVRRFDTVTILGTAAAETGEFCMTIRYTL

SEQ ID NO: 11 - protein, 117 aa, Bacillus cereus; identical to SEQ ID NO: 8

SEQ ID NO: 12 - protein, 117 aa, Bacillus wiedmannii
MGNCSTNLSCCANSQKTIVQDKVCIDWTASATAEVIYADNISQDIYASGYLKVDTGTGPVTLVFYSGGIAGTALETIVVATGSSASFTVRRFDTVTILGTAAAETGEFCMTIRYSLS

SEQ ID NO: 13 - protein, 117 aa, Bacillus luti
MGNCSTNLSCCANGQKTIVQDKVCIDWTAAVTAAIIYADNISQDIYASGYLKVDTGTGPVTINFYSGGTGGTIVETITVASGSSASFTVRRFDTVTIIGTAAAETGEFCMTIRYTLS

SEQ ID NO: 14 - protein, 117 aa, Bacillus tropicus
MGNCSTNLSCCANGQKTIVQDKVCIDWTAAATAAIIYADNISQDIYASGYLKVDTGTGPVTIVFYSGGVTGTALETIVVATGSSASFTVRRFDTVTILGTAAGETGEFCMTIRYTLS

SEQ ID NO: 15 - protein, 155 aa, Bacillus cereus
MKPHKNIGCFAPLSIICQPTCPCPPPILPPERGDAELVTNEFAGDILISNDFIPISQKQLKQTNTTVNIWKNDGIVSLSGTISIYNNRNSTNALSIQIISSTTNTFTALPGNTISYTGFDLQSVSVIDIPSDPSIYIEGRYCFQLTYCKSKRDCL

SEQ ID NO: 16 - protein, 150 aa, Bacillus cytotoxicus
MQKHKKIGCFAPLSIICEVPCPPPPPISNAEFVTNEFAGNFLITNDTFLPETNQLKQSDSTFELWKGDGLSSLSGSISVYNNRNSTTAITVQIVGETTNTLTALPGNTVSYTGSGLQSVSLINIPSDSSVYIEGRYCCQITYCKFKSDCM

SEQ ID NO: 17 - protein, 155 aa, Bacillus cereus
LKPHKNIGCFAPLSIICQPTCPCPPPISPPERGDAELVTNEFAGDILISNDFIPISQKQLKQTNTTVNIWKNDGIISLSGTISIYNNRNSTNALSIQIISSTTNTFTVLPGNTISYTGFDLQSVSVIDIPSDPSIYIEGRYCFQLTYCKSKRDCL

SEQ ID NO: 18 - protein, 155 aa, Bacillus wiedmannii
LKPHKNIGCFAPLSIICQPICPCPPPIPPPERCDAELITNEFAGDILISNDFIPISQKQLKQTYSTVTIWKNGGIVSLSGTISIYNNRNSTSALSVQIIGSTTNIFTVLPGNTISYTGFDLLSVSIIDIPSDPSIYIEGRYCFQLTYCKSKLDCL

SEQ ID NO: 19 - protein, 155 aa, Bacillus wiedmannii; identical to SEQ ID NO: 18

SEQ ID NO: 20 - protein, 155 aa, Bacillus luti
LKPHKNIGCFAPLSIICQPICPCPPPIPPPEQCDAELITNEFAGNIFISNDFISVAQRQSKQIYPTLTIWKDDGSISLSGTISVYNSKNSTDALTVQIVSNNTNTFTVLPGNTISYTGSNLQSVSIIDIPTDPSTYIEGRYCFQLTYCKSKRDCL

SEQ ID NO: 21 - protein, 126 aa, Bacillus cytotoxicus
MACNCSVTALTCCPDESNNFVQDKVCNPWSSSEASTFTVYGNNINQNIVGTGYLTYDVGPGVSPANAITVNVLDSGGGIIQTFTVTEGMSVAFTFRRFDVIQIVTPATPVGTYQGEFCITTRYVMS

SEQ ID NO: 22 - protein, 122 aa, Bacillus wiedmannii
MSCECSGIALTCCPDKNYVQDKVCSPWSATVVATAIDNVLYTNNINQNVVGTGFVKYDVGPGPITVEVLDSAGTVLDTQTLNPGTSIGFTYRRFDIIQVVLPATPAGTYQGEFCITTRYPLS

SEQ ID NO: 23 - protein, 119 aa, Bacillus manliponensis
MSCECTSTVLSCCPDKTFVQDKVCSPWSGTVVAADVTVILYTNNINQTVVGTGFIKYDVGPTDITVQVLDSTATIIDTQTLSPGSSLGFTYRQFNTIQVILPLATPGVYQGEFCITSRY

SEQ ID NO: 24 - protein, 122 aa, Bacillus cereus
MSCECSGAALTCCPDKNYVQDKVCSPWSGTVVATAITNVLYNNNINQNVIGTGFVRYDVGPAAITLTVLDSAGNTLDTQTLNPGTSIAFTYRRFETIEVTLPAATAGTYQGEFCITTRYPLS

SEQ ID NO: 25 - protein, 122 aa, Bacillus cereus
MSCECSGSALTCCPDKNYVQDKVCSPWSGTVVATAITNVLYNNNINQNMIGTGFVRYDVGPAPITLTVLDAAGATIDTQTLNPGTSIAFTYRRFVTIEVTLPAATAGTYQGEFCITTRYPLS

SEQ ID NO: 26 - protein, 122 aa, Bacillus anthracis
MSCECSGTALTCCPDKNYVQDKVCSPWSATVVATAIDNVLYTNNINQNVVGTGFVKYDVGPGPITVEALDSAGTVIDTQTLNPGTSIGFTYRRFDIIQVVLPATPAGTYQGEFCITTRYPLS

SEQ ID NO: 27 - protein, 122 aa, Bacillus toyonensis
MSCECSGSALTCCPDKNYVQDKVCSPWSATVVVTAIDNVLYTNNINQNVIGTGFVKYDVGPGPITVEALDSAGATIDTQTLNPGTSIAFTYRRFDSIQVVLPATPAGTYQGEFCITTRYPLS

SEQ ID NO: 28 - protein, 122 aa, Bacillus mycoides
MSCECSGSALTCCPDKNYVQDKVCSPWSATVVATAIDNVLYTNNINQNVIGTGFVKYDVGPGPITVEALDSAGATIDTQTLNPGTSIAFTYRRFDSIQVVLPATPAGTYQGEFCITTRYSLS

SEQ ID NO: 29 - protein, 117 aa, Bacillus cytotoxicus
MENYSANLSCCANSQKNIVQDKVCINWTASAAPAIIYADNIAQDIYASGYLKVDTGTGPVTLVFYSGGIGGTAVETITVATGSGASFTVRRFDTVTILGTASAETGEFCMTIRYTLI

SEQ ID NO: 30 - protein, 113 aa, Bacillus wiedmannii
LTCCPDKNYVQDKVCSPWSATVVATAIDNVLYTNNINQNVVGTGFVKYDVGPGPITVEVLDSAGTVLDTQTLNPGTSIGFTYRRFDIIQVVLPATPAGTYQGEFCITTRYPLS

SEQ ID NO: 31 - protein, 114 aa, Bacillus manliponensis
MGSNYSGLSCCSNKTIVQDKVCLDWSIDGTGAVVVYTNNVTQEIVASGYVKYDVGVGDITVEFLVGTTPVETLTISPGSSAAFTVRRFTDIEITTAGVGEHQGEFCITLRYPIS

SEQ ID NO: 32 - protein, 112 aa, Bacillus cereus
MGNSSLSCCSNNTLVQDQVCIDWSATGAVTETVYTNNITQDLYASGYVKYDVGTAPITVNFLVGATVVNTITVQPQSSGSFTVRYFTTIQIVTTGTGVSQGQLCLTVRYPIS

SEQ ID NO: 33 - protein, 110 aa, Bacillus thuringiensis
NSNLSCCSSKTLVQDQVCIDWAATGAVTETVYTNNITQDLYASGYVKYDVGTAPITVNFLVGATVVNTITVQPQSSGSFTVRYFTTIQIVTTGTGVSQGQLCLTVRYPIS

SEQ ID NO: 34 - protein, 111 aa, Bacillus mycoides
CNSNLSCCSNKTLVQDQVCIDWSATGAVTETVYTNNITQDLYASGYVKYDVGVNPVTVNFLVGATVVNTITIQPQSSGSFTVRYFTTIQIVTTGIGTSQGQLCLTVRYPIS

SEQ ID NO: 35 - protein, 114 aa, Bacillus anthracis
MGMRSSSLSCCSNKTLVQDQVCTDWSITGAGTQIVYTNNIAQEVYGSGYVKYDVGTAPITVDFLVGATVVDTITVQPQSSGTFTVRYFTTVRITTTGTTVNQGEFCITVRYPIS

SEQ ID NO: 36 - protein, 114 aa, Bacillus toyonensis
MGMRSSSLSCCSNKTLVQDQVCTDWSITGAGTQIVYTNNITQEVYGSGYVKYDVGANPITVDFLVGATVVDTITVQPQSSGTFTIRYFTTIRITTTGTTVSQGEFCITVRYPIS

SEQ ID NO: 37 - protein, 114 aa, Bacillus mycoides
MGMRSSSLSCCSNKTLVQDQVCTDWSITGAGNQIVYTNNLTQEIYGSGYVKYDTGANPITVDFLVGATVVDTITVQPQSSGTFTVRHFTTVRITTTGTTVNQGEFCITVRYSIS

SEQ ID NO: 38 - protein, 150 aa, Bacillus cytotoxicus
MQKHKKIGCFAPLSIICEVPCPPPPPISNAEFVTNEFVGNFLITNDTFLPETNQLKQSDSTFELWKGDGLSSLSGSISVYNNRNSTTAITVQIVGETTNTLTALPGNTVSYTGSGLQSVSLINIPSDPSVYIEGRYCCQITYCKFKSDCM

SEQ ID NO: 39 - protein, 135 aa, Bacillus thuringiensis
KKNIGCYAPLSIICPPYDPPNPPEPPGCELITNEFAGNFLITKEIPPPSENPTLILWEGDGVVKISGTISVYNSTSSTAGITVQIIGKVTNTFTVYPGNTMSYTGQSLQSVAIIDIPNNPTRYMEGKYCCQFSFC

SEQ ID NO: 40 - protein, 135 aa, Bacillus mycoides
KKNIGCYAPLSIICPPYDPPKPPKPPSCELVTNEFAGNFFITKEIPPPSENSTLVLWEGDGLLKISGTISVYNSTSSTGGITVQIIGQVTNTFTVYPGNTMSYTGQSLQSVTIVNIPNNPIQYIEGKYCCQFSFC

SEQ ID NO: 41 - protein, 135 aa, Bacillus wiedmannii
KKNIGCYAPLSIICPPYDPPNPPNPSSCELVNNEFAGNFFITKEIPPPSESSTLILWEGDGVLKISGTISVYNSTSSTEAVTVQIIGQVTNTFTVYPGNTMSYTGQSLQSLAIIDIPNNPTRYMEGKYCCQFSFC

SEQ ID NO: 42 - protein, 135 aa, Bacillus pacificus
KKNIGCYAPLSIICPPYDPPTPPDPPSCELVNNEFAGNFFITKEIPPPSEGSTLILWEGDGVLKISGNISVYNSTSSTEAVTVQIIGQVTNTFTVYPGNTMSYTGQSLQSVAIINLPNNPIRYIEGKYCCQFSFC

SEQ ID NO: 43 - protein, 134 aa, Bacillus thuringiensis
KNIGCYAPLSIICPPYDPPDPPNPPSCELVTNEFAGNFFITKEIPPPSKSPTLTLWEGDGILKISGTISVYNSTSSTEAVTVQIIGQVTNAFTVYPGNTMSYTGQSLQSMTIINIPHNPTRYIEGKYCCQFSFC

SEQ ID NO: 44 - protein, 143 aa, Bacillus mycoides
KKNIGCYAPLSIICPPYPPPNPPEPPGCELVMNEFAGNFFITNEIPLPPKENPNPFRTLPLWEGDGVLKISGTISIYNSTSSTGEVTVQIVGQVTSTFIFYPGNTMSYTGKSLQSVTIINIPNTPTQYIEGKYCCQFSFCLNR

SEQ ID NO: 45 - protein, 136 aa, Bacillus anthracis
KNIGCFAPLSIICPDPCPSTPPPNPNCERVTNEFAGNFLITKNTMPSAKGASQSMILWQSDGILLISGTVSVYNSTSSTEAITIEIVGAVTNIFTMFPGNTISYTGKDLQSVSIANIQHNPSLYLEGKYCCQFTCC

SEQ ID NO: 46 - protein, 136 aa, Bacillus wiedmannii
KNIGCFAPLSIICPDPCPPPPPPNPSCERVTNEFAGNFLITNNTISSVKDTSQSMILWQSDGILLISGTVSVYNSTSSTEAITIEIVGAVTNIFTVFPGNTISYTGKNLQSVSITNIQNNPSLYLEGKYCCQFTCC

SEQ ID NO: 47 - protein, 145 aa, Bacillus manliponensis
LSNRKRIGCFAPLGMNCYPPSPPLFPPPKPIETELITNEFCGNFLVTDQPLPSRETLELWQRDETSLITGTVSIYNSSNSTHPVSIQIIDKIAHQFIVFPGNTTSFTGNALQSISIIDIPNDSPLYIEGKYCCQFTYCRKKMNCM

SEQ ID NO: 48 - protein, 140 aa, Bacillus mycoides
LNKKKQIGCYAPLELSCHPIYPPCPPQPNPGCELVSNEFSGNFLVRDGQVQTLQLWKSDGITSISGTISVYNSSNSTDPATIVISGISTTTLIVLPGNTSSFTGTDLQSVEMIDIPNTSLSYLEGKYCCQFTYCHSKSNA

SEQ ID NO: 49 - protein, 113 aa, Bacillus cereus
MAQIGNCCTEQLCCVNDAVCCTIILDDTGGTALPIWDDATTFVINGTIMVENNGTVGVGPTAALTVNGTAVGGFVVAPGERSITMNDINSIAIVGAGTGTSSVKISFSINYKF

SEQ ID NO: 50 - protein, 112 aa, Bacillus sp. ms-22
MLGGCSNEQAGFVNDAVCFTLDLAAAADAVVVWTDGTPYVINGTIMGENNGIVAVAPTVTLEINGTAVPDFTVPPGESRSITLSDLNSIGIVATGGTSTGNIVVSFSLNYQY

SEQ ID NO: 51 - protein, 114 aa, Bacillus sp. LLTC93
MAILGGCSNEQTGFVNDAVCFTVDLAAAADAVVVWTDGTPFVINGTIMVENNGIVAVAPTVTLEVNGTAVPDFTVPPGESRSITLNDINSIGIVGTGGTSTGNIVVSFSLNYQY

SEQ ID NO: 52 - protein, 115 aa, Bacillus pumilus
MAILGGCSNEQTGFVNDAVCFTVDLGATAPDAVVVWTDGTPFVINGTIMVENNGIAAVAPTVSLQVNDAAVPGFTVPPGESRSITLNDINSIGIIGTGGTSTGNIVVSFSLNYQY

SEQ ID NO: 53 - protein, 115 aa, Bacillus atrophaeus
MAMLGGCSNEQMGFVNDAVCFTIDLGDTGAVATPVWEDGTSFVINGTIMVENNGIVGVGPTASLTVNGTAVAGFTVAPGESRSITLNDINSIGIVGTGGTGTANVTVSFSLNYQY

SEQ ID NO: 54 - protein, 116 aa, Peribacillus psychrosaccharolyticus
MAQIGSCNGEQLVGVNDSVCLTLELDDAVAPTVVWTDVTPYVINGTIMIENNGIVGVGQDAALSVNGTAIADFVVVPGEARSITLDNINSISLLGSGGEAGASSSVNVAFSLNYKF

SEQ ID NO: 55 - protein, 115 aa, Amphibacillus marinus
MAQLGSCCGDELVGVNDAVCFTVDLEATGEGADLILWEDETSFIINGSIMVENNGIIGASPTAALTVNGAAIPGFTVAPGEARSITLNDIETIGLTGTGTGTGSVKVSFSINYKY

SEQ ID NO: 56 - protein, 115 aa, Bacillus cereus
MALLGGCSDMSVSSVNDAVCFTITLEDTTAGTPVEVWEDSTGFAINGTIMIENNGITETSPTASLLVNDTEVTGFTVAPGESRAITLDNLNSIGISGAGTGSSAVKVSFSLNYKF

SEQ ID NO: 57 - protein, 116 aa, Bacillus endophyticus
MAQLGGCSSDELGVVNDAVCVTIDLPVTTVGTPIPVWTDTSTFAINGTIVVENNGTVGISPTASLEVNGTAVTDFTVGPGEAQSITLNNIESIAIAGTGGTGTAIVKVAFSLNYKF

SEQ ID NO: 58 - protein, 114 aa, Peribacillus loiseleuriae
MAQIGNCCSEQLVGVNDALCFTIDLADTATDTIDLWNNNTTFVINGTIMVENNGINGVTTTATLMVNGTAVTGFTVGPGEAMSVTMNNINSIGLVGAGTGTASVKVSFSLNYKF

SEQ ID NO: 59 - protein, 118 aa, Bacillus cereus
MAQIGNCCNDQLVGVNDALCFTIVLDDTNGTAIDLWNDATTFVINGTIMVENNGTIGVAPTASLTVNGTPVGGFIVGPGEARSITMNDINSIGIVGAGGTPVGSTANVKVSFSLNYKF

SEQ ID NO: 60 - protein, 118 aa, Peribacillus loiseleuriae
MAQIGNCCNDSLVGVNDALCFTIDIADTDGTPLVLWNDATTFVINGTIMVENNGTIGVSPTAALTVNGTPIAGFIVGPGEAKSVTLNDINSIGIIGTGGTPAGSTASVKVSFSLNYKF

SEQ ID NO: 61 - protein, 118 aa, Bacillus megaterium
MAQLGSCCSDSLGVVNDAVCFTVDLADTGGTEIVLWNDSTTFIINGTVMVENNGVTGVGPTASLTVNGVAVAGFVVEPGSARAITVNDINTIGIIGTGGAPLGSTSNVKISFSLNYKF

SEQ ID NO: 62 - protein, 118 aa, Bacillus aryabhattai
MADLGSCCSDNLGVVNDAVCFTISLGDTDGSPISIWSNGTTFVVNGTIMVENNGLANVGSTASLFVNGSAIEGFVVAPGEARAITINDINIIGIVGAGGTPVGSTSKVKISFSINYKF

SEQ ID NO: 63 - protein, 114 aa, Lysinibacillus sinduriensis
MAQLGGCNGEQLVGVNDAICFTVLLDDTGATPLPIWSDATTYIINGTIMVENNGVVGASPTAALTVNGTAVDGFVVAPGEARSITMNDINSIALTGTGAGTANVKVSFSLNYKF

SEQ ID NO: 64 - protein, 115 aa, Bacillus megaterium
MAQLGGCSSEGDFGSVNDAVCTTVELSDTGVTPITIWADTTSYIINGTILIENNGIIGASPTASLLVNGTPVADFTVAAGEARSLTVDNINLIQLAGVGAGTARVKVSFSLNYQF

SEQ ID NO: 65 - protein, 114 aa, Bacillus sp. YR335
MSKLGNCCQDQLVGVNDALCFTINLTNTGGSPIPIWDDATSFNINGTILIENKGVIGASPTAALVVNGTPVAGFIVGAGEVRSITMSNINSIRITGAGTGTAPVMISFSINYKY

SEQ ID NO: 66 - protein, 113 aa, Vibrio vulnificus
MQYGNCCNGLFGIVSDAVCFTVNLSNTAGVALELWNDATPFIINGTIVVENNGIIGASPTAALSVNGTPVGGFIVTAGQVKSITMNNINSIGLIGSGTGTASVRVSFSLNYKF

SEQ ID NO: 67 - protein, 114 aa, Peribacillus asahii
MANLGGFSNNPLNVISDSVSCTIESGDTNGAATTIWQDDTPFVINGTIVVDNKGKVGASPKAALLVNGTAINGFVVAPGECRSITVNDLHSISIIVTGTGTAKINISFSLNYKF

SEQ ID NO: 68 - protein, 116 aa, Bacillus cereus
MAQLGNCSNSQQNTVNDTVCNNIVLEDTAGVPISIWDNNTNMIINGTILVQNNRIIGIGTTTALTVNDTTVCGFVVRPGESRSITMNDINSIGIVGVGAGVNSSNVKISFSINYKF

SEQ ID NO: 69 - protein, 114 aa, Bacillus cereus
MTQLGNYSSSQQSTVNDAICSDIVLEDTAGVPVPIWDDNTNMIINGTILVQNNGIIGVGATAALIVNGTPVGGFIVGPGESRSITINDINSIGIVGAGTNSSNIKVSFSINYKF

SEQ ID NO: 70 - protein, 114 aa, Bacillus cereus group
MAQLGNCSNFQQQAVNDAVCCNIVLSDTGGTPVPIWNDDTSRIINGTILVQNDGIIGVGATASLIVNGTAVGGFTVGPGESRSITMSDINSIGIVGAGTNSSNVKVSFSINYKF

SEQ ID NO: 71 - protein, 114 aa, Halobacillus
MAQLGSCISNSYCCVNDAVCCTISLEDTGEIPVTVWEDVTDFVINGTIMIENNGITDTSPTAALYVNGEAIGDFVLAPGECRSITLNDINSIGIVGAGTGSSNVKVSFSINYKF

SEQ ID NO: 72 - protein, 114 aa, Virgibacillus dokdonensis
MAQLGLCNTDSLCCINDAVCCTITLEDTATTPISVWENASTFIINGTIVIENNGLVVGSPTAELYVNGTAVSGFVVEPGECRSITLEGVNSIGIVGSGTGSSNVKISFSINYKF

SEQ ID NO: 73 - protein, 114 aa, Bacillus sp. BPN334
MAQIGNCCTEQLCCVNDAVCCTIILDDTDGTALPIWDDSTTFVINGTIMVENNGAVGVGSTAALTVNGTAVGGFVVAPGECRSITMNDINSIAIVGSGTGTSSVKISFSLNYKF

SEQ ID NO: 74 - protein, 115 aa, Bacillus cereus ATCC 10987
MLAQIGNCCTEQLCCVNDAVCCTIILDDTGGTALPIWDDATTFVINGTIMVENNGTVGVGPTAALTVNGTAVGGFVVAPGECRSITMNDINSIAIVGAGTGTSSVKISFSINYKF

SEQ ID NO: 75 - protein, 114 aa, Paenibacillus sp. GM1FR
MAQIGNCNNTVSPAVVNDAVCTTISLSDTGGTPTIIYENSSTFLINGTILIENNGVGVSPTAALLIDGVQIPGFVVAPGSSRSYTAPTINSISLIGAGLGTTSIKIAFSLNYRF

SEQ ID NO: 76 - protein, 115 aa, Bacillus aurantiacus
MAQLGSCGRQQQDCCVNDAVCFNIEIETGGEEQLVWTNNGAFTINGTIVIENDGIEEGPSASLVVNGVATGVVVAPGESEAVTLDNLNSISVAPEGGGEGLTSDVKVAFSINYRF

SEQ ID NO: 77 - protein, 113 aa, Rummeliibacillus pycnus
MAKLGNCHMEPCCVNDAVCSTVHVVNQSLVTVWTNESPFNINGTIVVENPLQDGNATQQTVRLIITGSSGTDVNIAPGQTVALTVRGLHIIQVQGLTTGTTDVKVSFSLNYKF

SEQ ID NO: 78 - protein, 109 aa, Anaerobacillus alkalidiazotrophicus
MAQLGKCFPTTLCCVNDAVCCDISVTVGTGPVDLYTNSTNFPVNGTFMVDNSNSSTGNVTLTDGTNAIDVAPGTCQAITYSDINNGESITWQTTAAADFKVSFSINYKF

SEQ ID NO: 79 - protein, 111 aa, Lysinibacillus meyeri
MAKLGSCCGSKTSVHDSVCQDVTLTDETPVIVWENSTPFSINGTILVENASSSTADVTLTVTSNPATPAIAAIAPGNSVSVTANNISDIQLAGEAAEVANVKISYSLNYNF

SEQ ID NO: 80 - protein, 111 aa, Metasolibacillus fluoroglycofenilyticus
MAKLGSCCGSKTSVHDSVCQDLTLTDETAVTVWENSTPFAINGTILVENASSSTGDVTLTVTSSPATPAIPPITPGNSISITANNIADIQLAGTAASVANVKISYSLNYNF

SEQ ID NO: 81 - protein, 122 aa, Bacillus thuringiensis
MSCECSGSALTCCPDKNYVQDKVCSPWSATVVATAIDNVLYTNNINQNVVGTGFVKYDVGPGPITVEALDSAGGTIDTQTLNPGTSIAFTYRRFDSIQVVLPATPAGTYQGEFCITTRYPLS

SEQ ID NO: 82 - protein, 141 aa, Bacillus thuringiensis
MKNKKNIGCFAPLSIICPDPCPPPPPPNPNCERVTNEFAGNFLITNNTIPSAKDASQSMILWQSDGILLISGTVSVYNSTSSTETITIQIVGTVTNIFTVFPGNTISYTGKDLHSVSIINITSNPSLYLEGKYCCEFTCCL

SEQ ID NO: 83 - DNA, 981 bp, synthetic construct
  source 1..981: mol_type = other DNA
atgaaaaaatatgtttgtaccgtttgcggatatgagtatgatccagccgagggcgacccagacaacggtgtaaaaccaggcacttcttttgacgacctgcccgccgactgggtatgtccggtctgtggagcacctaaaagtgagttcgaggccgcgtaaatggtcaccaaaaaggcctctcacaaagacgcaggctatcaggagtctccaaacggcgcaaaacgttgcgggacgtgccggcagtttcgaccgccgtcttcctgcatcacagtggagagtccgatttccgaaaacggttggtgtagactgtatgcagggaaagcatgaatgccccctgaacagactccggtgtgggaatgcgagtcagaagactgtctggggtggatgcgtaagaatttctcgtttgaagaagaacctaaatgtccattgtgtaaatcatcaatgaagagcggtgagcgcctcctcccaaaactcggttagatgtgcgttctctgtcgcgataagggaattatacgtaaagaaatctacccaggggttgccctgaccgaaggttgcaactgcgaataaatggttgtgatctgtcccaattgtcagagctctgcagagattattcacgtactgaccgcacaaagtaatcagaacgtgatttatacatgtcaggtatgtcactttgtaattcgcaatatcgaataaatgaaactttgtttctattgttttaaaattcaatctaagaagacgaagattatcggtgatgccgaggggcggatgcaaatttgtgaaacatgtcataaggagctgttctgaatgttagtctgtcctaaatgcgggagtgcgcaggcccgtagtggagggtaccaggaatgctgcaaaattcgcttatgaatgcagctctgtagttgttgtggttatcagaacaaagatgtcaaaaacctgaatctgcgcgaatgggactgccctagttgtcgcacccatcatgactag

SEQ ID NO: 84 - DNA, 18 bp, synthetic construct
  source 1..18: mol_type = other DNA
gtgaccatcgtcttttac

SEQ ID NO: 85 - DNA, 18 bp, synthetic construct
  source 1..18: mol_type = other DNA
acccgtacctgtatccac

SEQ ID NO: 86 - DNA, 18 bp, synthetic construct
  source 1..18: mol_type = other DNA
gcagcggagactggtgag

SEQ ID NO: 87 - DNA, 18 bp, synthetic construct
  source 1..18: mol_type = other DNA
gcccagaatagtgacggt

SEQ ID NO: 88 - DNA, 41 bp, synthetic construct
  source 1..41: mol_type = other DNA
gatacaggtacgggtggaaaatatgtttgtaccgtttgtac

SEQ ID NO: 89 - DNA, 44 bp, synthetic construct
  source 1..44: mol_type = other DNA
gtcactattctgggcggaggaaaatatgtttgtaccgtttgtac

SEQ ID NO: 90 - DNA, 31 bp, synthetic construct
  source 1..31: mol_type = other DNA
aaagacgatggtcacccgcgcggcctcgaac

SEQ ID NO: 91 - DNA, 31 bp, synthetic construct
  source 1..31: mol_type = other DNA
accagtctccgctgcgcccgcggcctcgaac

SEQ ID NO: 92 - DNA, 30 bp, synthetic construct
  source 1..30: mol_type = other DNA
gatacaggtacgggtgtcaccaaaaaggcc

SEQ ID NO: 93 - DNA, 33 bp, synthetic construct
  source 1..33: mol_type = other DNA
gtcactattctgggcggagtcaccaaaaaggcc

SEQ ID NO: 94 - DNA, 34 bp, synthetic construct
  source 1..34: mol_type = other DNA
aaagacgatggtcacccgtgctttccctgcatac

SEQ ID NO: 95 - DNA, 34 bp, synthetic construct
  source 1..34: mol_type = other DNA
accagtctccgctgcgcctgctttccctgcatac

SEQ ID NO: 96 - DNA, 30 bp, synthetic construct
  source 1..30: mol_type = other DNA
gatacaggtacgggtgaacagactccggtg

SEQ ID NO: 97 - DNA, 33 bp, synthetic construct
  source 1..33: mol_type = other DNA
gtcactattctgggcggagaacagactccggtg

SEQ ID NO: 98 - DNA, 32 bp, synthetic construct
  source 1..32: mol_type = other DNA
aaagacgatggtcacccggaggaggcgctcac

SEQ ID NO: 99 - DNA, 32 bp, synthetic construct
  source 1..32: mol_type = other DNA
accagtctccgctgcgccgaggaggcgctcac

SEQ ID NO: 100 - DNA, 32 bp, synthetic construct
  source 1..32: mol_type = other DNA
gatacaggtacgggtggctgcgttctctgtcg

SEQ ID NO: 101 - DNA, 35 bp, synthetic construct
  source 1..35: mol_type = other DNA
gtcactattctgggcggaggctgcgttctctgtcg

SEQ ID NO: 102 - DNA, 36 bp, synthetic construct
  source 1..36: mol_type = other DNA
aaagacgatggtcacccggccttcgcagttgcaacc

SEQ ID NO: 103 - DNA, 36 bp, synthetic construct
  source 1..36: mol_type = other DNA
accagtctccgctgcgccgccttcgcagttgcaacc

SEQ ID NO: 104 - DNA, 35 bp, synthetic construct
  source 1..35: mol_type = other DNA
gatacaggtacgggtggcgttgtgatctgtcccaa

SEQ ID NO: 105 - DNA, 38 bp, synthetic construct
  source 1..38: mol_type = other DNA
gtcactattctgggcggaggcgttgtgatctgtcccaa

SEQ ID NO: 106 - DNA, 46 bp, synthetic construct
  source 1..46: mol_type = other DNA
aaagacgatggtcacccggcctgattcgatattgcgaattacaaag

SEQ ID NO: 107 - DNA, 46 bp, synthetic construct
  source 1..46: mol_type = other DNA
accagtctccgctgcgccgcctgattcgatattgcgaattacaaag

SEQ ID NO: 108 - DNA, 49 bp, synthetic construct
  source 1..49: mol_type = other DNA
gatacaggtacgggttctgcaaaactttgtttctattgttttaaaattc

SEQ ID NO: 109 - DNA, 52 bp, synthetic construct
  source 1..52: mol_type = other DNA
gtcactattctgggcggatctgcaaaactttgtttctattgttttaaaattc

SEQ ID NO: 110 - DNA, 36 bp, synthetic construct
  source 1..36: mol_type = other DNA
aaagacgatggtcacccggcccagctccttatgaca

SEQ ID NO: 111 - DNA, 36 bp, synthetic construct
  source 1..36: mol_type = other DNA
accagtctccgctgcgccgcccagctccttatgaca

SEQ ID NO: 112 - DNA, 37 bp, synthetic construct
  source 1..37: mol_type = other DNA
gatacaggtacgggtgcattagtctgtcctaaatgcg

SEQ ID NO: 113 - DNA, 40 bp, synthetic construct
  source 1..40: mol_type = other DNA
gtcactattctgggcggagcattagtctgtcctaaatgcg

SEQ ID NO: 114 - DNA, 35 bp, synthetic construct
  source 1..35: mol_type = other DNA
aaagacgatggtcacccgtaagcgaattttgcagc

SEQ ID NO: 115 - DNA, 35 bp, synthetic construct
  source 1..35: mol_type = other DNA
accagtctccgctgcgcctaagcgaattttgcagc

SEQ ID NO: 116 - DNA, 38 bp, synthetic construct
  source 1..38: mol_type = other DNA
gatacaggtacgggtgcatctcagctctgtagttgttg

SEQ ID NO: 117 - DNA, 41 bp, synthetic construct
  source 1..41: mol_type = other DNA
gtcactattctgggcggagcatctcagctctgtagttgttg

SEQ ID NO: 118 - DNA, 32 bp, synthetic construct
  source 1..32: mol_type = other DNA
aaagacgatggtcacccggtcatgatgggtgc

SEQ ID NO: 119 - DNA, 32 bp, synthetic construct
  source 1..32: mol_type = other DNA
accagtctccgctgcgccgtcatgatgggtgc

SEQ ID NO: 120 - DNA, 24 bp, synthetic construct
  source 1..24: mol_type = other DNA
ggtgatgccggccacgatgcgtcc

SEQ ID NO: 121 - DNA, 24 bp, synthetic construct
  source 1..24: mol_type = other DNA
ggttatgctagttattgctcagcg

SEQ ID NO: 122 - DNA, 19 bp, synthetic construct
  source 1..19: mol_type = other DNA
taatacgactcactatagg

SEQ ID NO: 123 - DNA, 19 bp, synthetic construct
  source 1..19: mol_type = other DNA
gctagttattgctcagcgg

SEQ ID NO: 124 - protein, 5 aa, synthetic construct
TGTGP

SEQ ID NO: 125 - protein, 4 aa, synthetic construct
TGTG

SEQ ID NO: 126 - protein, 5 aa, synthetic construct
  VARIANT 5: note = R or G
TGTGX

SEQ ID NO: 127 - protein, 5 aa, synthetic construct
GTAAA

SEQ ID NO: 128 - protein, 5 aa, synthetic construct
GGGAA

SEQ ID NO: 129 - protein, 50 aa, synthetic construct
KYVCTVCGYEYDPAEGDPDNGVKPGTSFDDLPADWVCPVCGAPKSEFEAA

SEQ ID NO: 130 - protein, 54 aa, synthetic construct
VTKKASHKDAGYQESPNGAKRCGTCRQFRPPSSCITVESPISENGWCRLYAGKA

SEQ ID NO: 131 - protein, 43 aa, synthetic construct
EQTPVWECESEDCLGWMRKNFSFEEEPKCPLCKSSMKSGERLL

SEQ ID NO: 132 - protein, 27 aa, synthetic construct
CVLCRDKGIIRKEIYPGVALTEGCNCE

SEQ ID NO: 133 - protein, 40 aa, synthetic construct
VVICPNCQSSAEIIHVLTAQSNQNVIYTCQVCHFVIRNIE

SEQ ID NO: 134 - protein, 34 aa, synthetic construct
KLCFYCFKIQSKKTKIIGDAEGRMQICETCHKEL

SEQ ID NO: 135 - protein, 24 aa, synthetic construct
LVCPKCGSAQARSGGYQECCKIRL

SEQ ID NO: 136 - protein, 31 aa, synthetic construct
QLCSCCGYQNKDVKNLNLREWDCPSCRTHHD

SEQ ID NO: 137 - DNA, 339 bp, synthetic construct
  source 1..339: mol_type = other DNA
atgggtaactgcagcaccaatctgtcatgctgtgccaatggtcagaagaccattgtccaggataaagtctgcatcgactggaccgcagccgctactgcagcaatcatttacgctgataatatcagccaagacatctacgcttcaggctatctgaaagtggatgtgaccatcgtcttttactctggtggagtcacaggcaccgctgtggagaccattgtggtcgccacgggttcgtcggccagctttacggtgcgccgttttgataccgtcactattctgggcaccgcagcagcggagactggtgagttttgtatgaccatccgttacactttgagctaa

SEQ ID NO: 138 - protein, 112 aa, synthetic construct
MGNCSTNLSCCANGQKTIVQDKVCIDWTAAATAAIIYADNISQDIYASGYLKVDVTIVFYSGGVTGTAVETIVVATGSSASFTVRRFDTVTILGTAAAETGEFCMTIRYTLS

SEQ ID NO: 139 - DNA, 339 bp, synthetic construct
  source 1..339: mol_type = other DNA
atgggtaactgcagcaccaatctgtcatgctgtgccaatggtcagaagaccattgtccaggataaagtctgcatcgactggaccgcagccgctactgcagcaatcatttacgctgataatatcagccaagacatctacgcttcaggctatctgaaagtggatacaggtacgggtcccgtgaccatcgtcttttactctggtggagtcacaggcaccgctgtggagaccattgtggtcgccacgggttcgtcggccagctttacggtgcgccgttttgataccgtcactattctggagactggtgagttttgtatgaccatccgttacactttgagctaa

SEQ ID NO: 140 - protein, 112 aa, synthetic construct
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILETGEFCMTIRYTLS

SEQ ID NO: 141 - protein, 56 aa, synthetic construct
  VARIANT 56: note = G or R
TGTGGKYVCTVCGYEYDPAEGDPDNGVKPGTSFDDLPADWVCPVCGAPKSEFEAAX

SEQ ID NO: 142 - protein, 56 aa, synthetic construct
GGGKYVCTVCGYEYDPAEGDPDNGVKPGTSFDDLPADWVCPVCGAPKSEFEAAGAA

SEQ ID NO: 143 - protein, 59 aa, synthetic construct
  VARIANT 59: note = G or R
TGTGVTKKASHKDAGYQESPNGAKRCGTCRQFRPPSSCITVESPISENGWCRLYAGKAX

SEQ ID NO: 144 - protein, 59 aa, synthetic construct
GGVTKKASHKDAGYQESPNGAKRCGTCRQFRPPSSCITVESPISENGWCRLYAGKAGAA

SEQ ID NO: 145 - protein, 48 aa, synthetic construct
  VARIANT 48: note = G or R
TGTGEQTPVWECESEDCLGWMRKNFSFEEEPKCPLCKSSMKSGERLLX

SEQ ID NO: 146 - protein, 48 aa, synthetic construct
GGEQTPVWECESEDCLGWMRKNFSFEEEPKCPLCKSSMKSGERLLGAA

SEQ ID NO: 147 - protein, 34 aa, synthetic construct
  VARIANT 34: note = G or R
TGTGGCVLCRDKGIIRKEIYPGVALTEGCNCEGX

SEQ ID NO: 148 - protein, 34 aa, synthetic construct
GGGCVLCRDKGIIRKEIYPGVALTEGCNCEGGAA

SEQ ID NO: 149 - protein, 48 aa, synthetic construct
  VARIANT 48: note = G or R
TGTGGVVICPNCQSSAEIIHVLTAQSNQNVIYTCQVCHFVIRNIESGX

SEQ ID NO: 150 - protein, 48 aa, synthetic construct
GGGVVICPNCQSSAEIIHVLTAQSNQNVIYTCQVCHFVIRNIESGGAA

SEQ ID NO: 151 - protein, 42 aa, synthetic construct
  VARIANT 42: note = G or R
TGTGSAKLCFYCFKIQSKKTKIIGDAEGRMQICETCHKELGX

SEQ ID NO: 152 - protein, 42 aa, synthetic construct
GGSAKLCFYCFKIQSKKTKIIGDAEGRMQICETCHKELGGAA

SEQ ID NO: 153 - protein, 30 aa, synthetic construct
  VARIANT 30: note = G or R
TGTGALVCPKCGSAQARSGGYQECCKIRLX

SEQ ID NO: 154 - protein, 30 aa, synthetic construct
GGALVCPKCGSAQARSGGYQECCKIRLGAA

SEQ ID NO: 155 - protein, 38 aa, synthetic construct
  VARIANT 38: note = G or R
TGTGASQLCSCCGYQNKDVKNLNLREWDCPSCRTHHDX

SEQ ID NO: 156 - protein, 38 aa, synthetic construct
GGASQLCSCCGYQNKDVKNLNLREWDCPSCRTHHDGAA

SEQ ID NO: 157 - protein, 137 aa, Sycon ciliatum
MRSVGKNILVRSDNDDSNNDSNDGSAMNSGMGESDSSDGGDDSSDDSDDSNNEDDSDSEDSENGSNSDGSDSDDGSAVQAQQPAVLVPAVPETPRPPIFITQPIDSDRDAPQTNAPTEALMADPPTPAAPLDKDAPQ

SEQ ID NO: 158 - protein, 256 aa, synthetic construct
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGGGMRSVGKNILVRSDNDDSNNDSNDGSAMNSGMGESDSSDGGDDSSDDSDDSNNEDDSDSEDSENGSNSDGSDSDDGSAVQAQQPAVLVPAVPETPRPPIFITQPIDSDRDAPQTNAPTEALMADPPTPAAPLDKDAPQGGAAETGEFCMTIRYTLS

SEQ ID NO: 159 - protein, 19 aa, synthetic construct
NDSDTDQDGGGNDSDTDQD

SEQ ID NO: 160 - protein, 41 aa, synthetic construct
NDSDTDQDGGGNDSDTDQDGGGNDSDTDQDGGGNDSDTDQD

SEQ ID NO: 161 - protein, 19 aa, synthetic construct
NDADIDQDGSENDAAIEQD

SEQ ID NO: 162 - protein, 42 aa, synthetic construct
NDADIDQDGSENDAAIEQDGGEDNDADIDQDGSENDAAIEQD

SEQ ID NO: 163 - protein, 19 aa, synthetic construct
NDADIDQDGSENDADIDQD

SEQ ID NO: 164 - protein, 42 aa, synthetic construct
NDADIDQDGSENDADIDQDGGEDNDADIDQDGSENDADIDQD

SEQ ID NO: 165 - protein, 139 aa, synthetic construct
GMRSVGKNILVRSDNDDSNNDSNDGSAMNSGMGESDSSDGGDDSSDDSDDSNNEDDSDSEDSENGSNSDGSDSDDGSAVQAQQPAVLVPAVPETPRPPIFITQPIDSDRDAPQTNAPTEALMADPPTPAAPLDKDAPQG

SEQ ID NO: 166 - protein, 28 aa, synthetic construct
GGGSNDSDTDQDGGGNDSDTDQDGSGAA

SEQ ID NO: 167 - protein, 51 aa, synthetic construct
GGSGDNDSDTDQDGGGNDSDTDQDGGGNDSDTDQDGGGNDSDTDQDGSGAA

SEQ ID NO: 168 - protein, 27 aa, synthetic construct
GGGSNDADIDQDGSENDAAIEQDGGAA

SEQ ID NO: 169 - protein, 52 aa, synthetic construct
GGSGDNDADIDQDGSENDAAIEQDGGEDNDADIDQDGSENDAAIEQDGSGAA

SEQ ID NO: 170 - protein, 27 aa, synthetic construct
GGGSNDADIDQDGSENDADIDQDGGAA

SEQ ID NO: 171 - protein, 52 aa, synthetic construct
GGSGDNDADIDQDGSENDADIDQDGGEDNDADIDQDGSENDADIDQDGSGAA

SEQ ID NO: 172 - protein, 139 aa, synthetic construct
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGGGSNDSDTDQDGGGNDSDTDQDGGAAETGEFCMTIRYTLS

SEQ ID NO: 173 - protein, 163 aa, synthetic construct
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGGSGDNDSDTDQDGGGNDSDTDQDGGGNDSDTDQDGGGNDSDTDQDGSGAAETGEFCMTIRYTLS

SEQ ID NO: 174 - protein, 139 aa, synthetic construct
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGGGSNDADIDQDGSENDAAIEQDGGAAETGEFCMTIRYTLS

SEQ ID NO: 175 - protein, 163 aa, synthetic construct
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGGGSNDADIDQDGSENDAAIEQDGGEDNDADIDQDGSENDAAIEQDGSGAAETGEFCMTIRYTLS

SEQ ID NO: 176 - protein, 140 aa, synthetic construct
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGGSGDNDADIDQDGSENDADIDQDGGAAETGEFCMTIRYTLS

SEQ ID NO: 177 - protein, 164 aa, synthetic construct
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGGSGDNDADIDQDGSENDADIDQDGGEDNDADIDQDGSENDADIDQDGSGAAETGEFCMTIRYTLS

SEQ ID NO: 178 - protein, 19 aa, synthetic construct
NXAXIXQXGSENXAXIXQD

SEQ ID NO: 179 - protein, 19 aa, synthetic construct
NXSXTXQXGGGNXSXTXQD

SEQ ID NO: 180 - protein, 4 aa, synthetic construct
GGGS

SEQ ID NO: 181 - protein, 4 aa, synthetic construct
GGAA

SEQ ID NO: 182 - protein, 5 aa, synthetic construct
GGSGD

SEQ ID NO: 183 - protein, 5 aa, synthetic construct
GSGAA

SEQ ID NO: 184 - protein, 4 aa, synthetic construct
GGED

SEQ ID NO: 185 - protein, 176 aa, synthetic construct
MACECSSTVLTCCSDNSSNFVQDKVCNPWSSAEASTFTVYANNVNQNIVGTGYLTYDVGPGVSPAGKYVCTVCGYEYDPAEGDPDNGVKPGTSFDDLPADWVCPVCGAPKSEFEAAGITVTVLDSGGGTIQTFLVNEGTSISFTFRRFNIIQITTPATPIGTYQGEFCITTRYLMA

SEQ ID NO: 186 - protein, 11 aa, synthetic construct
VGPGVSPANQI

SEQ ID NO: 187 - protein, 8 aa, synthetic construct
TPATPIGT

SEQ ID NO: 188 - DNA, 24 bp, synthetic construct
  source 1..24: mol_type = other DNA
attacggttacggttctggactcc

SEQ ID NO: 189 - DNA, 14 bp, synthetic construct
  source 1..14: mol_type = other DNA
ggccgggctcacac

SEQ ID NO: 190 - DNA, 41 bp, synthetic construct
  source 1..41: mol_type = other DNA
ggtgtgagcccggccggaaaatatgtttgtaccgtttgcgg

SEQ ID NO: 191 - DNA, 31 bp, synthetic construct
  source 1..31: mol_type = other DNA
aaccgtaaccgtaattcccgcggcctcgaac

SEQ ID NO: 192 - protein, 31 aa, Oryctolagus cuniculus
MDPNCSCAAAGDSCTCANSCTCKACKCTSCK

SEQ ID NO: 193 - protein, 40 aa, Saccharomyces cerevisiae
QNEGHECQCQCGSCKNNEQCQKSCSCPTGCNSDDKCPCGN

SEQ ID NO: 194 - protein, 163 aa, synthetic construct
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGGGSGQNEGHECQCQCGSCKNNEQCQKSCSCPTGCNSDDKCPCGNGAGGAAETGEFCMTIRYTLS

SEQ ID NO: 195 - protein, 154 aa, synthetic construct
MGNCSTNLSCCANGQKTIVQDKVCIDWTAAATAAIIYADNISQDIYASGYLKVDTGTGPVTIVFYSGGVTGTAVETIVVATGSSASFTVRRFDTVTILGGGSGMDPNCSCAAAGDSCTCANSCTCKACKCTSCKGAGGAAETGEFCMTIRYTLS

SEQ ID NO: 196 - protein, 179 aa, synthetic construct
MGNCSTNLSCCANGQKTIVQDKVCIDWTAAATAAIIYADNISQDIYASGYLKVDTGSYPYDVPDYAGSGPVTIVFYSGGVTGTAVETIVVATGSSASFTVRRFDTVTILGGGKYVCTVCGYEYDPAEGDPDNGVKPGTSFDDLPADWVCPVCGAPKSEFEAAGAAETGEFCMTIRYTLS

SEQ ID NO: 197 - protein, 168 aa, synthetic construct
MGNCSTNLSCCANGQKTIVQDKVCIDWTAAATAAIIYADNISQDIYASGYLKVDTGTGGKYVCTVCGYEYDPAEGDPDNGVKPGTSFDDLPADWVCPVCGAPKSEFEAARVTIVFYSGGVTGTAVETIVVATGSSASFTVRRFDTVTILGTAAAETGEFCMTIRYTLS

SEQ ID NO: 198 - protein, 168 aa, synthetic construct
MGNCSTNLSCCANGQKTIVQDKVCIDWTAAATAAIIYADNISQDIYASGYLKVDTGTGGKYVCTVCGYEYDPAEGDPDNGVKPGTSFDDLPADWVCPVCGAPKSEFEAAGVTIVFYSGGVTGTAVETIVVATGSSASFTVRRFDTVTILGTAAAETGEFCMTIRYTLS

(19) United States
(12) Patent Application Publication
(10) Pub. No.: US 2026/0139013 A1
(43) Pub. Date: May 21, 2026

(54) Metal-Binding Bacterial Protein Fibers

(71) Applicants: VIB VZW, Gent (BE); Vrije Universiteit Brussel, Brussel (BE)

(72) Inventors: Han Remaut, Roosbeek (BE); Mike Sleutel, Huizingen (BE); Inge Van Molle, Brussel (BE); Sylvia Sherdrack, Brussel (BE)

(21) Appl. No.: 19/120,687

(22) PCT Filed: Oct. 11, 2023
(86) PCT No.: PCT/EP2023/078128

§ 371 Date: Apr. 11, 2025

(30) Foreign Application Priority Data
Oct. 12, 2022 (EP) 22201184.3

(51) Int. Cl. C07K 14/32; C07K 14/33; G16B 15/30

(52) U.S. Cl. CPC C07K 14/32; C07K 14/33; G16B 15/30; C07K 2319/735

(57) ABSTRACT

The present invention relates to the field of bacterial protein engineering and protein fibers applicable as metal-binding bionanomaterials. More specifically, the present invention relates to engineered bacterial endospore appendage (Ena) proteins modified to contain metal-binding polypeptide (MBP) inserts providing for stable, flexible and robust protein assemblies with metal-binding activity. In particular, the invention relates to methods for designing Ena-fusion proteins capable of self-assembling into fibers, and for recombinant production of said self-assembling Ena-MBP fusion protein subunits, assemblies and fibers, ensuring a sustainable source of biological material for use in metal mineralization, metal sequestration, and metal-removal applications such as waste water treatment, water softening, or bioremediation.

### CROSS REFERENCE TO RELATED APPLICATIONS

This application is a national phase entry under 35 U.S.C. § 371 of International Patent Application PCT/EP2023/078128, filed Oct. 11, 2023, designating the United States of America and published in English as International Patent Publication WO2024/079161 on Apr. 18, 2024, which claims the benefit under Article 8 of the Patent Cooperation Treaty to European Patent Application Serial No. 22201184.3, filed Oct. 12, 2022, the entireties of which are hereby incorporated by reference.

### INCORPORATION BY REFERENCE

The ST.26 XML Sequence listing named “10488-10938-US-Sequence Listing”, created on Oct. 10, 2023, and having a size of 192,512 bytes, is hereby incorporated herein by this reference in its entirety.

### FIELD OF THE INVENTION

The present invention relates to the field of bacterial protein engineering and protein fibers applicable as metal-binding bionanomaterials. More specifically, the present invention relates to engineered bacterial endospore appendage (Ena) proteins modified to contain metal-binding polypeptide (MBP) inserts providing for stable, flexible and robust protein assemblies with metal-binding activity. In particular, the invention relates to methods for designing Ena-fusion proteins capable of self-assembling into fibers, and for recombinant production of said self-assembling Ena-MBP fusion protein subunits, assemblies and fibers, ensuring a sustainable source of biological material for use in metal mineralization, metal sequestration, and metal-removal applications such as waste water treatment, water softening, or bioremediation.

### BACKGROUND

Many biological systems contain fiber forming proteins, consisting of linear polymers or bundles of covalent or non-covalently associated polypeptide units. Various biomaterials, such as collagen, silk, and elastin, have been receiving human interest due to their specialised functions and characteristics. These fiber forming protein polymers have qualities such as a diversity in tensile strength, elasticity, and stability, that make them optimal for use in daily life. Challenges of applying such biomaterial, for instance with regards to difficulty in industrial production or for developing new applications, are tackled for instance through applying synthetic biology via redesigning or engineering of those polymeric or protein-based materials.

Nanowires, including metallic nanowires, are currently being developed for possible applications in the field of optics, genetics, and electronics. One of the current hurdles is to establish scalable production platforms for sustainable and biodegradable nanowires. Due to self-assembly mechanisms into highly fiber networks, bacterial pili and amyloids are interesting to engineer into nanowires, though also those currently prove challenging to utilize in industrial applications (Shapiro et al., 2022).

Endospore appendages were first documented 58 years ago, revealing that appendage formation depends on specific bacterial strains (Walker et al., 2007), such as for instance on Clostridium and Bacillus species including B. thuringiensis, as protrusions in different forms such as pili, ribbon-like, tails or feathers, brushes, swords, or tubules (Hachisuka et al., 1984; Rode and Slepecky, 1971; Ankolekar and Labbé, 2010). The structure and identity of any of these appendages was largely unknown, and only very recently cryogenic electron microscopy (Cryo-EM) was applied for resolving Endospore appendages from pathogenic Bacillus by Pradhan et al. (2021), reporting a new pilus superfamily with eccentric properties adjusted to the harsh conditions encountered by the spores of these bacteria. The Endospore appendage (ENA) ultrastructure on B. cereus spores thus opened a novel, distinct, highly stable, and flexible type of pili, assembled by lateral β-augmentation and longitudinal disulfide crosslinking of identical subunits, making them highly resistant to denaturation with high temperatures, desiccation, or chemical damage. Moreover, the recombinant production of such self-assembling ENA proteins into bacterially produced fibers provide for a manufacturable source of biomaterials (Remaut et al., WO2022/029325A2). Given the high structural sturdiness, flexibility and biodegradability of these endospore appendages, they might thus serve many applications in nanotechnology as biofibers or nano biodevices (Zegeye et al., 2021), though, engineerability of the ENA's would need to be further investigated, as to determine whether these could serve in fulfilling the needs in finding more suitable biodegradable nanowire alternatives.

Moreover, beyond their application as nanowires, engineering of bacterial protein fibers may also be desired for use in other biosustainable applications which currently suffer from a number of other issues. For instance, nanoparticles are applied in environmental clean-up and provide for a low-cost and efficient solution for limiting the spread of harmful contaminants in the environment (Kumar et al., 2022). Though the risk of nanoparticles' interactions with environmental constituents, which is still unknown has made the use of nanoparticles a contentious issue. Furthermore, biomineralization, which is the precipitation of a wide range of minerals by living organisms, is considered to have a higher long-term sustainability in comparison to more traditional techniques, and has been introduced to remove heavy metals or convert them from a soluble to an insoluble form from contaminated sites, converting them from a bioavailable to a non-bioavailable form, and is often proposed as the most suitable mechanism for carbonate precipitation, but requires improvements (Zhu et al., 2016; Rajasekar et al., 2021). Calcium is one of the hardness causing minerals most commonly released from industries that produce sodium carbonate. High concentrations of calcium in (waste) water treatment show higher scale deposition in pipelines and membranes. The scaling occurs due to the chemical reactions that lead to the formation of multiple calcium products. Since calcium is a vital element in calcium carbonate formation, soluble calcium can be converted into insoluble calcium carbonate and filtered out using Microbially Induced Carbonate Precipitation (MICP) or sedimentation. So, also in the applications of biomineralization and bioremediation many approaches are still challenged with several hurdles limiting commercial viability, though the utilization of synthetic biology or genetically modified microbial species combined with structure-based engineering can provide a sustainable alternative. So, there is an interest and need for novel engineering approaches to provide improved bacterial protein assemblies and fibers suitable as nanowires, or biological solutions for the environment, basically by investigating routes to design, generate, develop, and manufacture such biomaterials in a sustainable way.

### SUMMARY OF THE INVENTION

In this study, the engineerability of Ena protein fibers was investigated by modifying the recombinantly produced Ena1B fiber by grafting relatively small metal binding proteins in one of two loops (DE-loop or HI-loop) of the Ena1B and Ena1A protein subunits. Several metal binding polypeptides (MBPs) were first identified as candidate inserts, based on the criteria that those MBPs are required to possess a metal binding site, are relatively small as to avoid steric hindrance during self-assembly and/or fiber formation (so preferably with a molecular weight below 25 kDa, more preferably below 6 kDa or lower), and have an N- and C-terminus which are in each other's proximity when the polypeptide is in its functional or folded state. A number of non-limiting examples of MBPs include rubredoxin [2Fe-2S], high potential iron-sulfur proteins (HiPIPs) [4Fe-4S], hypothetical proteins WP_098221117.1, FC702_01375, WP_197262982.1, WP_000861196, WP_086405534.1, WP_142338290.1, diactinin, and curlin repeat or curlin-like repeat peptides engineered for calcium-binding, and metallothionein proteins, as discussed further herein. For the insertion into the Ena protein subunit, possible surface exposed loop openings for insertion were explored, resulting in the DE- or HI-loop interruption for insertion, as referred to in the examples, so engineered in that a number of residues were substituted, deleted, or added (linkers), and providing for cleavage sites wherein the fusion with an MBP is envisaged for forming Ena-MBP functional fusion protein assemblies. Moreover, the predicted folding capacity of the inserts and the envisaged engineered fusions was evaluated using AlphaFold2 predictions (Jumper et al., 2021). So the present invention provides for the first time Ena protein assemblies, such as Ena multimers and fibers, engineered to comprise folded MBPs presented on the Ena surface, and functional in that metal-binding is obtained. Such Ena-MBP protein materials are thus useful in several processes requiring metal-binding activity, especially where a sustainable, biodegradable and scalable nanomaterial is desired. Such applications are described also herein, including but not limited to, mineralisation and sequestration of metals, using a sedimentation or ultrafiltration method for Ena-MBPs when provided in metal-rich environments; or metal removal or exchange, such as for heavy metal removal aimed for during bioremediation, removal of pollutant metals in waste water, or in water softening of drinking water.

So a first aspect of the invention relates to the self-assembling Ena fusion protein comprising a metal-binding polypeptide (MBP) inserted into an Ena protein, which is defined as a protein family of proteins comprising a DUF3992 domain, wherein the protein has a three-dimensional predicted fold matching the Ena1B structure with a fold similarity Z-score of 6.5 or more wherein Ena1B corresponds to SEQ ID NO: 8, or a MBP inserted into an engineered Ena protein, as defined below, and wherein the insertion of the MBP sequence is made by fusing with the Ena protein sequence at an exposed loop of the Ena protein structure, wherein the link between the MBP sequence and the Ena sequence is made directly, or via a linker sequence flanking the N- and/or C-terminus of the MBP. So in a specific embodiment, the Ena fusion protein or Ena-MBP fusion, comprises an Ena protein selected from the proteins presented by any one of SEQ ID NOs: 1-82, or a homologue with at least 80% identity of any one thereof, or an engineered Ena protein. In a further specific embodiment, said Ena protein of said Ena-MBP fusion protein is defined as described in Remaut et al. (WO2022/029325A2), and further described and exemplified below. In another specific embodiment, said Ena protein of said Ena-MBP fusion protein relates to an S-Ena protein, or an L-Ena protein.

Another embodiment relates to said Ena-MBP fusion protein, wherein the MBP insert, optionally comprising an N- and/or C-terminally flanking linker, is connected to the Ena protein through linking the N-term of the insert to the C-term of the Ena which is cleaved or interrupted at an exposed loop region for insertion, and the C-term of the insert to the N-terminal end of the Ena interrupted at an exposed loop region. In an alternative embodiment, the Ena-MBP fusion protein is built from an engineered Ena protein wherein the loop sequence is (partially) removed and/or contains amino acid substitutions, preferably wherein the selected loop for insertion of the MBP is the surface exposed DE- or HI-loop of an Ena protein, in particular an S- or L-Ena protein. In particular, the DE- or HI-loop region as defined for Ena1A (SEQ ID NO:1), Ena1B ‘SEQ ID NO:8), exemplified and defined herein as the sequence corresponding to SEQ ID NO: 124 or 127 in Ena1B (SEQ ID NO:8), or SEQ ID NO:186 or 187 in Ena1A (SEQ ID NO: 1), or alternatively the superimposed corresponding loop of any one of the Ena sequences of SEQ ID NOs: 1-82, or an engineered Ena form thereof.

In a further specific embodiment, the Ena-MBP fusion protein as described herein comprises an MBP which specifically binds any one of the alkaline earth metals or transition metals or salts thereof, and/or any one of the metals listed herein as Be, Mg, Ca, Sr, Ba, Ra, Sc, Ti, V, Cr, Mn, Fe, Co, Ni, Cu, Zn, Y, Zr, Nb, Mo, Tc, Ru, Rh, Pd, Ag, Cd, Lu, Hf, Ta, W, Re, Os, Ir, Pt, Au, Hg, Se, Ag, As, Pb and/or salts thereof and/or comprises a MBP classified as iron-sulphur proteins, or as high potential iron-sulfur proteins (HiPIPs), or metallothionein, or a Bacillus thuringiensis subsp. Kurstaki MBP, as for instance exemplified herein.

In a further specific embodiment the Ena-MBP fusion as described herein relates to a fusion wherein the MBP insertion into the Ena protein subunit is obtained via genetic fusion and recombinant expression of the genetic fusion molecule. More specifically, said Ena-MBP fusion comprises an Ena protein which lacks the loop region for insertion exposed at the surface, such as the DE- or HI-loop, or wherein the DE-loop or HI-loop sequence of said Ena may be truncated by a maximum of 1, 2, 3, or four amino acids as compared to the wild type Ena protein, for inserting the MBP sequence, optionally comprising flanking linkers. A further specific embodiment relates to the Ena-MBP fusions wherein the insert sequence has been adapted as compared to the original MBP by addition of engineered DE- or HI-loop residues and optional linker residues, including alternative options as compared to the above linkers and loop insertions, though still allowing to be inserted into the Ena protein without interfering on the functionality or steric positioning of the Ena protein assembly, as outlined further herein.

Alternatively, said engineered Ena-MBP fusion protein may also be designed by a method using structure-guided identification candidate metal-binding polypeptides or fragments thereof to insert into an Ena protein, said method comprising the steps of:
a. Prediction or modelling of a protein 3D structure using a software program known to the skilled person, such as the Alphafold 2.0 program running on a computer,
b. Identifying the putative MBPs based on the predicted 3D structure of step a., wherein those polypeptides with at least 3 free, non-disulfide bonded cysteines are present in close proximity, for complexation of a metal ion,
c. Selection of those putative MBPs or fragments thereof, for which the 3D structure demonstrates that the N- and C-terminus of said MBP are at a distance from each other that is suitable to insert within the Ena protein its exposed loop at the proposed cleavage site, more specifically, preferably suitable to insert in the Ena sequence lacking the DE- or HI-loop as provided in SEQ ID NOs: 138 and 140, respectively, when cleaved at said residue where the loop is lacking, or similarly in a corresponding Ena homologue thereof,
wherein a suitable distance means that the N- and C-term of said MBP, optionally with flanking linkers and/or loop residues or substitutes, can be genetically fused to the Ena lacking said loop within said region lacking the original loop, resulting in a fused MBP that will bridge a distance of 10±2 angstrom between the Ena open ended loop structure, and
d. optionally, optimize the length of the sequence stretch surrounding the fusion sites, i.e. the short flanking linker(s), for instance by linker shuffling, truncating or adding flexible amino acids.

A further aspect of the invention relates to a multimer comprising one or more Ena proteins wherein at least one is an Ena-MBP fusion protein as described herein, wherein said Ena (fusion) proteins are present in the multimer as non-covalently linked subunits. In a specific embodiment, said multimer comprises at least seven, and/or maximally twelve, Ena protein subunits wherein at least one comprises an Ena-MBP fusion protein as described herein. Alternatively, said multimers may comprise identical Ena fusion proteins, or different Ena-MBP fusion proteins.

Another aspect relates to a protein fiber comprising at least 2 multimers as described herein, or preferably comprising at least 2 multimers comprising at least 7 Ena proteins, and/or maximally 12 Ena proteins, wherein at least one is an Ena-MBP fusion protein as described herein, wherein said multimers are longitudinally stacked and covalently linked through at least one disulphide bond.

Alternative aspects of the invention relate to modified surfaces comprising an Ena fusion protein or Ena-MBP fusion protein as described herein, or Ena fusion protein multimer or fiber as described herein. Alternatively, the Ena fusion as described herein is comprised in a complex with the metal ion or cofactor bound to said MBP of the Ena fusion protein. Also aspects of a chimeric gene comprising a nucleic acid molecule encoding the Ena fusion protein or a host cell comprising said chimeric gene or complex or Ena fusion protein or multimer or fiber, as described herein, are intended in certain embodiments.

A further aspect thus related to a method for producing said Ena-MBP fusion protein, multimer, or fiber, of the invention, comprising the steps of:
a. Recombinant introduction and expression of the chimeric gene in a host cell, or cultivating the host cell comprising said Ena fusion, and
b. Enriching or purifying the Ena fusion proteins in the form of monomers, multimers, or fibers from said cultivation medium or cell culture.

In a specific embodiment, the enrichment of the Ena-MBP-comprising assemblies in step b is obtained through mechanical cell lysis or chemical cell lysis, followed by enzymological digestion to resolve undesired host cell polymers, the latter preferably performed using a glycosylhydrolase, protease or nuclease, and/or incubation of the cell lysate and/or cultivation medium in a heated denaturing solution, and recovery of active or functional Ena fusion protein, multimer, or fiber by sedimentation or ultrafiltration. In a specific embodiment, said heated solution is at least 40° C. or more, and the denaturing conditions is for instance provided by the presence of 1 to 10% detergent sodium dodecyl sulphate (SDS) in said solution.

A final aspect relates to different uses of said Ena-MBP fusion containing protein assemblies as described herein, as bionanomaterials, for instance in metal mineralisation, sequestration, metal removal or exchange, which may be beneficial in waste water treatment, water softeners, and bioremediation purposes, among others. One embodiment specifically discloses the use of the Ena-MBP fusion protein, multimer or fiber wherein at least one or more Ena fusion proteins comprise a calcium-binding protein, preferably selected from any one of SEQ ID NOs: 157, 159-164, or a homologue with at least 90% identity thereof, in particular wherein the Ena fusion protein comprises any one of SEQ ID NOs: 158, 172-177.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1C. Engineerable Ena1B loops. (FIG. 1A) S-ENA oligomer, made of self-assembled covalently associated Ena1B protomers, shown in ribbon representation and transparent molecular surface representation. Solvent-accessible loops in the Ena1B protomers are coloured black for clarity. (FIG. 1B) Zoom in on the Ena1B solvent-accessible loops (a). The DE-loop and HI-loop are shown in stick representation. This figure was generated using PyMol®, using pdb entry 7A02. (FIG. 1C) Schematic representation of Ena1B fusion proteins with exogenous domains added into loop DE (Ena1B-DE-fusions) or loop HI (Ena1B-HI-fusions).

FIGS. 2A-2H. Structures of selected metal binding proteins. (FIG. 2A) Desulfovibrio vulgaris rubredoxin (pdb: 2KKD), (FIG. 2B) Rhodopseudomonas palustris TIE-1 PioC (PDB 7a4L), (FIG. 2C) B. thuringiensis cold-shock protein WP_098221117.1, (FIG. 2D) B. thuringiensis TKJ08955.1 hypothetical protein FC702_01375, (FIG. 2E) B. thuringiensis hypothetical protein WP_197262982.1, (FIG. 2F) B. thuringiensis hypothetical protein WP_000861196, (FIG. 2G) B. thuringiensis hypothetical protein WP_086405534.1, (FIG. 2H) B. thuringiensis zinc ribbon domain-containing protein WP_142338290.1. Distances separating the truncated N- and C-termini are shown in angstrom. N- and C-termini of the respective sequences were truncated to lay within a distance of 10±2 Å, compatible with the distance separating the insertion sites in Ena1B loops DE or HI (see FIG. 1, FIG. 25). Structures are shown in ribbon representation, and cysteine residues in the (predicted) metal binding site are shown as stick, while bound metals are shown as spheres. Figures were generated using PyMol®.

FIGS. 3A-3C. AlphaFold2.0 prediction of Ena1B-HI-Rubredoxin. (FIG. 3A) De novo structure prediction, using Alphafold2, of the Ena1B-HI-Rubredoxin fusion protein, where Desulfovibrio vulgaris rubredoxin is inserted into Ena1B HI loop. Alphafold2 predicts that both the Ena1B scaffold (light grey) and the Rubredoxin insertion (black, circled in dashed lines) are able to adopt their native fold. (FIG. 3B, FIG. 3C) To evaluate the anticipated self-assembling propensity of the Ena1B-HI-Rubredoxin fusion protein, the predicted structures of a minimal of three protomers are superimposed onto the S-ENA polymer to check for steric compatibility, i.e. lack of steric clashes laterally (FIG. 3B, top view) or longitudinally (FIG. 3C, side view). In case of clashes, linker sequences (L1 and L2) can be adjusted to alter the angle and distance of the insertion domain relative to the Ena1B fiber. Figures generated with PyMol®.

FIGS. 4A and 4B. Western blot of Ena1B fusion proteins. (FIG. 4A) Western blot of Ena1B-DE-HiPIP and Ena1B-HI-Rubredoxin. (FIG. 4B) Western blot of Ena1B, Ena1B-DE-Rubredoxin, Ena1B-HI-HiPIP, Ena1B-HI-WP_142338290.1, Ena1B-DE-WP_142338290.1, Ena1B-HI-WP_086405534.1, Ena1B-HI-WP_000861196, Ena1B-HI-WP_197262982.1 and Ena1B-DE-WP_197262982.1. Bands 10 kDa, 15 kDa, 40 kDa, 55 kDa, and 70 kDa of the PageRuler™ Prestained protein ladder are marked by an arrow. Protein bands are indicated by rectangular boxes. Control used for the Western blot was Ena1B.

FIGS. 5A-5H. Negative stain images of recombinant Ena1B with inserts in the DE-loop. (FIG. 5A) Ena-DE-Rubredoxin. (FIGS. 5B, 5C, and 5G), Ena1B-DE-HiPIP, (FIG. 5D) Ena1B-DE-WP_197262982.1, (FIG. 5E, FIG. 5H) Ena1B-DE-WP_142338290.1, (FIG. 5F) Ena1B. Fibers are indicated by black arrows. Aggregates are indicated by white arrows and unidentified filaments are indicated by grey arrows.

FIGS. 6A-6F. TEM images of recombinant Ena1B with inserts in the HI-loop. Fibers are indicated by black arrows. Aggregates are indicated by white arrows and spirals are indicated by grey arrows. (FIG. 6A) Ena-HI-Rubredoxin. (FIGS. 6B and 6C) Ena1B-HI-WP_098221117.1, (FIG. 6D) Ena1B-HI-WP_197262982.1, (FIG. 6E) Ena1B-HI-WP_086405534.1, (FIG. 6F) Ena1B-HI-WP_142338290.1.

FIGS. 7A and 7B. Image of Ena1B-DE-Rubredoxin. (FIG. 7A) TEM-image of Ena1B-DE-Rubredoxin. (FIG. 7B) TEM-image of Ena1B-DE-Rubredoxin twice zoomed. Fibers are shown by blue arrows, remnants of sodium dodecyl sulphate (SDS) by black arrows and spirals of Ena1B-DE-Rubredoxin by grey arrows.

FIGS. 8A and 8B. Image of Ena1B-HI-Rubredoxin (FIG. 8A) TEM image of Ena1B-HI-Rubredoxin. (FIG. 8B) 5× zoomed TEM-image of Ena1B-HI-Rubredoxin. Arrows indicate fibers.

FIGS. 9A and 9B. TEM images of FIG. 9A. Ena1B-HI-Rubredoxin. FIG. 9B. Ena1B fiber.

FIGS. 10A and 10B. Electrostatic charges of Ena1B-HI-Rubredoxin as shown in Cryo-EM. (FIG. 10A) Nearly constant distances between the fibers are indicated by arrows. (FIG. 10B) Electrostatic potential map of rubredoxin to indicate its negative charge on the surface. Figures generated with PyMol®.

FIGS. 11A and 11B. Comparison of 2D-class of (FIG. 11A) Ena1B fiber and (FIG. 11B) Ena1B-HI-rubredoxin fiber. arrows indicate the rubredoxin grafted onto Ena1B. 2D-classes are generated through CryoSparc®.

FIG. 12. Two classes of Ena1B-Rubredoxin-HI from TEM-images. Left figure depicts the thicker fiber (142.7 Å) while the right figure points to thinner fibers (134.7 Å). Distances between rubredoxins are indicated by double pointed arrows (16.7 Å and 20.2 Å). Images are generated with CryoSparc™.

FIGS. 13A and 13B. Top (FIG. 13A) and side view (FIG. 13B) of Ena1B-HI-Rubredoxin in density map. Model built Ena1B-HI-Rubredoxin is shown in ribbon representation and density map is shown in transparent surface representation. Figures were generated in ChimeraX®.

FIGS. 14A and 14B. Comparison of (FIG. 14A) Ena1B fiber and (FIG. 14B) Ena1B-HI-Rubredoxin fiber. The fibers are shown in ribbon representation. Picture is generated in PyMol®.

FIGS. 15A-15C. Subunits of Ena1B-HI-Rubredoxin and comparison with Ena1B. (FIG. 15A) Axial side view of subunits of Ena1B-HI-Rubredoxin. (FIG. 15B) Alignment of Ena1B (dark) and Ena1B-HI-Rubredoxin (light) monomer. (FIG. 15C) Zoom in the N-terminal connector (Ntc) of the aligned monomers. The distance between Cys11 and Lys16 are measured in Angstrom. Cysteines are shown in stick representation. Subunits are shown in ribbon representation. Figures are generated with PyMol®.

FIG. 16. X-ray Fluorescence spectrum of Ena1B fusion proteins. Ena1B-DE-WP_142338290.1 is shown in grey, Ena1B-HI-Rubredoxin is shown as dotted line, Ena1B-HI-Rubredoxin with nickel exchanged is depicted as thick line and Ena1B-DE-HiPIP is shown in grey thin line. Control was an Ena1B with an HA-tag in the HI-loop is dotted line.

FIG. 17. Western blot of recombinant fibers with and without the presence of beta-mercaptoethanol (BME). Bands 10 kDa, 15 kDa, 35 kDa, 40 kDa, 55 kDa, and 70 kDa of the PageRuler™ Prestained protein ladder are marked by arrows. monomers, trimers, tetramers and oligomeric Ena1B-HI-Rubredoxin can be seen in rectangles. The same can be seen for Ena-DE-HiPIP in rectangles.

FIGS. 18A and 18B. Heat-dependent denaturation of oxidized Ena1B-HI-Rubredoxin. (FIG. 18A) Absorption spectra of oxidized Ena1B-HI-Rubredoxin. Incubations of 25 min done in temperatures 25° C. (black), 80° C. (dark grey), 100° C. (grey), and 121° C.-autoclaved (light grey). The protein (1.23 mM) was buffered with 10 mM Tris pH 7. Blue arrows indicate the decreasing intensity of the absorption peaks at wavelengths 320-380 nm, 490 nm and 570 nm. (FIG. 18B) Loss of absorbance at 490 nm in percentage (0-1 normalized), and in function of temperatures in degrees Celsius. Graphs were generated with GraphPad Prism®.

FIGS. 19A AND 19B. Heat-dependent denaturation of reduced Ena1B-HI-Rubredoxin. (FIG. 19A) Absorption spectra of reduced Ena1B-HI-Rubredoxin fibers. Incubation of 30 min in temperatures 25° C. (grey), 100° C. (black), and autoclaved (light grey). The protein (1.21 mM) was buffered with 10 mM Tris pH 7 in nitrogen gas, and reduced with an excess (10 mM) of sodium dithionite. Arrows indicate the decreasing intensity of the absorption peaks at 310 nm and 350 nm. (FIG. 19B) Loss of absorbance at 490 nm in percentage (0-1 normalized), and in function of temperatures in degrees Celsius. Graphs were generated with GraphPad Prism®.

FIG. 20. Heat-denaturation curve of oxidized and reduced Ena1B-HI-Rubredoxin. Temperature-dependent denaturation of oxidized (black circles) and reduced (grey squares) Ena1B-HI-Rubredoxin incubated at increasing temperatures for 30 min and measured as absorbance at 490 nm. The oxidized protein (1.23 mM) was buffered with 10 mM Tris pH 7. The reduced protein (1.21 mM) was buffered with 10 mM Tris pH 7 and reduced with an excess (10 mM) of sodium dithionite. Graphs were generated with GraphPad Prism®.

FIG. 21. Absorption spectra of oxidized (black) and reduced (grey) Ena1B-HI-Rubredoxin. The protein was buffered with 10 mM Tris pH 7 and reduced with an excess of sodium dithionite (10 mM). Green arrow points to the peak obtained in the reduced spectrum (310 nm). The spectra were normalized by dividing the absorbances by absorbance at 280 nm. Graphs were generated with GraphPad Prism®.

FIGS. 22A-22C. Redox titration of Ena1B-HI-Rubredoxin and methylene blue. (FIG. 22A) Absorbance spectra of oxidized Ena1B-HI-Rubredoxin and methylene blue reduced with increasing concentrations of sodium dithionite (in rainbow colours red to purple, followed by brown, grey, black). (FIG. 22B) Zoomed view of graph in panel a in wavelength range from 400 nm to 700 nm and smoothed (2^{nd} order smoothing, 5 neighbours). Decrease of the absorbance peaks 490 nm and 668 nm are highlighted with blue arrows. (FIG. 22C) Absorbance at 490 nm (square) and 668 nm (trianglefi) plotted in function of the concentration of sodium dithionite. 23 spectra were measured. Graphs are obtained by GraphPad Prism®.

FIG. 23. Nernst plot of reduction of Ena1B-HI-Rubredoxin and methylene blue. A line fit is used to calculate the Y-intercept. Graphs were generated with GraphPad Prism®.

FIGS. 24A and 24B. Structural conservation of the ENA scaffold in WT Ena1B and Ena1B-HI-rubredoxin. (FIG. 24A) 3D cryoEM electron potential map of Ena1B-HI-rubredoxin fibers shows unambiguous density for the Ena1B scaffold, and the presence of the rubredoxin insertion in the HI loop. (FIG. 24B) Superimposition of the structures of Ena1B and Ena1B-HI-rubredoxin show the high structural conservation of the Ena1B scaffold also in presence of the folded rubredoxin domain in the HI loop. Highlighted in dashed circles are Leu98 and Glu104, the last residue of strand H and first residue of strand I, respectively. These residue form the structurally conserved boundaries or ‘anchor points’ for the Ena1B scaffold. Insertion sequences able to connect to these Ena1B HI loop anchor points with conservation of their relative spacing of 10±2 angstrom, possibly with the inclusion of a flexible linker sequence, are compatible with the Ena1B scaffold obtaining its native fold and self-assembling properties.

FIG. 25. Design principles of Ena1B fusion constructs. Ena1B sequences with the two permissive sites, referred to as DE loop and HI loop, demonstrated to allow insertion of exogenous sequences with the maintenance of the Ena1B folding characteristics and self-assembling properties. The DE loop and HI loops are flanked by, respectively, Val60 and Asp56, and Leu98 and Glu104, which form the last and first residue of the beta-strands connected by the respective loops. Insertion sequences that allow these anchor sites to adopt their native spacing of 10±2 Å are compatible with folding of the Ena1B scaffold. Domains to be inserted are designed as Linker1-Insert-Linker2. Linkers are added to adjust the distance between the N and C terminus of the insert to be compatible with opened Ena loops (10+/−2 Å), and can comprise any amino acid.

FIGS. 26A and 26B. Curlin and curlin-like repeats as insertion sequences for Ena1B fusion proteins. (FIG. 26A, FIG. 26B) sequence motif and structural models of curlin (cR; FIG. 26A) and curlin-like (cIR; FIG. 26B) repeats (Sleutel et al. 2022), where x1 to x7 are surface localized sites that can be any amino acid. Curlin and curlin like repeats can be inserted as single (cR1 and clR1) or double repeats (cR1-GGED-cR2 and clR1-GGG-clR2) using indicated L1 and L2 linker sequences. When x1 to x7 are replaced by Asp, this results in a regular spacing and positioning of the aspartic acids, resulting in a molecular surface able to bind and organize calcium carbonate salts, thereby adopting a calcite nucleating and thus biomineralizing activity. Examples of candidate calcite mineralizing inserts are shown for both cR and cIR, further herein.

FIGS. 27A and 27B. Fibers of Ena1B fusions with biomineralizing inserts diactinin and R4.5-2RfD. (FIG. 27A) TEM images of Ena1B-HI-diactinin (SEQ ID NO: 158) or Ena1B-HI-R4.5-2RfD (SEQ ID NO:173) fibers expressed in E. coli and isolated after cell lysis, 2 hour lysozyme digestion, and a subsequent 1 hour incubation in 1% SDS at 100° C. Ena1B fusion fibers are then isolated by sedimentation. (FIG. 27B) Pellet of Ena1B-HI-R4.5-2RfD fibers obtained from a 1 L E. coli culture grown overnight on LB. Cells are lysed and incubated with lysozyme for 2 hours to digest the peptidoglycan sacculus. Cell lysates are subsequently incubated for 1 hour in 1% SDS at 100° C. to dissolve cellular proteins and membranes. Intact Ena1B fusion fibers are then isolated by sedimentation, resulting in a pellet of Ena1B fusion fibers that makes up as much as 40-50% of the total cell mass.

FIGS. 28A-28D. Calcite nucleating activity of Ena1B-HI-diactinin and Ena1B-HI-R4.5-2RfD. (FIG. 28A, FIG. 28B, FIG. 28C, FIG. 28D) TEM images of WT Ena1B fibers (FIG. 28A), Ena1B-HI-diactinin fibers (FIG. 28B) or Ena1B-HI-R4.5-2RfD (FIG. 28C, FIG. 28D) incubated in a solution of hard water, containing 6.5 mM calcium carbonate. Ena1B fusions with diactinin or R4.5-2RfD, but not WT Ena1B, shown the presence of mineral deposits corresponding to calcium carbonate. Ena1B-HI-diactinin and Ena1B-HI-R4.5-2RfD thus acts as calcium carbonate mineralizing materials.

FIGS. 29A and 29B. Water softening capacity of Ena1B-HI-diactinin and Ena1B-HI-R4.5-2RfD. (FIG. 29A) Residual calcium ion concentration in hard water treated with WT Ena1B, or Ena1B-HI-diactinin and Ena1B-HI-R4.5-2RfD. (FIG. 29B) Addition of Ena1B-HI-diactinin and Ena1B-HI-R4.5-2RfD fusions, but not WT Ena1B, results in a rapid precipitation of fiber-calcite complexes (see FIG. 28), that are removed from solution by sedimentation. The residual concentration of bivalent metals is determined by titration with EDTA in presence of indicator dye eriochrome black.

FIGS. 30A-301. TEM images of recombinant Ena1B with inserts in the HI-loop. (FIG. 30A) Ena1B-HI-Rubredoxin. (FIG. 30B) Ena1B-HI-HiPIP, (FIG. 30C) Ena1B-HI-WP_098221117.1, (FIG. 30D) Ena1B-HI-WP_197262982.1, (FIG. 30E) Ena1B-HI-WP_000861196.1, (FIG. 30F) Ena1B-HI-WP_086405534.1, (FIG. 30G) Ena1B-HI-WP_142338290.1, (FIG. 30H) Ena1B-HI-1AQQ, (FIG. 30I) Ena1B-HI-2MRB. Fibers are indicated by black arrows. Aggregates are indicated by white arrows and spirals are indicated by grey arrows.

FIG. 31. TEM image of recombinant Ena1A with rubredoxin in the DE-loop. Fibers are indicated by black arrows.

FIG. 32. TEM image of recombinant Ena1B with a HA-tag in the DE-loop and rubredoxin in the HI-loop. Fibers are indicated by black arrows.

FIG. 33. TEM images of recombinant Ena1B with rubredoxin in the DE-loop. Ena1B-DE-rubredoxin with glycine linker. Fibers are indicated by black arrows.

FIG. 34. Negative stain TEM image showing S-Ena fibers expressed in Lactococcus lactis.

FIGS. 35A and 35B. Anterior and lateral view of Ena1A and Ena1A-Rubredoxin-DE proteins. (FIG. 35A) Ena1A protein with the DE-loop shown in stick representation; (FIG. 35B) Ena1A-Rubredoxin-DE fusion protein wherein Rubredoxin is inserted in Ena1A in the DE-loop, shown in cartoon representation, with in grey: Rubredoxin, yellow: linkers, blue: Ena1A monomer. This figure was generated using PyMol®.

### DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

The term “nucleic acid sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. It also includes known types of modifications, for example, methylation, “caps” substitution of one or more of the naturally occurring nucleotides with an analog. By “nucleic acid construct” it is meant a nucleic acid molecule that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. “Promoter region of a gene” or “regulatory element” as used here refers to a functional DNA sequence unit that, when operably linked to a coding sequence and possibly placed in the appropriate inducing conditions, is sufficient to promote transcription of said coding sequence. “Operably linked” refers to a juxtaposition wherein the components so described are in a relationship permitting them to function in their intended manner. A promoter sequence “operably linked” to a nucleic acid molecule that is a coding sequence is ligated in such a way that expression of the coding sequence is achieved under conditions compatible with the promoter sequence. “Gene” as used here includes both the promoter region of the gene as well as the coding sequence. It refers both to the genomic sequence (including possible introns) as well as to the cDNA derived from the spliced messenger, operably linked to a promoter sequence. The term “terminator” or “transcription termination signal” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another gene. With a “chimeric gene” or “chimeric construct” or “chimeric gene construct” is meant a recombinant nucleic acid sequence molecule in which a promoter or regulatory nucleic acid sequence is operatively linked to, or associated with, a nucleic acid sequence that codes for an mRNA, such that the promoter or regulatory nucleic acid sequence is able to regulate transcription or expression of the associated nucleic acid coding sequence. The regulatory nucleic acid sequence of the chimeric gene is not operatively linked to the associated nucleic acid sequence as found in nature, and may be heterologous to the encoding nucleic acid sequence molecule, meaning that its sequence is not present in nature in the same constellation as presented in the chimeric construct. More general, the term “heterologous” is defined herein as a sequence or molecule that is different in its origin.

The terms “protein”, “polypeptide”, and “peptide” are interchangeably used further herein to refer to a polymer of amino acid residues and to variants and synthetic analogues of the same. A monomeric or protomer is defined as a single polypeptide chain from amino-terminal end (also referred to herein as N-term or N-terminus or N-terminal end) to carboxy-terminal end (also referred to herein as C-term or C-terminus or C-terminal end). A “protein subunit” as used herein refers to a monomer or protomer, which may form part of a multimeric protein complex or assembly.

The terms “chimeric polypeptide”, “chimeric protein”, “chimer”, “fusion polypeptide”, “fusion protein”, are used interchangeably herein and refer to a protein that comprises at least two separate and distinct polypeptide components that may or preferably may not originate from the same protein. The term also refers to a non-naturally occurring molecule which means that it is man-made. The term “fused to”, and other grammatical equivalents, such as “covalently linked”, “connected”, “attached”, “ligated”, “conjugated”, and as specifically used herein ‘inserted in’ when referring to a chimeric or fusion polypeptide (as defined herein) refers to any chemical or recombinant mechanism for linking two or more polypeptide components. The fusion of the two or more polypeptide components may be a direct fusion of the sequences or it may be an indirect fusion, e.g. with intervening amino acid sequences or linker sequences, or chemical linkers. The fusion of amino acid residues or (poly) peptides to an Ena protein or insertion into an Ena protein sequence, or to another protein of interest as described herein, may be a covalent peptide bond, or also refer to a fusion obtained by chemical linking. The term “fused to”, as used herein, and interchangeably used herein as “connected to”, “conjugated to”, “ligated to” refers, in particular, to “genetic fusion”, e.g., by recombinant DNA technology, as well as to “chemical and/or enzymatic conjugation” resulting in a stable covalent link.

The term “molecular complex” or “complex” refers to a molecule associated with at least one other molecule, which may be a protein or a chemical entity. The term “associating with” refers to a condition of proximity between a chemical entity or compound, or portions thereof, and a binding pocket or binding site on a protein. The complex as used herein may also comprise further molecules of a different nature, such as for instance one or more metals or metal ions, which may be in complex with the Ena-MBP or with the MBP as being present in its metal center(s), as chelated, or in any other chemically possible form as known to the skilled person. As used herein, the term “protein complex” or “protein assembly” or “multimer” refers to a group of two or more associated macromolecules, whereby at least one of the macromolecules is a protein. A protein complex or assembly, as used herein, typically refers to binding or associations of macromolecules that can be formed under physiological conditions. Individual members of a protein complex, such as protein subunits or protomers, are linked by non-covalent or covalent interactions. “Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. The binding or association maybe non-covalent—wherein the juxtaposition is energetically favoured by for instance hydrogen bonding or van der Waals or electrostatic interactions- or it may be covalent, for instance by peptide or disulphide bonds.

It will be understood that a protein complex can be multimeric. Protein complex assembly can result in the formation of homo-multimeric or hetero-multimeric complexes. Moreover, interactions can be stable or transient. The term “multimer(s)”, “multimeric complex”, or “multimeric protein(s) or assemblies” comprises a plurality of identical or heterologous polypeptide monomers. Polypeptides can be capable of self-assembling into multimeric assemblies (i.e.: dimers, trimers, pentamers, hexamers, heptamers, octamers, etc.) formed from self-assembly of a plurality of a single polypeptide monomers (i.e., “homo-multimeric assemblies”) or from self-assembly of a plurality of different polypeptide monomers (i.e. “hetero-multimeric assemblies”). As used herein, a “plurality” means 2 or more. The multimeric assembly comprises 3, 4, 5, 6, 7, 8, 9, 10, 11, 12, or more polypeptide monomers. The multimeric assemblies can be used for any purpose and provide a way to develop a wide array of protein “nanomaterials.” In addition to the finite, cage-like or shell-like protein assemblies, they may be designed by choosing an appropriate target symmetric architecture. The monomers or protomers and/or multimeric assemblies of the invention can be used in the design of higher order assemblies, such as fibers, with the attendant advantages of hierarchical assembly. The resulting multimeric or fibrous assemblies are highly ordered materials with superior rigidity and monodispersity, and can be functional as a multimer or fiber itself, or form the basis of advanced functional materials, such as modified surfaces containing multimeric assemblies or fibers, and custom-designed molecular machines with wide-ranging applications. More specifically, a multimer as used herein refers to homo- or heteromultimeric protein complexes which are non-covalently associated with each other to form an arc, turn, ring or disc-like structure; and/or further modified to grow or develop into self-assembling or triggered formation of nanofibers. Said multimeric assemblies may contain Ena fusion proteins as defined herein, or Ena protein variants, mutant and/or engineered Ena proteins, such as Ena-MBP fusion protein exemplified and described herein, as well as other proteins that may associate to said Ena protein-based multimers, called engineered multimers, thereby expanding said multimer towards further modifications required for certain applications.

By “recombinant polypeptide” is meant a polypeptide made using recombinant techniques, i.e., through the expression of a recombinant or synthetic polynucleotide, which may be obtained in vitro and/or in a cellular context. When the chimeric polypeptide or fusion polypeptide or biologically active (i.e. functional) portion thereof is recombinantly produced, it is also preferably enriched, purified or substantially free of culture medium, i.e., the impurities represent less than about 20%, more preferably less than about 10%, and most preferably less than about 5% of the volume of the protein preparation. By “isolated” or “purified” is meant material that is substantially or essentially free from components that normally accompany it in its native state.

“Homologue”, “Homologues” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity. The percentage of amino acid identity as provided herein is preferably in view of a window of comparison corresponding to the total length of the native or natural wild-type protein, or of the specific amino acid sequence referred to.

The term “wild-type” refers to a gene or gene product isolated from a naturally occurring source, or included in a cell, cell line or organism. A wild-type gene or gene product is that which is most frequently observed in a population and is thus arbitrarily designed the “normal” or “wild-type” form of the gene or gene product a observed in nature. In contrast, the term “modified”, “engineered”, “mutant” or “variant” refers to a gene or gene product that displays modifications in sequence, post-translational modifications and/or functional properties (i.e., altered characteristics) when compared to the wild-type or naturally-occurring gene or gene product. A knock-out refers to a modified or mutant or deleted gene as to provide for non-functional gene product and/or function. It is noted that naturally occurring mutants or variants may be isolated; these are identified by the fact that they have altered characteristics when compared to the wild-type gene or gene product, and a different sequence as compared to the reference gene or protein.

### DETAILED DESCRIPTION

The present invention relates to the first attempt of engineering Ena proteins and multimeric assemblies thereof, wherein the Ena's are engineered as such that a functional folded metal-binding protein (MBP) or fragment thereof was successfully inserted, still allowing the Ena protein to correctly fold, preferably to self-assemble into multimers and fibers, and this in a similar manner as observed for the wild type Ena multimeric assemblies and fibers (as reported in Pradhan et al., 2021 and Remaut et al., WO2022/029325A2). The design and application of MBPs for insertion into exposed Ena loops has surprisingly allowed to recombinantly produce functional Ena proteins and multimers, not being sterically hindered in their self-assembly, retaining the MBP its metal-binding activity. Although the majority of inserted proteins for metal binding used herein are known in the art, the ability of the Ena-MBPs representing folded protein inserts to still form recombinant S-Ena multimeric assemblies and/or fibers is not straightforward, since when not chosen well, protein folding can also be destabilized by domain insertions (Aroul-Selvam et al., 2004; Geitner et al., 2013; Zoldák et al., 2009). Also, the ability to bind metal co-factors is not a given. The invention further provides for a method for designing said Ena-MBP fusions based on the use of 3D structural modelling or prediction software ran on a computer, as to aid a skilled person to match the structural requirements needed for a successful Ena-MBP fusion protein, as further defined herein, based on the protein sequences at hand, and/or by further modifying said sequences through linkers or elegant modifications (substitutions, mutations, additions or deletions of residues near the insertion site). Once an Ena-MBP fusion is successfully designed and produced, the recombinant or chimeric product is preferably used as a bionanomaterial in applications involving metal-binding or metal-removal activity, such as metal mineralization, metal sedimentation, metal removal or metal exchange, as used for instance in heavy metal bioremediation, waste water treatment or purification, or specifically in view of calcium-binding such as for a water softener.

### Ena Proteins

The present invention relates to Ena-MBP fusion protein assemblies applicable in several constellations as next-generation biomaterials.

In a first aspect, the invention relates to the novel fusions formed by Ena and a metal-binding protein, as further defined herein, wherein said fusions are formed by insertion of the MBP, optionally with flanking linkers, into an exposed loop of the Ena protein surface, as to self-assemble into an Ena-MBP fusion protein providing for a novel protein subunit with metal-binding activity through the MBP domain or fragment.

In the context of the present invention, ‘self-assembly’ refers to the spontaneous organization of molecules in ordered supramolecular structures thanks to their mutual non-covalent interactions without external control or template. The chemical and conformational structures of individual molecules carry the instructions of how these are assembled. The same or different molecules may constitute the building blocks of a molecular self-assembling system. Generally, interactions are established in a less ordered state, such as a solution, random coil, or disordered aggregate leading to an ordered final state, which can be a crystal or folded macromolecule, or a further assembly of macromolecules. The association of small molecules or proteins into well-ordered structures is driven by thermodynamic principles, thus, based on energy minimization. The interactions involved in the molecular assembly process are electrostatic, hydrophobic, hydrogen bonding, van der Waals interactions, aromatic stacking, and/or metal coordination. Although non-covalent and individually weak, these forces can generate highly stable assemblies and govern the shape and function of the final assembly (Lombardi et al., 2019). Said self-assembling protein subunits described herein, and called Ena fusion proteins herein, are capable of forming self-assembling multimers and/or protein fibers envisaged herein to be applied in different settings and biomaterials. The multimeric or fibrous assemblies can be obtained from the pre-existing components termed building blocks, or subunits, more specifically the isolated self-assembling Ena fusion proteins or as used interchangeably herein Ena-MBP fusion proteins, as described herein.

The Ena protein family is defined herein (as defined and integrated herein from Remaut et al., WO2022/029325A2) as a protein with self-assembling properties, which is characterized in its amino acid sequence as belonging to the PFAM13157 class, i.e. characterized by the presence of a DUF3992 domain in its sequence, and which further requires to match the 3D structural fold of an Ena protein, as presented herein, specifically the fold of Ena1B (with a sequence depicted in SEQ ID NO:8), with a highly significant similarity score, defined as a Dali Z-score of 6 or more, 6.5 or more, or preferably n/10-4 or more, wherein n is the number of amino acids of said protein sequence. Specifically, said self-assembling protein subunit is provided by the bacterially originating proteins comprising an amino acid sequence selected from the group of SEQ ID NOs: 1-82, representing the Ena protein sequences described also in Remaut et al. (WO2022/029325A2), or any prokaryotic homologue with at least 60%, or at least 70% or at least 80% or at least 90% identity of any one of the sequences of SEQ ID NO:1-82, wherein the % identity is calculated over the full length window of the sequence. So one embodiment relates to the isolated self-assembling protein comprising a DUF3992 domain, as determined by aligning to its Hidden Markov Model as depicted in Table 1 of Remaut et al. (WO2022/029325A2), and wherein said protein subunit has a 3D (predicted) fold matching the Ena1B structure with a fold similarity score of 6.5 or more, as defined herein, and wherein Ena1B corresponds to SEQ ID NO:8 and wherein the Ena1B reference structure corresponds to the coordinates as provided in PDB7A02.

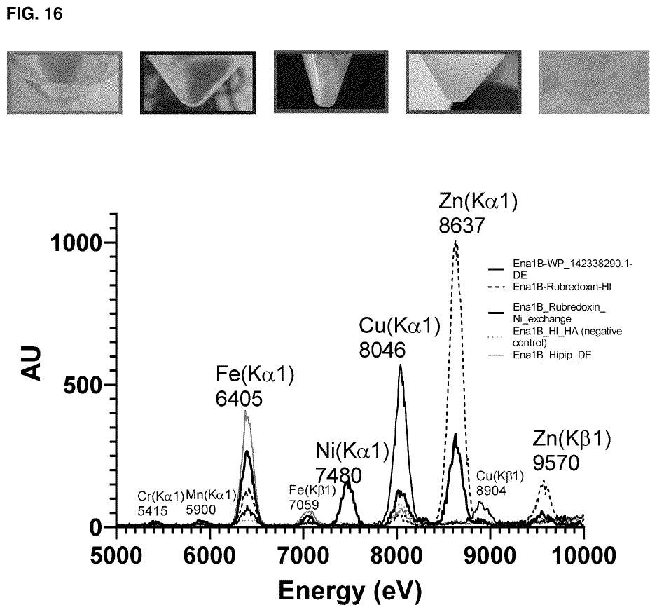

In a further embodiment, the Ena proteins referred to herein for using as part of the Ena fusion protein of the present invention, relates to said Ena protein family, as defined above, and/or as provided by the amino acid sequences depicted in SEQ ID NOs: 1-82, providing representative examples of the Bacillus Ena1A (SEQ ID NO: 1-7), Ena1B (SEQ ID NO: 8-14), Ena1C (SEQ ID NO: 15-20), Bacillus Ena2A (SEQ ID NO: 21-28, SEQ ID NO: 81), Ena2B (SEQ ID NO: 29-37), Ena2C (SEQ ID NO: 38-48, SEQ ID NO:82), and different types of other Bacillus Ena3 (SEQ ID NO: 49-80) proteins, respectively, or bacterial orthologues of any one thereof, which have at least 80% identity of any sequence depicted in SEQ ID NO:1-82. The regions and level of sequence conservation is shown for the Ena family members by the multiple sequence alignments depicted in FIGS. 16-19 of Remaut et al. (WO2022/029325A2). The bacterial DUF3992 domain-containing self-assembling proteins, such as those provided herein by SEQ ID NOs: 1-82, may be simply verified to fall under this Ena protein family by applying the present definition, i.e. by verifying whether a newly discovered protein is a member of this protein family, through a simple HMMR analysis (as provided for instance https://www.ebi.ac.uk/Tools/hmmer/and based on the matrix provided in Remaut et al., WO2022/029325A2) which allows the skilled person to define whether the protein comprises a DUF3992 domain, and compare its fold, which may be predicted simply based on the amino acid sequence, applying a structure matching tool, as known to the skilled person, and as exemplified herein, to assure the structure is provided as an Ena fold, i.e. having a matching fold with a Z score of at least 6.5 as compared to the Ena1B structure as provided in PDB7A02. Moreover, whether a protein with a DUF3992 domain has the propensity to self-assemble and appear as a multimer of at least seven, preferably six to twelve protein subunits, as claimed herein, may be determined by tests as known by the skilled person, for instance, but not limited to SDS-PAGE, dynamic light scattering analysis, size-exclusion chromatography, or preferably negative stain transmission electron microscopy.

More specifically, said Ena protein family has been identified herein as containing Ena1, Ena2 and Ena3 proteins, wherein Ena1 and Ena2 were each shown to contain 3 members (A, B, C), all comprising specific amino acid residue consensus motifs in their N- and C-terminal regions, allowing S-type fiber formation, and in addition a single Ena3A gene, required for L-type fiber formation. Finally, the Ena3A protein, encoded by an operon comprising a single Ena subunit in the Bacillus genome also comprises a DUF3992-domain, and has a conserved Cys residue pattern in its N-terminus, while its C-terminal region is more diversified from the Ena1/2 proteins, with Ena3A constituting the L-type fibers observed on Bacillus endospores. The L-type fibers appear as disc-like multimers which are longitudinally stacked via disulphide bonds for stabilizing the fiber. In a preferred embodiment, the Ena proteins referred to herein for using as part of the Ena-MBP fusion comprises an S- or L-Ena protein.

An “exposed loop” or “surface loop” of the Ena protein as referred to herein, or “loop region exposed on the Ena protein surface”, as used herein, refers to a region or polypeptide chain that is exposed at the surface of the self-assembled or folded protein. Specifically for Ena1B as defined by SEQ ID NO:8, and for which the 3D-structure is provided in PDB 7A02, examples of exposed loops are the DE or HI-loops, wherein said loops are respectively defined as the sequences provided by SEQ ID NO: 124 and SEQ ID NO: 127, corresponding to amino acids 55 to 59 and 99 to 103, resp. in SEQ ID NO:8. Alternatively, as further exemplified herein, for Ena1A (SEQ ID NO:1), the DE- and HI-loop are defined through structural homology and/or modelling based on Ena1B. So generally, determining those surface-exposed loop regions for other Ena proteins is performed for instance through resolving the 3D structure or through superimposing, modelling, or predicting the structure based on the Ena1B structure as to identify the corresponding amino acid residues or stretch for such exposed loop fragment wherein an insert can be engineered.

Moreover, other embodiment described herein relate to ‘modified’ or ‘engineered’ Ena protein subunits, or assemblies, as referred to herein, and are defined as also described in Remaut et al., WO2022/029325A2, wherein said engineered Ena proteins are being designed or derived from the existing (native) Enas obtained by changing the chemical composition, the length, and the directionality of interactions to create new units, or units with a new functionality, which contain all the necessary information that encodes their self-assembly. So with engineered Ena proteins is referred herein to a molecule comprising wild type Ena protein, for instance as provided in any one of SEQ ID NOs: 1-82, modified in any one of the following manners: by addition of further amino acid residues, such as tags, linkers, chimera or further polypeptides or chemical fusions; by substitution of one or more amino acid residues as compared to the wild type Ena, though retaining the typical Ena protein structure and function; by deleting a part of the wild type Ena protein sequence such as for instance a (partial) loop or unstructured number of amino acid residue; by insertion of one or more amino acid residues, preferably, as exemplified herein by insertion of folded polypeptides or fragments, wherein said insertion allow to retain the typical Ena structure as defined herein, so preferably with an insertion in a loop or sequence region where the 3D structure can be retained. An ‘engineered Ena protein’ as defined herein thus relates to non-naturally occurring forms of DUF3992-containing or Ena proteins, respectively, which is still capable of self-assembling and forming multimeric or fibrous structures. Engineered or modified or modulated proteins subunits or protein subunit variants, as interchangeably used herein, may show differences on their primary structural feature level, i.e. on their amino acid sequence as compared to the wild type (Ena) protein, as well as by other modifications, i.e. by chemical linkers or tags. An engineered protein subunit may thus concern a mutant protein, comprising for instance one or more amino acid substitutions, insertions or deletions, or a fusion protein, which may be a tagged or labelled protein, or a protein with an insertion within its sequence or its topology, or a protein formed by assembly of partial or split-Ena proteins, among other modifications. So in one embodiment, an engineered Ena protein is disclosed, wherein said engineered Ena protein is a modified Ena protein as compared to native Ena proteins, and is a non-naturally occurring protein. Non-limiting examples as provided herein relate to N- or C-terminally tagged Ena proteins, more specifically with a heterologous tag of at least 1, 2, 3, 4, 5, 6, 7, 8, 9, 10, 11, 12, 13, 14, 15 or more amino acid residues long, to acquire sterically frustrated Ena protein subunits for multimer formation without forming any fibrous assemblies; Ena mutant or variant proteins; Ena protein fusions or Ena proteins with a heterologous peptide or protein inserted within one of its exposed loops between β-strands, or Ena proteins formed upon assembly of Ena split-protein parts separately expressed in a host.

In a particular embodiment, the proteins subunit may be engineered Ena proteins comprising at least one Ena mutant or Ena variant protein subunit, or at least one engineered Ena which is an Ena-MBP as described herein. For example, though not-limiting, such Ena mutants or variants can be derived from the structural information demonstrating where modification or mutation of surface sidechains of the multimer or protein subunit is feasible. Furthermore, the Examples of engineered Ena fusions such as the Ena-MBP insertions of the present invention, provide for specific examples of insertion sites or insertion regions present in the 3D-structure as a loop exposed on the surface of an S- or L-Ena protein, and is, with reference to Ena1B (SEQ ID NO:8), defined by the positions located in/around the loops connecting the following β-strands, such as for the following stretches: the BC loop connecting B-C strands involving residues A30 to A33; the DE loop connecting D-E strands involving residues T55 to P59; the EF loop connecting E-F strands involving residues S66 to T72; and the HI loop connecting H-I strands involving residues G99 to A103. An insertion of a heterologous protein or peptide or linker in such a loop may consist of an amino acid sequence up to 400 residues long, and still retain the folding and structural features required for multimer formation. Besides the exemplified DE or HI-loop insertions as demonstrated herein, specifically how to create such an insertion variant or functional mutant engineered Ena protein may be envisaged as for example by modifying the primary amino acid sequence of for instance Ena1B as such: reordering the sequence by first inserting a single residue peptide or a (poly) peptide between β strands E and F by cleaving the Ena1B protein at residue S66, and adding the insert its N-terminal residue to the C-term of S66, and the insert its C-terminus to the N-term of G67 of Ena1B. An insertion may also be created by removing a number of amino acids from the loop of said Ena protein, for example the Ena1B sequence residues S66 to T72 may be replaced with an insert. The skilled person is aware of how to create similar inserts in different Ena protein loop areas as provided herein based on the disclosed structural features of the Ena proteins, and may also thereby create similar insertions for Ena homologues or engineered Ena protein forms thereof. With an ‘interrupted’ loop as used herein, is meant that the sequence of the loop of the native Ena protein is interrupted or the protein is cleaved (open-ended with a free N- and/or C-terminal end) to allow for insertion of another sequence or protein within said interrupted loop region.

### Ena Fusion Protein Assemblies

A second aspect of the invention relates to a protein multimer comprising or containing at least two, preferably at least more than two, three, four, five, six, seven or more of said self-assembling Ena or engineered Ena protein subunits, and preferably between 7 and maximally twelve subunits, which are non-covalently linked. More specifically, said multimer consists of seven, eight, nine, ten, eleven, twelve, 13, 14, 15, 16, 17, 18, 19, 20, or more self-assembling Ena protein subunits as defined herein, non-covalently stacked via β-sheet augmentation (a protein-protein interaction principle described in Remaut and Waksman, 2006). In a specific embodiment, said multimers as described herein may further comprise covalent connections, provided by for instance Cys connections between different protein subunits of said multimer (in suitable conditions). In one embodiment, said multimers are present ‘as such’, i.e. not as a filament or fiber constellation, and are therefore non-naturally occurring multimeric assemblies. Particularly, said self-assembling protein subunits defined herein as Ena proteins, May further comprise at least two conserved cysteine residues in their N-terminal region or N-terminal connector, as used interchangeably herein, for intermolecular disulphide bridge formation with further multimers. In a specific embodiment the multimeric assembly comprises seven to twelve protein subunits from the Ena protein family, or comprising engineered Ena proteins, specifically Ena fusion proteins, more specifically Ena-MBP fusion proteins, as described herein, wherein the Ena family may be provided by the amino acid sequences depicted in SEQ ID NOs: 1-82, providing representative examples of the Bacillus Ena1A (SEQ ID NO: 1-7), Ena1B (SEQ ID NO: 8-14), Ena1C (SEQ ID NO: 15-20), Bacillus Ena2A (SEQ ID NO: 21-28, SEQ ID NO:81), Ena2B (SEQ ID NO: 29-37), Ena2C (SEQ ID NO: 38-48, SEQ ID NO: 82), and different types of other Bacillus Ena3 (SEQ ID NO: 49-80) proteins respectively, or bacterial orthologues thereof, which have at least 80% identity of any sequence depicted in SEQ ID NO:1-82. A specific embodiment relates to said multimers with 7 to 12 protein subunits with identical self-assembling proteins as described herein. Alternatively, the multimers comprise at least 7 protein subunits wherein at least one of said protein subunits is an engineered self-assembling Ena protein, as defined herein and which concerns a non-naturally occurring Ena protein. Alternatively, said at least one engineered Ena protein subunit is an Ena-MBP fusion protein, or Ena-MBP fusion protein variant, or may be an Ena protein that is a fusion protein, or containing an inserted peptide or protein domain at exposed loops.

A specific embodiment relates to said multimers as described herein which are homomultimers or heteromultimers, and more specifically relate to multimers consisting of 6, or 7 to 12 subunits, and preferably relate to a heptamer, so consisting of 7 subunits, or a nonamer, so consisting of 9 subunits, both thereby possibly forming a disc-like multimer, or a decamer, undecamer or dodecamer, so consisting of 10, 11 or 12 subunits, respectively, thereby forming a helical turn or an arc of a β-propeller structure.

Overall, the those multimers as defined herein to comprise at least seven DUF3992 domain-containing protein subunits, which comprise at least one Ena fusion protein as defined herein, and wherein said protein subunits are non-covalently linked via β-sheet augmentation, with the aim to prevent further oligomerisation and covalent interaction triggered by the N-terminal and/or C-terminal regions forming inter-multimeric disulphide bridges, and/or to acquire additional functionalities or properties for said multimeric assemblies. Finally, the multimers as described herein provide for numerous applications in the field of next-generation biomaterials. In one embodiment, said multimers may be coupled to a solid surface, and as such provide for modified surfaces with properties of having an extreme resilient behaviour, thus being very stable and rigid materials.

The monomers or protomers and/or multimeric assemblies of the invention can be used in the design of higher order assemblies, such as protofibrils, fibrillar assemblies or fibrils and further fibers, with the attendant advantages of hierarchical assembly. The resulting multimeric or fibrous assemblies are highly ordered materials with superior rigidity and monodispersity, and can be functional as a multimer or fibrous structure itself, or form the basis of advanced functional materials, such as modified surfaces containing multimeric assemblies or fibrillar structures, and custom-designed molecular machines with wide-ranging applications.

The term ‘fiber’ as used herein may also be used interchangeably with the term ‘fibril’, ‘filament’, ‘fibrous assembly’, or ‘fibrous structure’, and refers to structured biochemical compounds, such as protein assemblies or protein-based assemblies, preferably composed of protein material, forming long-shaped ordered structures with diameters up to 100 nanometers, and potentially part of larger hierarchical structures. In view of the terminology used for defining fibers, ‘fibers’ may also be used as a term for as a potential plurality of nanofibrils, wherein fibers are generally considered to represent rather larger diameter (in the micro- to milli-scale) structures as compared to fibrils, and wherein ‘fibers’ thus preferably provide for a higher-ordered hierarchical structure of said plurality of fibrils. In a specific embodiment, such a fiber comprises a plurality of Ena (or Ena-MBP) nanofibrils, wherein each fibril makes further lateral associations to another fibril, to provide for a structured plurality of fibrils into a fiber. However, the term fiber as used herein refers rather to the protein nanofibrillar structure.

So, another aspect of the invention relates to protein fibers produced as to comprise at least two of said multimers as described herein, wherein said multimers comprise at least 7 Ena proteins and/or at least one engineered Ena protein which is an Ena-MBP-fusion protein, as described herein, or 7-12 subunits wherein said multimers are not hindered to longitudinally crosslink through disulphide bonds, more specifically through at least one disulphide bond, preferably two or more disulphide bonds. Said disulphide bonds may be formed between side chains of cysteine residues of the N-terminal region or N-terminal connector of one or more subunits of a multimer with one or more cysteine residues present in the N- and/or C-terminal region of one or more subunits of the multimer constituting the preceding layer of the longitudinally formed protein fiber. Said protein fiber may be a recombinantly produced fiber.

The protein fibers may thus be produced in a non-natural host, recombinantly, in cellulo and/or in vitro, and may comprise heteromeric or homomeric multimers. When heteromeric protein fibers are envisaged, the multimers may comprise one or more self-assembling (engineered) Ena proteins, or alternatively the protein subunits are identical except for that one or more subunit is an engineered protein form thereof, such as an Ena-MBP fusion. Homomultimeric protein fibers may be generated by recombinantly expressing a specific Ena protein or Ena protein mutant, variant or engineered Ena protein in a host cell. Any recombinantly produced protein fiber comprising one or more Ena protein subunits with at least one Ena-MBP fusion will thus represent a non-naturally occurring fiber.

In another embodiment, said protein fiber is an engineered protein fiber, comprising at least two multimers of which at least one multimer is an engineered multimer as defined herein, or wherein at least one multimer comprises at least one engineered Ena protein, as defined herein. In a preferred embodiment the protein fibers comprises multimers wherein the protein subunits comprise identical self-assembling Ena-MBP fusion protein subunits as described herein, and/or are composed of identical Ena proteins.

### Further Aspects

Another aspect of the invention relates to a chimeric gene construct comprising a promoter or regulatory sequence element that is operably linked to a DNA element comprising a coding sequence for the (engineered) self-assembling protein, preferably an Ena-MBP fusion protein, as defined herein. More specifically, said coding sequence may code for a protein comprising an Ena protein as depicted in SEQ ID NOs: 1-82, or a functional homologue of any of said Ena family members comprising Ena1/2A, Ena1/2B, Ena1/2C, or Ena3A, with at least 80% amino acid identity to any of SEQ ID NO:1-82, containing an insertion with an MBP protein or fragment, resulting in a sequence coding for an engineered Ena-MBP protein form thereof, as defined herein. In a specific embodiment, said promoter or regulatory element is heterologous to the coding sequence where it is operably linked to, and optionally is an inducible promoter, as known in the art.

A further embodiment relates to an “expression cassette” which comprises any nucleic acid construct capable of directing the expression of a gene/coding sequence of interest, which is operably linked to a promoter of the expression cassette, comprising the chimeric gene coding for said Ena-MBP protein as described herein. Expression cassettes are generally DNA constructs preferably including (5′ to 3′ in the direction of transcription): a promoter region, a polynucleotide sequence, homologue, variant or fragment thereof operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell. Such cassettes can be constructed into a “vector”. So a further embodiment relates to a recombinant vector comprising the chimeric gene, or expression cassette, comprising the sequence coding for the Ena-MBP fusion. The term “vector”, “vector construct,” “expression vector,” or “gene transfer vector,” as used herein, is intended to refer to a nucleic acid molecule capable of transporting another nucleic acid molecule to which it has been linked, and includes any vector known to the skilled person, including any suitable type. Vectors include, but are not limited to, plasmid vectors, cosmid vectors, phage vectors, such as lambda phage, viral vectors, such as adenoviral, AAV or baculoviral vectors, or artificial chromosome vectors such as bacterial artificial chromosomes (BAC), yeast artificial chromosomes (YAC), or P1 artificial chromosomes (PAC). Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Expression vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Suitable vectors have regulatory sequences, such as promoters, enhancers, terminator sequences, and the like as desired and according to a particular host organism (e.g. bacterial cell, yeast cell). Cloning vectors are generally used to engineer and amplify a certain desired DNA fragment and may lack functional sequences needed for expression of the desired DNA fragments. The construction of expression vectors for use in transfecting prokaryotic cells is also well known in the art, and thus can be accomplished via standard techniques (see, for example, Sambrook, et al. Molecular Cloning: A Laboratory Manual, 4th ed., Cold Spring Harbor Press, Plainsview, New York (2012); and Ausubel et al., Current Protocols in Molecular Biology (Supplement 114), John Wiley & Sons, New York (2016), for definitions and terms of the art.

A further embodiment relates to a host cell for expression of the chimeric gene as described herein, or for expression of the self-assembling protomers of the multimers or protein assemblies as described herein. In a specific embodiment this will possibly result in a host cell comprising the protomers or protein subunits of the multimers or forming the fibers comprising Ena-MBP fusion protein as described herein. ‘Host cells’ can be either prokaryotic or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. Recombinant host cells, in the present context, are those which have been genetically modified to contain an isolated DNA molecule, nucleic acid molecule or expression construct or vector of the invention. Representative host cells that may be used with the invention include, but are not limited to, bacterial cells, yeast cells, plant cells and animal cells. Bacterial host cells suitable for use with the invention include Escherichia spp. cells, Bacillus spp. cells, Streptomyces spp. cells, Erwinia spp. cells, Klebsiella spp. cells, Lactococcus spp., Serratia spp. cells, Pseudomonas spp. cells, and Salmonella spp. cells. Animal host cells suitable for use with the invention include insect cells and mammalian cells (most particularly derived from Chinese hamster (e.g. CHO), and human cell lines, such as HeLa. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarrowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. The host cells may be provided in suspension or flask cultures, tissue cultures, organ cultures and the like. Alternatively, the host cells may also be transgenic animals.

In a further aspect of the invention a modified surface or solid support is provided, said surface comprising an Ena protein, a multimer assembly, or a protein fiber as described herein, or an engineered form of any thereof. Said modified surface is composed by covalent attachments of said Ena protein, multimer or fiber to said surface, and may be a cellular or artificial surface, in particular a solid surface of any material type. Said modified surface may thus be used as a nucleator for epitaxial growth of a protein fiber, for instance when said modified surface is exposed or contacted with a solution of Ena proteins, wherein said Ena proteins are preferably present in monomeric or oligomeric form.

### Methods for Recombinant Production of Ena-MBP Fusion Proteins

Another aspect of the invention relates to the production method to recombinantly express the Ena-MBP protein as described herein, more particularly the Ena-MBP fusion proteins, multimeric and fibrous assemblies, as described herein, wherein said production is performed in vitro or in vivo/in cellulo, as described in Remaut et al., WO2022029325A2.

A specific embodiment describes a method to produce a Ena-MBP fusion protein monomers, or multimers, as described herein comprising the steps of:
a) expressing a chimeric gene construct for expression of the Ena-MBP fusion protein, as described herein, in a host cell, or using the host cell as described herein, wherein the self-assembling Ena-MBP protein subunit optionally comprises an N- and/or C-terminal tag, and (optionally)
b) purifying the self-assembled Ena proteins or multimers with an MBP insert, in particular the Ena-MBP proteins and/or Ena protein, the multimers being formed after oligomerisation of the expressed protein subunits.

Another embodiment relates to a method to produce a protein fiber as described herein, comprising the steps a) and b) of the above method, wherein the N- and/or C-terminal tag is a present as a removable or cleavable tag on said Ena and/or Ena-MBP fusion protein, said method further comprising the step c) wherein the N- and/or C-terminal tag is removed or cleaved off to allow further self-assembly of the formed multimers into protein fibers, thereby defined as the in vitro production method. Alternatively step c) may be exerted prior to the purification step b). Furthermore, a method is provided to produce the modified surface as described herein, comprising the steps a), b), and/or c) (or vice versa c) and/or b)), further comprising step d) wherein a surface is modified by displaying or covalently attaching the (engineered) Ena-MBP protein, multimer or fiber to said surface. Finally, the protein assemblies, such as fibers as described herein, may be produced within a cell, as depicted in the method for recombinant production of the Ena protein fibers comprising the steps of:
a) expressing the chimeric gene construct as described herein in a host cell, or using the host cell as described herein, or expressing an engineered Ena protein, or Ena-MBP protein, as described herein, wherein the protein subunit does not have a steric block or N-terminal tag, so the self-assembling protein consisting of a engineered self-assembling Ena protein with a free N-terminal connector, and (optionally)
b) isolation of the Ena-MBP protein assemblies, such as fiber or multimers, formed after oligomerisation of the expressed protein subunits within the cytoplasm.

As such, the fibers are formed in the cytoplasm which allows to easily produce S-type or L-type like fibers in vivo, depending on the Ena that is expressed.

Another embodiment relates to said method wherein the purification in step b) comprises the steps of isolation by lysis of the cells, and/or solubilization of inclusion bodies, refolding of solubilized protein subunits, and purification of refolded protein multimers; alternatively by denaturing through heating and addition of 1-10% SDS containing solution. Further purification methods for instance using affinity chromatography, ion exchange chromatography, gel filtration, or further alternatives are known to the skilled person.

Further embodiments relate to producing the Ena-MBP fusion protein in a host cell, preferably according to the above method for recombinant production, involving introduction of a chimeric gene encoding said Ena-MBP protein in said host cell, or providing a host cell wherein said Ena-MBP protein is present. “Host cells” can be either prokaryotic or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. For all standard techniques see, for example, Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016). Recombinant host cells, in the present context, are those which have been genetically modified to contain an isolated DNA molecule, nucleic acid molecule or expression construct or vector of the invention. The DNA can be introduced by any means known to the art which are appropriate for the particular type of cell, including without limitation, transformation, lipofection, electroporation or viral mediated transduction. A DNA construct capable of enabling the expression of the chimeric protein of the invention can be easily prepared by the art-known techniques such as cloning, hybridization screening and Polymerase Chain Reaction (PCR). Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (2012), Wu (ed.) (1993) and Ausubel et al. (2016). Representative host cells that may be used with the invention include, but are not limited to, bacterial cells, yeast cells, plant cells and animal cells. Bacterial host cells suitable for use with the invention include Escherichia spp. cells, Bacillus spp. cells, Streptomyces spp. cells, Erwinia spp. cells, Klebsiella spp. cells, Serratia spp. cells, Pseudomonas spp. cells, Lactococcus spp. cells, Lactobacillus spp. cells, and Salmonella spp. cells. Animal host cells suitable for use with the invention include insect cells and mammalian cells (most particularly derived from Chinese hamster (e.g. CHO), and human cell lines, such as HeLa. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarrowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. The host cells may be provided in suspension or flask cultures, tissue cultures, organ cultures and the like. Alternatively, the host cells may also be transgenic animals.

### Metal-Binding Activity and Uses

Finally, there are numerous applications as touched upon already herein for said Ena-MBP fusion proteins or engineered Ena protein subunit-derived assemblies as next-generation biomaterials in the fields of metal-mineralisation, sequestration and bioremediation. The complex-formation with metals present in the environment is applicable as a stoichiometric metal binding capacity, with clear advantages in chelation and metal-ion cofactor activity. Alternatively, as indicated herein, the metal-binding activity may also act in a manner wherein nucleation of crystals or salt-derivatives containing the metal is induced, leading to sequestration and the option to sediment such complexes from a solution, such as waste or drinking water. Finally, the addition of calcium-rich or metal-rich solutions to such Ca-/metal-binding Ena fusion proteins may reversely lead to other potential applications to use the CaCO₃ or metal-complexes complexes in a manner wherein solidification, for use (e.g. as hardened biomaterial) or for removal (e.g. Cd heavy metal sedimentation) purposes, is desired.

The application of said Ena-MBP-containing fibers as biodegradable material will dictate the type of metal-binding protein to be inserted. As non-limiting examples disclosed herein, several metal-binding proteins were genetically fused, and Ena-MBP-containing fibers retained their capacity to spontaneously fold, besides the observation that metal-binding properties of the MBP were also observed within the fibrous assemblies. The selection of smaller (up to 6 kDa) insertion proteins is an advantage to maintain folding and avoid steric hindrance, though also somewhat larger folded proteins up to 20 kDa or larger seemed still usable, indicating that optimization and structural insights are useful to further explore the boundaries of the insertion potential. Moreover, when heteromultimers or fibers with a mixture of multimers containing Ena and Ena-MBP fusions are used during production and assembly, so that fewer MBP proteins are displayed on the surface, further options for enlarging protein inserts may be possible.

Finally, in one embodiment, a double insert was made into a single Ena protein, wherein the DE and HI loop regions were used for inserting an MBP, and a hemagglutinin tag. Upon recombinant expression, fibers were self-assembled, providing evidence that both exposed regions can be simultaneously engineered. So one embodiment relates to an Ena-MBP fusion protein as described herein wherein at least one additional (poly) peptide is linked or fused or inserted.

It is to be understood that although particular embodiments, specific configurations as well as materials and/or molecules, have been discussed herein for methods, and products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Example 1. Ena1B Loop Engineering

To investigate the engineerability of Ena1B (SEQ ID NO:8) as a proof of concept to explore the possibility to generate ENA-MBP fibers, metalloproteins or metal-binding proteins were grafted onto the surface of the appendage protein.

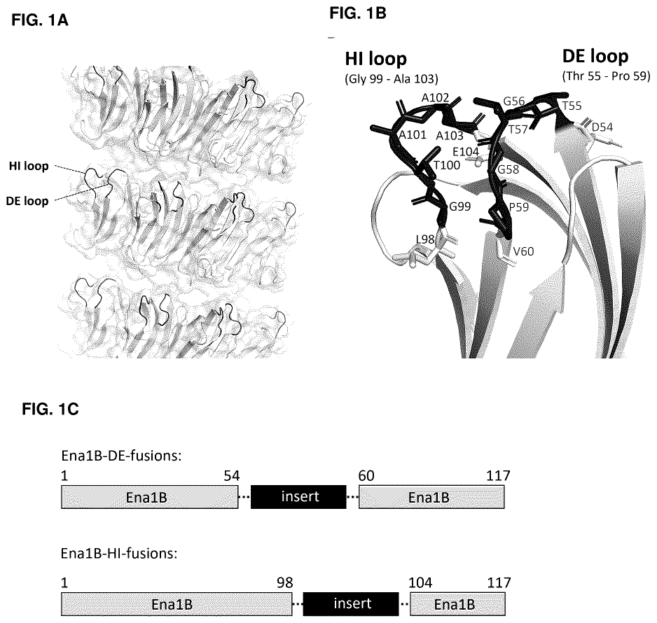

The insertion was tested herein to fit into at least one of the loops on the surface of Ena1B. With the loops facing outward of the Ena1B fiber (FIG. 1), this could enable redox activity, and moreover electron transport in a helix around the Ena1B fiber. As known from the literature, most insertions are done in loop regions (Edwards et al., 2008; Scalley-Kim et al., 2003; Senthil et al., 2019), since these take up a less important structural role in protein folding and are more tolerant of insertion, deletion, or substitutions. As such, a molecule can often be inserted with minimal effects on the stability and function of the main domain (Scalley-Kim et al., 2003). Surface loops are also more permissive to mutations and insertions (C.-S. Kim et al., 2009; Li et al., 2007). Potential substitution and insertion sites were previously investigated by inserting small 8-residue tags into surface-exposed Ena1B loops, pointing to the engineerability of at least the DE-loop and HI-loop (Remaut et al., WO2022029325A2). This, however, does not guarantee a successful insertion of peptides bigger than the inserted tags, i.e., that Ena proteins can still retain their self-assembling properties and that inserted molecules can attain their native fold. Indeed, when not chosen well, protein folding can also be destabilized by domain insertions (Aroul-Selvam et al., 2004; Geitner et al., 2013; Zoldák et al., 2009). Hence, the engineerability of the Ena DE-loop and HI-loop was further investigated by inserting small metal binding proteins. Variations of insertions in various parts of the loop were explored using AlphaFold 2.0 (AF2; Jumper et al., 2021; Tunyasuvunakool, et al. 2021). As the Ena1B DE-loop contains the sequences TGTGP (FIG. 1b), variations of the DE-loop played at openings in TGTG|P or TGT|GP. These variations were chosen as distally from the fiber as possible without hindering the proline as well the beta-strands flanking the insertion site. One point to consider was whether these variations would result in steric clashes formed within the self-assembled fiber, more specifically the subunits located above the monomer. Literature states that proline in loops function as a director for productive folding routes (Krieger et al., 2005). Based on the available structure we hypothesized that proline could be pushing TGTG and inserted sequences into the axial part of the Ena1B fiber (Krieger et al., 2005). Due to this, openings were also explored with deletion of the proline (TGTG| or TGT|G) (FIG. 1b). Ultimately, TGTG| would risk influencing the beta-strand as the insertion would be between the beta strand and the loop. TGT|G still risks an insertion that would not be distal enough, causing a clash with the neighbouring subunits (FIG. 1). To ensure that insertion happened as distally possible from the fiber without influencing the beta-strand, opening the DE-loop was finally done by substituting proline for glycine, which is a hugely flexible amino acid (Senthil et al., 2019). This generated an insert between TGTG| G.

The HI-loop of Ena1B (SEQ ID NO:8) consists of the sequence GTAAA (FIG. 1b). Two variations of opening the loop that were considered were G|TAAA and GT|AAA. Based on inspection of the structure, GT|AAA was chosen. It was expected this opening was still distal enough to ensure no steric clashes of the insertion with monomers above would occur (FIG. 1). To allow the most flexibility between the opening of the HI-loop and the insert, threonine and alanine directly flanking the insertion site were substituted for glycine. This residue was chosen as it is the most flexible amino acid due to lack of sidechains (Senthil et al., 2019). Thus, opening the HI-loop was finally done by substituting residues threonine and alanine to two glycine's, creating an opening at GG|GAA.

### Example 2. Metal-Binding Proteins (MBPs) to be Inserted in the Ena1B Scaffold

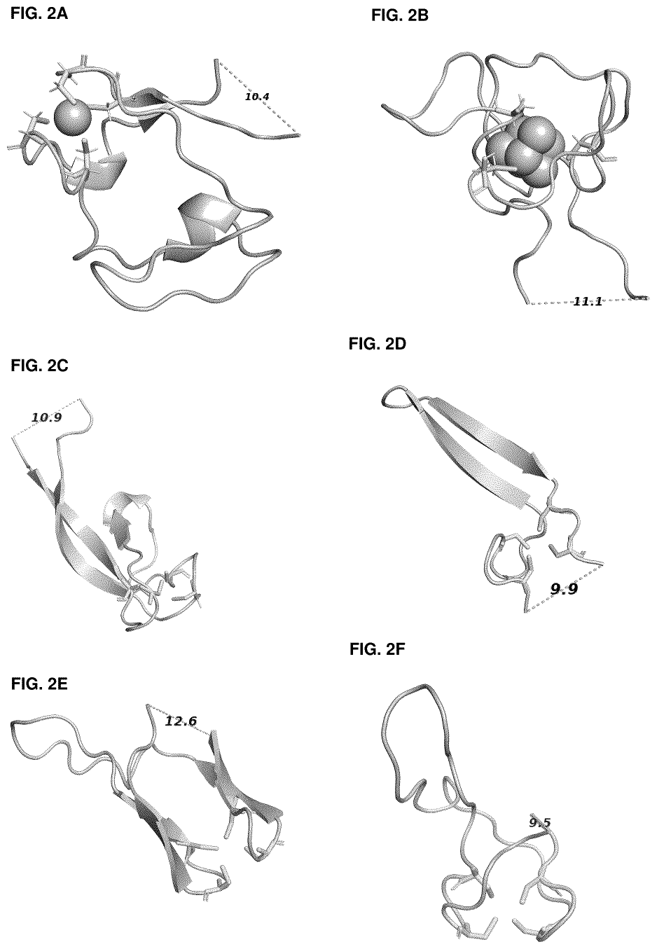

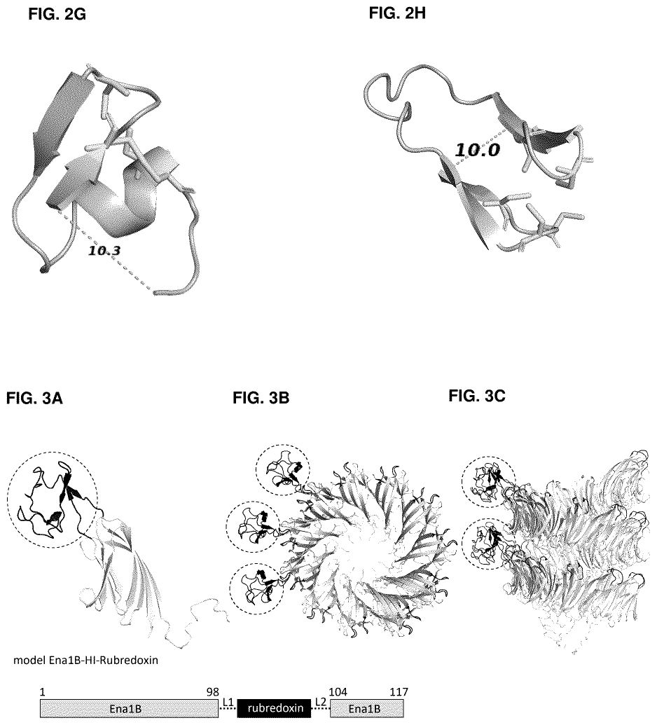

At first instance metal-binding proteins, and in particular electron transfer proteins, suitable to be inserted in the Ena1B loops had to be identified. The size of the selected electron transfer proteins was strategically limited to 6 kDa due to two reasons: to minimize the risk of steric hindrance with the subunit, and to minimize the conformational influence of the metalloprotein on the folding of Ena1B subunits. Furthermore, the viability of a domain insert is likely not only determined by the domain's small size, but also by adjacent or close termini (Aroul-Selvam et al., 2004). A short distance between the domain termini reinstates the protein conformational flexibility by imitating an inter-domain link which is distinguished among sequentially ordered domains (Aroul-Selvam et al., 2004). It also avoids destabilization of either domains that are linked together (Zoldák et al., 2009). So, we focused on (putative) metalloproteins where the N- and C-terminus are (predicted to be) as close as possible. As non-limiting examples, three types of electron-transfer proteins were tested to fit these conditions: iron-sulphur proteins (rubredoxins, FIG. 2a)), high potential iron-sulfur proteins (HiPIP's, FIG. 2b)), and a number of (putative) metal binding proteins of Bacillus thuringiensis subsp. Kurstaki (FIG. 2c-h).

The group of (putative) metal binding proteins of B. thuringiensis subsp. Kurstaki was identified herein based on the predicted localization of cysteine residues. To do so, the structures of small B. thuringiensis subsp. Kurstaki proteins were predicted using AlphaFold 2.0 (AF2) (Jumper et al., 2021). Selected MBPs have a putative metal binding site in which four cysteines were located in close proximity. To be selected as candidate MBPs for insertion into ENA, the structural library of the MPBs was manually inspected for proximity of the N- and C-terminus, a prerequisite for the insertion into a loop connecting two beta-strands. For some of the selected candidates, N- and/or C-terminal truncates were designed to lessen the distance between the N- and C-terminus, thereby ensuring compatibility with the receiving ENA loop (FIG. 2). Functional annotation of the target sequences was done through BLASTp against the Uniprot reference database.

### Example 3. Prediction of Candidate Ena1B-MBP Fusion Proteins for Self-Assembly into Fibers

To ensure that the metal-binding proteins fit well into the Ena1B fiber without hindering the formation of the endospore appendages, different flexible linkers lengths between the inserts and Ena1B were tested to identify the optimized length. Variations between 0-3 glycines before and after the insert were considered, predicted by Alphafold 2.0, then manually filtered.

Alphafold 2.0 (Jumper et al., 2021; Varadi et al., 2022) is an artificial intelligence program that predicts protein structures from amino acid sequences with a per-residue estimate of its confidence (pLDDT). The pLDDT scores from 0-100. Highly accurate predictions are scored at 100-90 and moderately accurate models score at 70-90. While low accurate models between 50-70 are analysed with caution, regions with scores below 50 are unstructured and should not be interpreted. Although the software is not designed or validated for the prediction of synthetically concatenated protein sequences like Ena1B-MPB insertion sequences, our finding was that the program predicted both the Ena1B scaffold and the inserted sequences with high accuracy. The accuracy of the predicted linker sequences is unclear but could still be used as a rough estimate of the chances for steric incompatibilities.

Final designs for inserts for the DE-loop and HI-loop of the Ena1B protein (SEQ ID NO:8) are as shown in Table 1 which were based on final predicted structures of the fusions taking into account the accuracy of predicted Ena1B-MBP fusion proteins. The Ena1B monomer in each prediction Ena1B-MBP fusion is predicted with a high accuracy, depicted by the colour red in AlphaFold 2.0 (AlphaFold 2.0 prediction confidence is coloured red to blue from high to low in PyMol® (The PyMOL Molecular Graphics System, Version 2.0 Schrödinger, LLC.). Linkers are coloured green to blue indicating low confidence to unstructured prediction between the Ena1B monomer and the MBP, which is normal for flexible linkers. Kinks were observed though, depicting an excess in amino acids, which were added to ensure the axial direction of the insert. In consideration of the folding of the insert, an excess in amino acids rather than a lack thereof seems favourable. Except for Ena1B-DE-WP_000861196, AF2 predicted the structure of the fusion proteins with high reported confidence levels, shown by red, orange to yellow colour of the proteins. In the case of WP_000861196, a blue colour indicating lack of confidence in the predicted 3D structure was visible, suggesting the insert was not properly folded. In this latter case, it is not clear if this reflects a true steric incompatibility of the fusion construct or an inability of AF2 to predict this non-natural sequence. Looking at the prediction of Ena1B-DE-WP_197262982.1, the second monomer collides with the supposed third monomer in the Ena1B fiber, which might be possible due to the direction WP_197262982.1 faces. Ena1B-DE-WP_197262982.1 aligned with Ena1B fiber showed that the distance between the monomers is greater through steric hindrances that could have been caused between adjacent monomers at a smaller distance. The succeeding monomer is thus repositioned. However, as the linkers are not confidently predicted, it could still possible that observed inserts would face distally from the fiber and the construct was still retained for in vitro expression as Ena1B fusion construct. With alignments of the inserts with the Ena1B fiber, monomeric structures do not seem to clash with the Ena1B fiber axially or distally. Therefore, remaining predicted AF2 constructs confidently assumed structures to create fibers. Results of all predicted constructs were therefore used in the next steps.

Next, the Ena1B fusion constructs with the MBP proteins inserted in the HI loop were predicted using AlphaFold 2.0 (AF2). The Ena1B monomer was predicted with high confidence as shown by the red colour. Linkers are coloured yellow to blue in structures suggesting the low confidence of the predictions between the Ena1B monomer and the metal binding protein (MBP). As linkers are flexible, this was not perceived to pose a problem. Comparing MBPs and MBPs grafted on the Ena1B monomer, the predicted 3D structures are nearly identical, except for Ena1B-DE-WP_000861196. This was also hinted by the blue colour, indicating a low confidence, unstructured prediction. Overall, the 3D-structure of all constructs indicated that the length of the linkers was sufficient for all inserts to be folded while attached to Ena1B.

Aligning a predicted Ena1B-HI-rubredoxin trimer with the Ena1B fiber showed that the Ena1B monomer positions are nearly identical, and rubredoxin faces the distal side of the fiber (FIG. 3). This was the same for the other insertions in the HI-loop. The AlphaFold 2.0 structure predictions for all Ena1B-HI fusion proteins suggest a high possibility of creating recombinant fibers without sterically clashing the Ena1B fiber axially or distally.

Moreover, as explained and used herein, the AF2-based prediction provides for a method to design potential Ena-MBPs, however taking into account that these provide candidate designs that are still subject to experimental verification, though this method has been shown herein to increase the chances of success. The program is trained to produce a multiple sequence alignment (MSA) through a neural network based on the inputted amino acid sequence (matrix 1). The distance and torsion angles between each pair of protein residues are put in a second matrix. The two matrices translate into an initial 3D structure which is then iteratively refined (Jumper et al., 2021). While producing accurate models in conditions where native structures with a known amino acid sequence have been solved, it is not yet able to capture the dynamics of newly synthetized proteins (Fowler and Williamson, 2022). More specifically, the relative orientations of different domains cannot be predicted from an MSA, which causes the low confidence to unstructured bridge between domains (e.g. FIG. 3). Secondly, AF2 does not evaluate how a protein reaches its folded state (Strodel, 2021). Through this, possible important energy barriers to reach the folded state of a synthetic protein can be overlooked. This can cause for inconsistency between a predicted structure and its actual structure. While AF2 is the currently best at predicting structures with high accuracies, important to keep in mind is that it does not prove to be the best solution. Rather, successfully predicted structures can serve as an estimation that can further be refined with experimental work such as the interpretation of cryogenic electron microscopy maps.

TABLE 1
Sequences for the different designed Ena-MBPs.

| | | SEQ |
|---|---|---|
| | | ID |
| Insert | Sequence as an insert | NO: |
| Rubredoxin | [symbol] KYVCTVCGYEYDPAEGDPDN | 129 |
| (5.44 kDa) | GVKPGTSFDDLPADWVCPVCG | |
| | APKSEFEAA | |
| | DE: TGTG-G-insert-G/R | |
| | HI: GG-G-insert-GAA | |
| HiPIP | VTKKASHKDAGYQESPNGAK | 130 |
| (5.87 kDa) | RCGTCRQFRPPSSCITVESP | |
| | ISENGWCRLYAGKA | |
| | DE: TGTG-insert-G/R | |
| | HI: GG-insert-GAA | |
| WP_098221117.1 | [symbol] EQTPVWECESEDCLGWMR | 131 |
| (5.66 kDa) | KNFSFEEEPKCPLCKSSMKS | |
| | GERLL [symbol] | |
| | DE: TGTG-insert-G/R | |
| | HI: GG-insert-GAA | |
| FC702_01375 | CVLCRDKGIIRKEIYPGVAL | 132 |
| (2.98 kDa) | TEGCNCE | |
| | DE: TGTG-G-insert-G-G/R | |
| | HI: GG-G-insert-G-GAA | |
| WP_197262982.1 | VVICPNCQSSAEIIHVLTAQ | 133 |
| (4.49 kDa) | SNQNVIYTCQVCHFVIRNIE | |
| | DE: TGTG-G-insert-SG-G/R | |
| | HI: GG-G-insert-SG-GAA | |
| WP_000861196 | KLCFYCFKIQSKKTKIIGDA | 134 |
| (4.14 kDa) | EGRMQICETCHKEL [symbol] | |
| | DE: TGTG-SA-insert-G-G/R | |
| | HI: GG-SA-insert-G-GAA | |
| WP_086405534.1 | LVCPKCGSAQARSGGYQECC | 135 |
| (2.57 kDa) | KIRL | |
| | DE: TGTG-A-insert-G/R | |
| | HI: GG-A-insert-GAA | |
| WP_142338290.1 | QLCSCCGYQNKDVKNLNLRE | 136 |
| (3.67 kDa) | WDCPSCRTHHD | |
| | DE: TGTG-AS-insert-G/R | |
| | HI: GG-AS-insert-GAA | |

Molecular weights of inserts are provided in brackets in the first column. Final designs as how to be inserted in the DE- or HI-loop of Ena1B resp., are shown by the flanking amino acids as present in the Ena1B loop, followed by a linker (italic font and underlined) and an ‘insert’ sequence. Deletion of amino acids from the original or wild type protein sequences are indicated by a strikethrough through in the sequence of the synthetic insert.

### Example 4. Cloning, Expression, and Purification of Ena1B-MBP Fusion Proteins

For the selected designs, the Ena1B fusion proteins were recombinantly expressed and analyzed for their functionality in complex formation with metal ions, and self-assembly into recombinant fibers. The methodology for insertion was the same for the DE-loop and HI-loop of Ena1B, using outward PCR using primers DE_open_f and DE_open_r (Table 5; SEQ ID NOs: 84-85), and linearization of the pET28a_Ena1B_no_his vector to open the DE/HI-loop. For the DE loop, as the last residue (proline) in the loop was not present in our designs, primers to linearize the vector excluded it. Overhangs with the loop were added to the inserts through PCR. These overhangs consisted of at least 15 bases complementary to the ends of the linearized vector and a linker consisting of DNA encoding one or two flexible amino acids. Inserts and vector were transformed into CaCl₂) competent E. coli TOP10 cells. For the HI-loop, threonine and its succeeding alanine were cleaved from the loop, therefore primers to open the vector were made as such that the amino acids glycine and both alanine's remain on the linearized vector. With a second set of primers, overhangs and linkers of the HI-loop were added to inserts through PCR. Inserts and vector were transformed into CaCl₂) competent E. coli TOP10 cells. Primer sequences are shown in Table 5.

The reaction products were analyzed on agarose gels, and bands between 5000 bp and 6000 bp were observed, matching the expected results at 5586 bp. Aliquots of successfully linearized plasmids were pooled together, and templates were cleaved with Fast Digest Dpnl at 37° C. for 30 min. Overhangs corresponding to the DE-loop and HI-loop were added to inserts via PCR with Phusion polymerase. As a template, a synthetic DNA construct containing all inserts to be cloned was used and primers were insert specific. Hence, the template was the same for all inserts and primers amplified the required insert. First, primer pairs for the amplification of rubredoxin and HiPIP were used to determine the concentration needed for the template to gain a successful PCR. The negative control contained MilliQ water in place of pET28a_Ena1B_no_his. Reaction products were analysed on a 0.8% agarose gel. All bands of overhangs with rubredoxin and HiPIP were found slightly under 250 bp and distinct from the negative control, which corresponded to the expected results of a successful PCR. The expected size of the constructs is given in the table below (Table 2). For the subsequent reactions a 10 times diluted template was used (0.22 ng/μL). PCR reaction products were analysed on a 0.8% agarose gel. All bands were found slightly under 250 bp and above 50 bp, which was calculated for successful PCR's (Table 2).

TABLE 2
Expected size (bp) of inserted constructs in the
DE-loop and HI-loop of Ena1B (SEQ ID NO: 8).

| Insert | Size with DE - overhang | Size in HI - overhang |
|---|---|---|
| Rubredoxin | 186 | 189 |
| HiPIP | 195 | 198 |
| WP_098221117.1 | 168 | 165 |
| FC702_01375 | 120 | 123 |
| WP_197262982.1 | 162 | 165 |
| WP_000861196 | 144 | 147 |
| WP_086405534.1 | 108 | 111 |
| WP_142338290.1 | 132 | 135 |

Inserts and vectors were ligated by homologous recombination through transformation in competent E. coli TOP10 cells. After transformation, cells were plated on LB-agar plates containing kanamycin. Colonies of different inserts were screened with colony PCR (cPCR) using vector-specific primers PETfw and PETrv. PCR products of selected clones were screened against the amplicon of pET28a_Ena1B_no_his at 600 bp using identical primers. Successful clones are expected to exhibit an upward shift of 111-198 bp on the agarose gel. PCR fragments or purified plasmids corresponding to selected clones were sent for Sanger sequencing (Eurofins) to identify successful constructs. The HI-loop insertion were all successful except for the insertion of FC702_01375. For the DE-loop, 4/8 (Rubredoxin, HiPIP, WP_197262982.1, and WP_142338290.1) were successful. Of the positive constructs, purified plasmids were transformed into the expression strain E. coli C43 (DE3) cells.

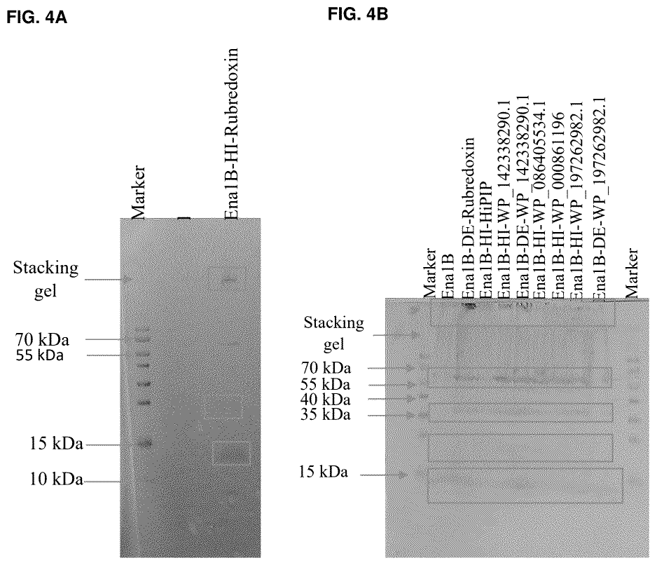

After cloning, constructs were expressed recombinantly in 200 ml of Lysogeny Broth (LB) with 50 ug/ml kanamycin. Overnight preculture were used to inoculate 200 mL LB using a 1:50 dilution factor, and expression was done by inducing at an OD₆₀₀ 0.9 with 1 mM IPTG for three hours at 30° C. After harvesting cell cultures, lysis was done by adding lysis buffer containing: 2% Dodecyl-β-maltoside (DDM), 0.1 mg/ml lysozyme, 10 mM ethylenediaminetetraacetic acid (EDTA), 500 mM NaCl and 50 mM Tris pH 7.5. This was left overnight at 37° C. Cell lysates were centrifuged at 35 000 g for 30 min to retrieve pellets. Afterwards, pellets were homogenized in MQ-water. To detect the presence of the Ena1B fusion proteins, SDS-PAGE, Western blot and negative stain imaging was done. As the Ena1B fiber is chemically and thermally resistant, we assume that formed Ena1B-metal binding protein (MBP) fusions will adhere to this characteristic. This means that, if fibers were formed, heat and chemical treatment will not depolymerize the Ena1B fiber or any of the Ena1B-MBP fusions, thus fiber will be stuck in the slots and in the stacking gel. SDS-PAGEs were run, and Western blots were done with the help of the iBind™ Western System. Primary antibodies rabbit anti-Ena1B, and secondary antibodies anti-rabbit alkaline phosphate were used to detect the presence of Ena1B-fusion proteins as shown in FIG. 4.

TABLE 3
Molecular weights of Ena1B-MBP fusion proteins.

| Ena1B-MBP fusion | Molecular weight (in kDa) |
|---|---|
| Ena1B | 12.03 |
| Ena1B-HiPIP | 17.8 |
| Ena1B-Rubredoxin | 17.4 |
| Ena1B-DE-WP_142338290.1 | 15.8 |
| Ena1B-HI-WP_086405534.1 | 14.7 |
| Ena1B-HI-WP_000861196 | 16.3 |
| Ena1B-DE-WP_197262982.1 | 16.6 |

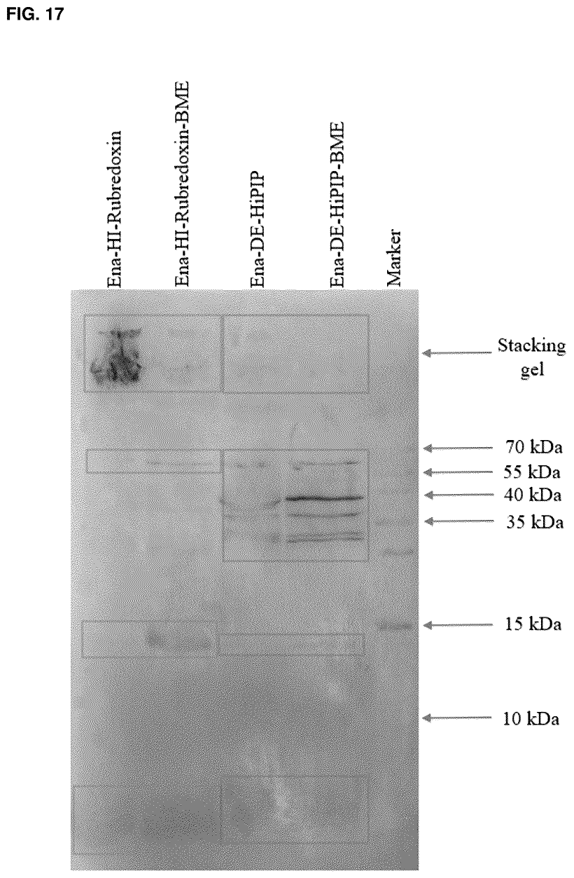

In the initial analysis, for the Ena1B-DE-HiPIP sample, no bands were visible, so these results for Ena1B-DE-HiPIP were inconclusive. A repeat of the same sample was performed however, as shown in FIG. 17, containing similar bands as Ena1B-HI-Rubredoxin, so the initial absence was a technical failure of the Western. For Ena-HI-Rubredoxin, bands of Ena1B were found in the stacking gel, between 55 kDa and 70 kDa, above 15 kDa and between 15 kDa and 10 kDa (FIG. 4a). In other constructs, an additional band was found within the loading slots of the stacking gel (FIG. 4b). Fibers formed are bigger than the pores of the polymerized gel, causing the fiber to get stuck between the stacking gel and the resolving gel, as well as within the slots of the stacking gel. Even though the intensities of detected Ena1B are dominant in the slots, a certain extent depolymerization still took place. As Ena1B and Ena1B-MBP fusions contain a molecular weight of 15-18 kDa (Table 3), we deduced that: tetramers were found between 55-70 kDa, trimers were found on thin bands slightly lower than 55 kDa, dimers are found on the band slightly above 35 kDa and monomers are on faint bands above 15 kDa. More bands are found under 15 kDa marker, likely indicating the degradation of monomers. Relative to their own lanes, degradation products are more present for the ENA-fusion constructs than for Ena1B, indicating that the Ena1B-MBP fusion constructs are different in structure and/or stability as compared to the Ena1B sample.

### Example 5. Fiber Formation of Ena1B-MBP Fusion Proteins

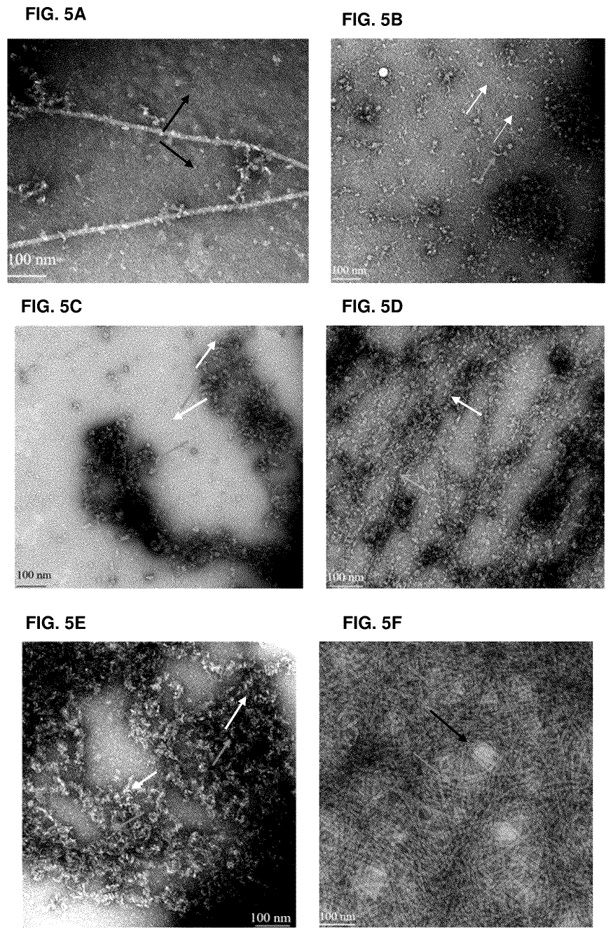

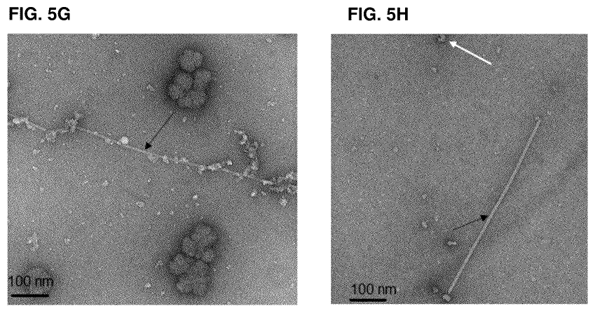

The presence of Ena1B in the wells does not determine if Ena1B-MBP fusions are present in fiber or aggregate form. For this reason, transmission electron microscopy is done to validate the presence of fibers. Negative stain TEM was the quickest way to determine if Ena1B-MBP fusion proteins formed fibers, which is why it was chosen for the viewing of the presence of Ena1B-MBP fusion proteins in fiber form. After lysis, Ena1B-metal binding protein (MBP) fusions were stored at −20° C. until ready for use. Aliquots of 5 μL were applied onto a copper mesh grid and analyzed with negative stain imaging. As previous western blots detected the presence of Ena1B in the slots of the gels, we hypothesized that on TEM images, though monomeric and low-level oligomeric proteins will be present, Ena1B-MBP fusion fibers will also be present. For Ena1B-DE-Rubredoxin (RR), fibers were seen (FIG. 5), albeit in a lower abundance than the Ena1B fibers without insert (FIG. 5f). at first analysis, for the other DE-loop fusion constructs, no fibers were detected, however a significant amount of aggregates was found on the sample. Unidentified filaments in Ena1B-DE-WP_197262982.1 were detected (FIG. 5d). It is assumed that these are not Ena1B as they have a smaller diameter. By further analysis, we revealed that the intended substitution of the Proline in the DE-loop was with an arginine (R) residue instead of the initially intended Glycine (so TGTGR instead of TGTGG, and both are included in SEQ ID NO:126 wherein X is the substitution of Pro with Gly or Arg), which may explain why fiber formation was less efficient or very difficult as the arginine will more likely interfere during self-assembly. However, the Ena1B-DE-RR, this linker insertion still allowed fiber formation in the first analysis.

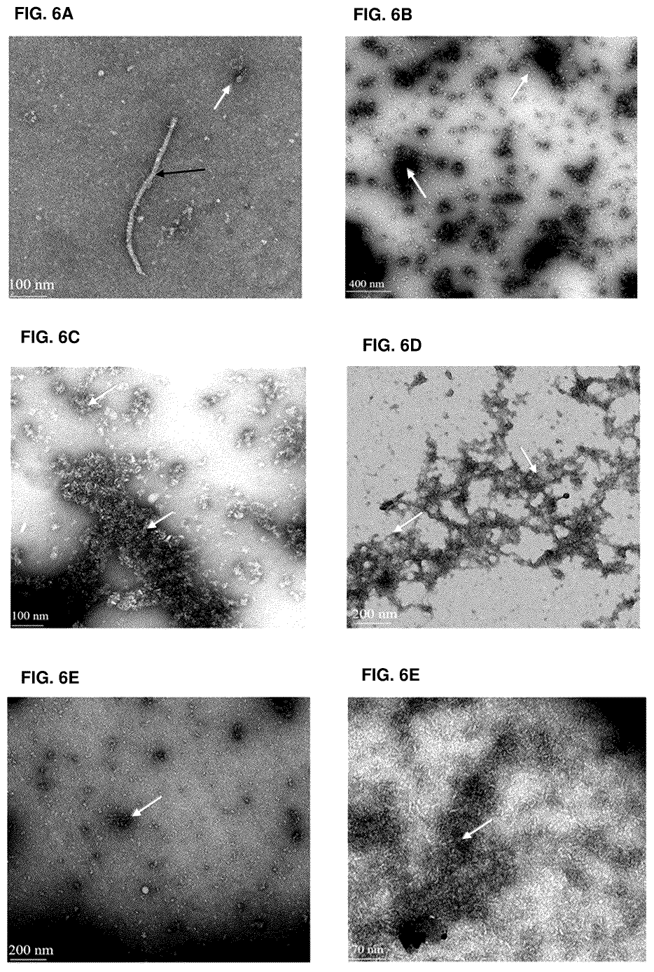

For the HI-loop fusion proteins, all constructs but Ena1B-HI-rubredoxin showed the presence of aggregates instead of fibers (FIG. 6). Again, the abundance was lower than for WT Ena1B fibers, but more abundant than for Ena1B-DE-Rubredoxin (which has the R at the Proline position of Ena1B in the fusion).

It is now evident that the detected Ena1B-MBP fusions in the wells of the previous western blots were aggregates that did not depolymerize. By looking at Ena1B-HI-WP_142338290.1 (FIG. 6f), it is assumed that certain aggregates were made of monomers and fibers that halted self-assembly after a few monomers, which were hereafter called spirals as a kind of multimeric assemblies. We hypothesize that among others, the forming of aggregates may be caused by incorrect docking of (mis) folded proteins to result in spirals. This misfolding may have been caused on the one hand by steric clashes within the fiber, causing it to misfold during self-assembly then aggregate, or aggregates are the result of protein that were produced at a rate that they were unable to fold properly. Indeed, literature stated that inclusion body formation as well as proteolytic degradation is frequent during high levels of heterologous overexpression in E. coli (Singh et al., 2015; Bhatwa et al., 2021), this includes insertional fusions (Chung Sei Kim et al., 2009). As level of expression exceeds 2% of total cellular proteins, the forming of inclusion body proceeds (Singh et al., 2015). To combat this problem, different approaches may be taken. Proper folding can be promoted by reducing the synthesis rate to reduce the metabolic burden (Bhatwa et al., 2021; Singh et al., 2015). Solutions include replacing the promoter for a weaker one and/or decreasing the inducing concentration (Singh et al., 2015; Bhatwa et al., 2021). A reduced temperature during induction slows the rate of transcription and translation as well as reduces the strength of hydrophobic interactions (de Groot and Ventura, 2006; Bhatwa et al., 2021; Singh et al., 2015). Cold-inducible promoters such as cspA can also be used (Qing et al., 2004). Cytoplasmic chaperones can be co-expressed on the plasmids or added in vitro to aid in folding of the fibers (DnaK-DnaJ, GroEL-GroES) (Chung Sei Kim et al., 2009).

For Ena1B-MBP fusions that formed fibers in this initial experiments (Ena1B-DE-Rubredoxin and Ena1B-HI-Rubredoxin), successful formation of fibers proved that aggregation in all constructs was possibly due to heterologous overexpression, requiring thus further optimization for their recombinant expression (see below Example 14).

For Ena1B-DE-Rubredoxin and Ena-HI-Rubredoxin, expression and purification were optimized to gain a higher yield. The OD₆₀₀ at induction was increased to 1.2 and expression was continued for 18 h at 20° C. Next, cells were lysed overnight in 1×PBS, 1 mg/ml lysozyme, DNAse 0.15 mg/mL, 10 mM EDTA and 1% (w/v) DDM then incubated at 99° C. in 1% (w/v) SDS for 15 min. From a two-liter LB culture, 5.77 g protein was isolated after cell lysis and SDS extraction.

### Example 6. Structural Analysis of Ena1B-Rubredoxin Fibers

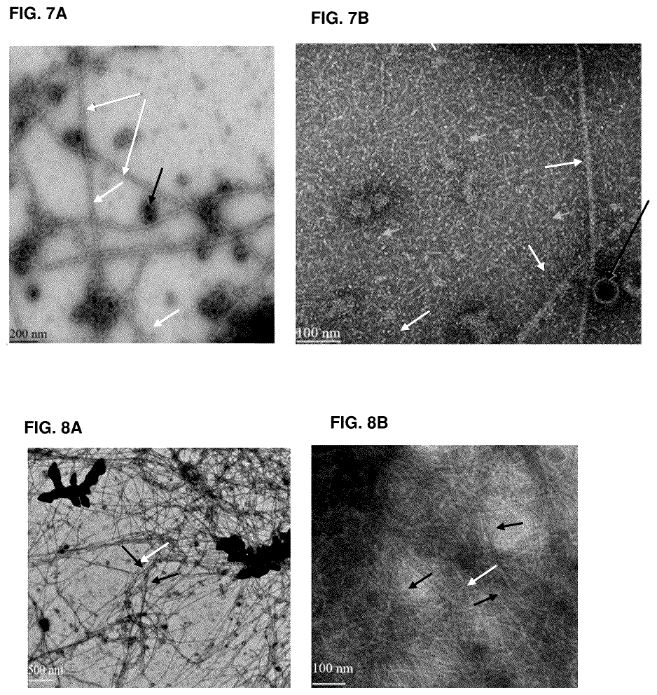

For purified Ena1B-DE-Rubredoxin, more fibers were found in the novel TEM images compared to the first recombinant production (FIGS. 5a and 7). Small spirals were also found in the sample (FIG. 7b) as found in Ena1B-HI-WP_142338290.1. Looking back at the sequence of the insertion of the rubredoxin loop into the Ena1B DE-loop, it is possible that longer linkers should be used to create more energetically favorable insertions into the loop. On the other hand, the glycine residue which was intended to be added as a linker (see table 1) resulted in insertion of an arginine (cloning artefact), possibly causing a lower efficiency in fiber formation. As to reduce the chances of not obtaining fibers, and to relieve steric hindrances caused by the linker amino acid identity, optimizing the constructs is thus possible by the tunability of the linker sequences.

Purified Ena1B-HI-Rubredoxin had a significantly higher yield than Ena1B-DE-Rubredoxin (with the Arg linker), as well as its previous negative stain visualization in the previous section (FIGS. 8 and 6a).

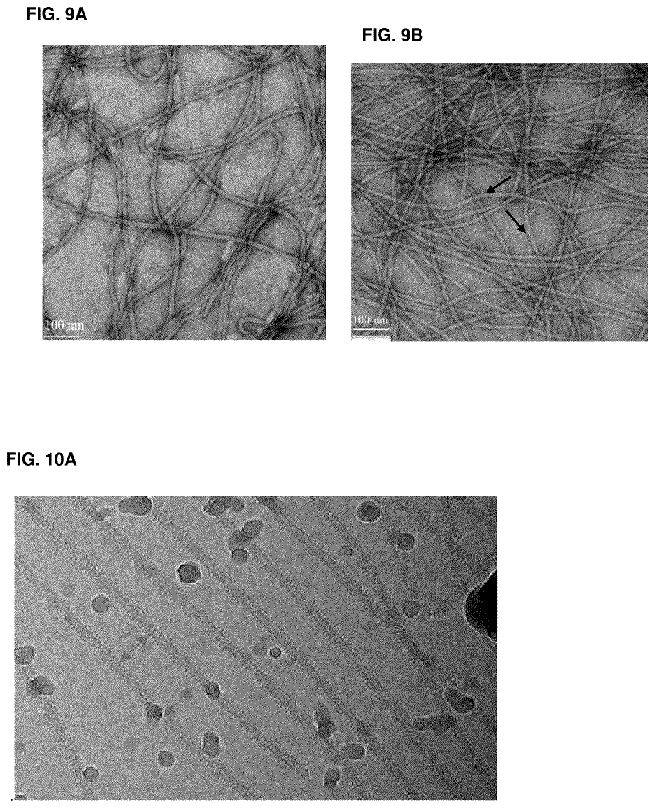

Samples were further purified by incubating the samples at 99° C. for 30 min, centrifuging at 30.130 g for 30 min then washing with 500 μL MQ-water three times. A pre-screening of Ena1B-DE-Rubredoxin via negative stain TEM imaging revealed that fibers were washed out during this stage. The pre-screening of Ena1B-HI-Rubredoxin via negative stain imaging determined that fibers did not contain any contaminants (FIG. 9a). The image also indicated that fibers of Ena1B-HI-Rubredoxin possess more serrated edges in comparison to the WT Ena1B fibers (FIG. 9b), suggesting the display of rubredoxin on the Ena1B fiber. An enquiry regarding the state of the RR-domain (folded/unfolded). As no contaminants were found, the sample was retained for cryo-EM analysis, which would help explain the rough edges found on the fibers.

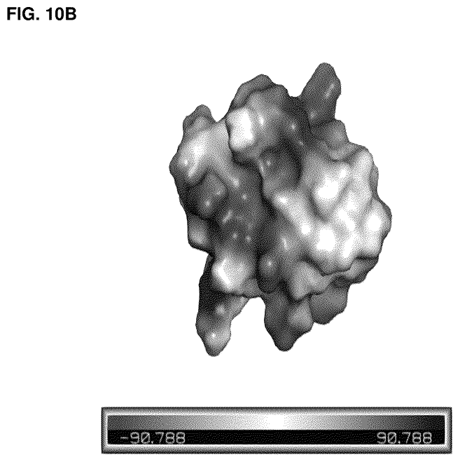

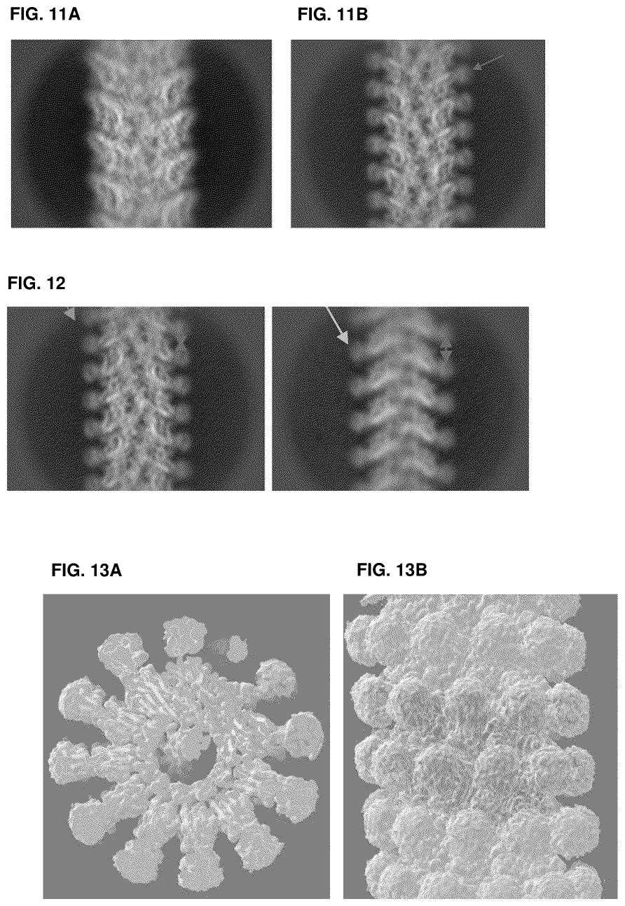

Next, purified Ena1B-HI-Rubredoxin fibers were prepared for cryogenic electron microscopy. 2D micrograph movies were recorded with the cryoEM with settings detailed in materials and methods, and the structure was solved with help of CryoSPARC™ and Wincoot (see below). While cryogenic plunging was done, parallel Ena1B-HI-Rubredoxin fibers were oriented at consistent distances on the grid (FIG. 10). A hypothesis for the arrangements stems from rubredoxins forming a repulsive electrostatic interaction between neighboring fibers, creating a nearly constant distance between them. This hypothesis was supported by the electrostatic map of rubredoxin, which proved that rubredoxin showed a repulsive force distally from the fiber. CryoEM data was processed in CryoSPARC™. Movies were imported into CryoSPARC™ and motion corrected. Contrast Transfer Function (CTF)-estimation estimated the resolution and defocus of each image. Hereafter, 5 Å cut-off was introduced to the micrographs. Through template-free filament picking, boxes of dimensions 380 Å×380 Å with distance of 5.6 nm were extracted along the length of straight Ena1B-HI-Rubredoxin filaments. 2D-classifications were done on twofold binned particles to filter out particles with a resolution upward of 5 Å. Preliminary results show that Ena1B-HI-Rubredoxin is in the form of a fiber which is similar to the folded Ena1B fiber (Pradhan et al., 2021) (FIG. 10). 2D class averages displayed a helical symmetry of Ena1B-HI-Rubredoxin (FIG. 10b and FIG. 11). The helical symmetry of Ena1B-HI-Rubredoxin to Ena1B is similar. However, fibers possess extremities in vague densities. An explanation of this is that as 2D-classes were stacked, focus was placed on the orientation of the helix and not rubredoxin. These vague densities are shown in the form of a ball similar to the form of a folded rubredoxin, though not yet conclusive (FIG. 2a). The extremities, or rubredoxins, are dynamic. Rubredoxin, while still grafted to the monomer, can move in 3 dimensions relative to the fiber. We assume that because of the vague densities, the 3D-reconstruction will contain volumes and density maps where the local resolution of rubredoxin is lower than the fiber. 2D class averages of Ena1B-HI-rubredoxin also displayed diameters of 142.7 Å and 134.7 Å (FIG. 12). As 2D-class averages of the smaller diameter are in the minority, it could be hypothesized that the Ena1B-HI-Rubredoxin fibers display a longitudinal stretching, which can be displayed by the difference in vertical distances between the rubredoxins combined with the difference in diameter of the fiber. The majority class and minority class have a longitudinal distance of 16.7 Å and 20.2 Å, respectively, between two rubredoxins (FIG. 12). As Ena1B fibers can also be formed without the docking of the N terminal connector, a second hypothesis it that Ntcs were not docked in the preceding subunits in the minority class Ntc's.

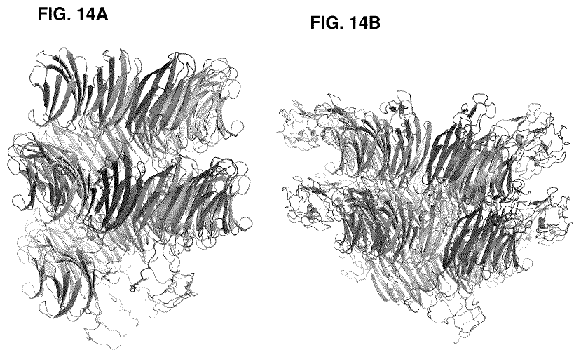

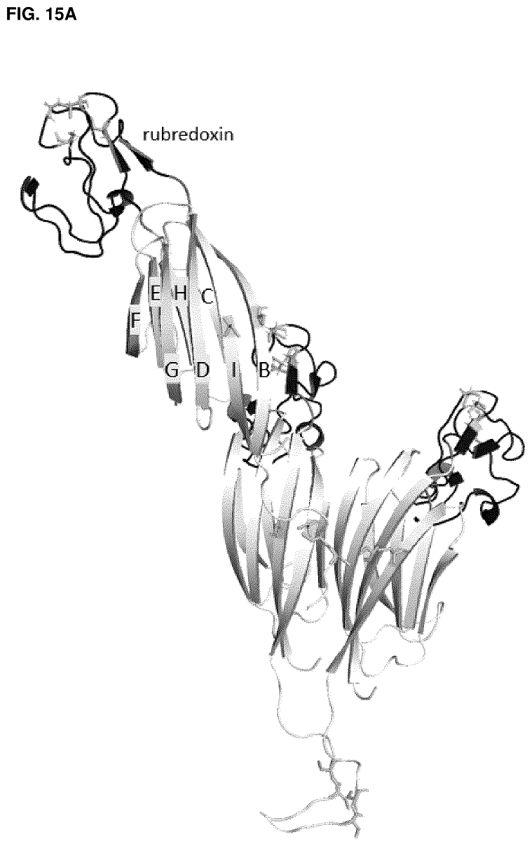

After filtering out low resolution (<5 Å) 2D-class averages, a helical refinement applied using initial rise and twist values from the Ena1B fiber, and a cylinder of 110 Å. A 3.11 Å preliminary 3D reconstructed volume of Ena-HI-Rubredoxin was formed. Recentered particles from the helical refinement were re-extracted and a second helical refinement was initiated using the preliminary 3D reconstructed volume. A final volume at 2.30 Å resolution was reconstructed with a refined helical rise and twist of 3.15 Å and 31.1 degrees. Model building was done on the basis of the pdb files of rubredoxin (2KKD) and Ena1B (7A02) to create an Ena1B-HI-Rubredoxin fiber. First, monomeric rubredoxin and Ena1B were docked into a density modified map of the volume. Connecting loops were built manually and the whole monomer was fitted into the map. This was then multiplied to gain an Ena1B-HI-Rubredoxin fiber (FIGS. 13 and 14). After solving the architectural structure, we can prove that a folded Ena1B-HI-Rubredoxin exists (FIG. 14). It possesses a width of 142.7 Å, a rise of 3.15 Å and a twist of 31.1 degrees. This differs to the Ena1B fiber by a difference of 32.7 Å, 0.287 Å and 1.25 degrees respectively (Pradhan et al., 2021) (FIG. 14). Thus, Ena1B-HI-Rubredoxin has the same staggered appearance as the Ena1B fiber (FIG. 14). We also prove that fibers of Ena1B-HI-Rubredoxin are near isomorphous to Ena1B. The folded Ena1B-HI-Rubredoxin accepts the native subunit-subunit B-augmentation contacts adopted by S-ENA as well as Ena1B fibers, which is described in the next section (Pradhan et al., 2021).

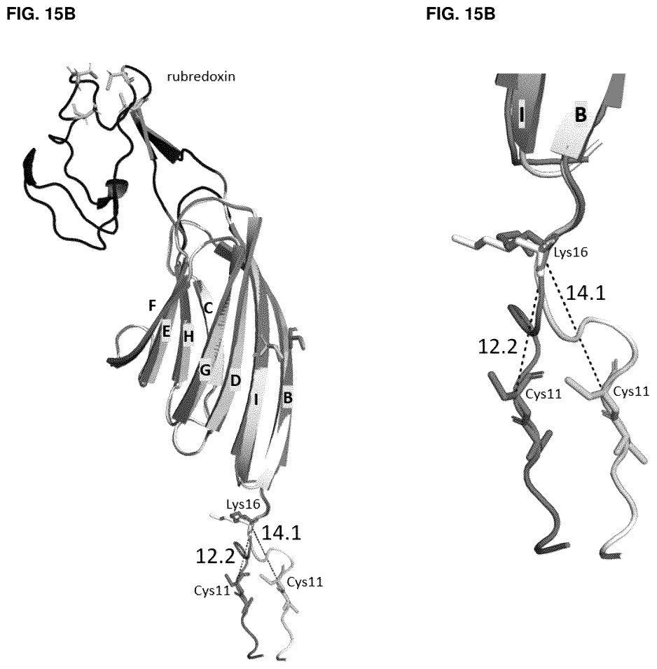

A monomer of Ena1B-HI-Rubredoxin contains a similar jellyroll fold domain, and N-terminal connector (Ntc), as Ena1B (FIG. 15). While Ena1B subunit maintains complementary electrostatic patches between subunits, the negative electrostatic charge on the rubredoxin repulse each other. This causes not only axial-distal, but also lateral electrostatic repulsions between the subunits. Ntc's form covalent bonds with cysteines in the 9^{th} and 10^{th} preceding subunits as done in Ena1B fibers (FIG. 15). The Ntc displays its flexibility by extending to form disulfide bonds with the I and B strands of the 9^{th} and 10^{th} preceding positions (FIG. 15 b,c) (Pradhan et al., 2021). This can be noticed when comparing measurements of the docking site as well as the beginning of the Ena1B domain on both Ena-HI-Rubredoxin and Ena1B (FIG. 15 b,c). The distance between Cys11 and Lys16 in Ena1B and Ena1B-HI-Rubredoxin amount to 12.2 Å and 14.11 Å. Though Ena1B-HI-Rubredoxin has electrostatic repulsions, lateral contacts between the subunits are still near isomorphous to Ena1B fiber. Subunits in the Ena1B fiber are also connected via helical turns as in Ena1B fiber (Pradhan et al., 2021).

### Example 7. Detection of Metals in Ena1B-MBP Fusions

To demonstrate the functionality of the metal binding proteins as inserted in the Ena1B protein, a metal detection assay was done to qualitatively measure the occupancy of metals docked into the centers of the MBPs. X-ray fluorescence displayed the (lack of) presence of metals in our folded rubredoxin. If metals are docked in the MBP, X-ray fluorescence also reveals the nature of the bound metal as well as the approximate ratio. With the help of X-ray fluorescence, peaks at different energy levels were measured to determine the presence of certain metals (FIG. 16). Residual metals were removed by incubating proteins at 100° C. in 1% (w/v) SDS for 30 min and subsequently centrifuged at 30 130 g for 30 min. Pellets were washed and centrifuged again at 30 130 g for 30 min to remove SDS and possible last residual metals. Proteins were fished with a loop and flash frozen in liquid nitrogen. Samples were sent for X-ray fluorescence analysis. As concentrations of the samples are unknown, peaks were only viewed in a relative manner (FIG. 16). Except for the negative control (Ena1B with HA tag inserted), distinct peaks were found in all samples, indicating the presence of metals in our samples. The detection of absorbance in visible wavelengths of the pellets and the presence of metals in metal-binding regions makes one hypothesize that proteins were indeed folded (Hough, 2017), either in aggregate form or fibers. Metals are bound by the metal center, which causes changes in absorbance in the visible spectrum according to the redox state of the metal (FIG. 16).

Ena1B-DE-WP_142338290.1, a gray ribbon zinc-binding protein, contained high levels of copper and a low level of iron (FIG. 16). This contradicts the expected results, as a BLASTp against the database yielded a zinc-binding protein (FIG. 1). As the protein yielded aggregates in first instance, no further investigation was done. The pale yellow Ena1B-DE-HiPIP contained levels of iron which corresponded to literature regarding the iron-sulfur protein (Carter, 2006; Messerschmidt et al., 2006; Bruscella et al., 2005) (FIG. 16). Nevertheless, Ena1B-DE-HiPIP produced aggregates in first instance causing a halt in experiments. In the red Ena1B-HI-Rubredoxin, high levels of zinc and lower levels of iron were detected (FIG. 16). This was indeed in correspondence with the literature, which states that overproduction in E. coli in Lysogeny Broth (LB) medium results in an abundance of zinc-binding rubredoxins (Meyer and Moulis, 2006; Mathieu et al., 1992; Bonomi et al., 1998). To avoid this, growth of Ena1B-HI-Rubredoxin in E. coli is yet to be done in minimal medium with an excess of iron. This has also already been done to express HET-Rd (Altamura et al., 2017), as well as rubredoxins without carrier proteins (Meyer and Moulis, 2006; May and Kuo, 1977). It is known that substitution of metals in rubredoxins is done by adding excess of desired metals in a reduced environment (Meyer and Moulis, 2006; Moura et al., 1991). Therefore, nickel-exchange was tested by reducing Ena1B-HI-Rubredoxin with an excess of sodium dithionite (10 mM), then washed to gain apo-proteins. Hereafter, Ena1B-HI-Rubredoxin was incubated with 10 mM NiCl₂ for 15 min. After centrifugation for 30 min at 30 130 g, the pellet had a yellow color. The yellow color could correspond with a successfully exchange to nickel in rubredoxins which is also stated in (Moura et al., 1991). The nickel-exchange in Ena1B-HI-Rubredoxin revealed that nickel and copper partially substituted zinc and iron (FIG. 16), indicating a partially successful metal exchange. A successful exchange indicated that through incubation with sodium dithionite, apo-Ena1B-HI-Rubredoxins were obtained, which then captured metals from its environment. This could be applicable in bioremediation, where the capture of heavy metals in wastewater can be advantageous. The capture of cadmium, mercury, gallium has already been done by rubredoxin synthesized by Clostridium pasterurianum (Maher et al., 2004). An exchange with nickel could also be promising for the forming of hydrogen gas. As mentioned in (Slater et al., 2018), nickel bound by rubredoxin forms H₂ through a mechanism similar to NiFe hydrogenases. Slater et al. suggest that a Ni¹ center with an adjacent protonated residue reaches a composition of Ni^{III}-hydride, which leads to H₂ through proton-coupled reduction. With the combination of Ena1B-HI-Rubredoxin as a carrier protein, industrial applications for the forming of hydrogen gas are promising.

### Example 8. Stability of Ena1B-MBP Fusion Protein Fibers

To determine if recombinant fiber Ena1B-HI-Rubredoxin is a stable component for the construction of a nanowire, stabilities of Ena1B-HI-Rubredoxin were measured through exposure in a reducing environment and heat. Analyses of the resulting products were performed by western blot to detect traces of degradation of Ena1B-HI-Rubredoxin as a fiber in a heated and reduced environment, as well as spectrophotometric analyses to measure degree of degradation of the rubredoxin in Ena1B-HI-Rubredoxin at high temperatures.

Ena fibers are known to be resistant to reducing agents such as even when strong reducing agents such as beta-mercaptoethanol (BME) (Pradhan et al., 2021; Zegeye et al., 2021). The resistance of Ena1B-HI-Rubredoxin to BME was analyzed and Ena1B was used as positive control while Ena1B-DE-HiPIP, an aggregate which would thus definitely be degraded or reduced by BME as a negative control. A Western blot was done to compare the stability between Ena1B, Ena1B-HI-Rubredoxin and Ena1B-DE-HiPIP in its oxidized and reduced form. After cell lysis, pelleted lysates were incubated in 40 μL in 1% (w/v) SDS with and without 1 μL 2.5% v/v BME at 100° C. for 15 min. 30 μL of LDS-heat inactivated samples were loaded on SDS-PAGE, and Western blot was performed with the iBind™ Western System, using primary antibodies rabbit anti-Ena1B, and secondary antibodies anti-rabbit alkaline phosphate detected the presence of Ena1B-fusion proteins as shown in FIG. 17. Ena1B-DE-HiPIP, displays smaller degraded oligomers when the reducing agent is added, declaring that the aggregates of Ena1B-DE-HiPIP are indeed not as chemically stable as Ena1B fibers. Aggregates are hereby susceptible to BME-reducing degradation. Similar to Ena1B, no significant difference is observed between the presence or absence of reducing agent in the lanes of Ena1B-HI-Rubredoxin, suggesting a stable and robust fusion is formed, resistant to reducing agents.

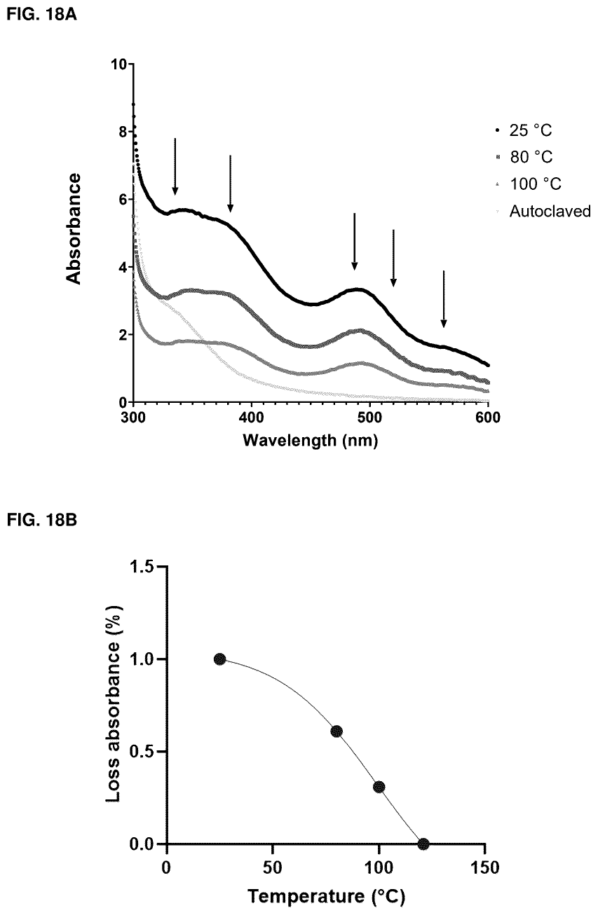

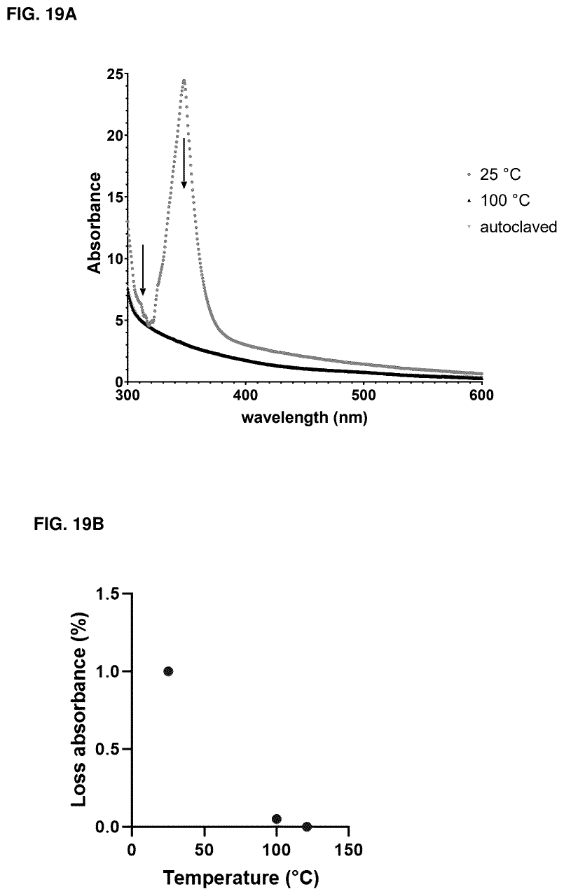

Next, the melting temperature (Miotto et al., 2019.) was determined, by incubating 50 ul of 1.23 mM purified Ena-HI-Rubredoxin in 80° C., 100° C. and 121° C. (autoclave) for 25 min. FIG. 18a shows the UV-Vis spectra measured from 250 nm to 700 nm. A typical UV-Vis spectrum of rubredoxin of Desulfovibrio vulgaris peaks at 380 nm, 490 nm, and 570 nm (E. D. Coulter and Kurtz, 2001), which we also see as a blue line in FIG. 18a. A lack of shift in the typical spectrum of rubredoxin in its free form as well as fused to a carrier protein, shows that the iron center of rubredoxin remains unchanged. Accordingly, the function of a rubredoxin remains unchanged whether bound or unbound by Ena1B. A gradual loss of the typical spectrum of rubredoxin (E. D. Coulter and Kurtz, 2001) was observed with the increase of temperature. We assume that this loss was caused by a gradual degradation of rubredoxin. A thermal denaturation curve nearly identical to FIG. 18 can be seen in (Cavagnero et al., 1995), confirming our hypothesis. A complete loss of the typical UV-Vis spectrum only happened when Ena1B-HI-Rubredoxin was autoclaved (purple, FIG. 19), indicating that rubredoxin on the Ena1B fiber completely unfolded (Cavagnero et al., 1995; Bonomi et al., 2000; Botelho and Gomes, 2011) at 121° C. and 1.2 bar. This remained the same after 72 hours (data not shown), indicating that the unfolding was irreversible under the buffer conditions tried. This corresponds to the literature where it states that thermal denaturation of rubredoxins is irreversible (Cavagnero et al., 1998; Bonomi et al., 2000; Marly K. Eidsness et al., 1997; Cavagnero et al., 1998). So, the melting temperature, or the temperature at which the concentration of oxidized Ena1B-HI-Rubredoxin is at 50%, is estimated to be 98° C., indicating its high stability.

Melting temperatures of reduced rubredoxin in the HI-loop of Ena1B fibers were also calculated by incubating 1.21 mM of Ena1B-HI-Rubredoxin in 100° C. and autoclaving for 25 min under anaerobic conditions. The graph obtained is as shown in FIG. 19. No gradual loss of reduced iron was observed here. Rather, the trends of the autoclaved and incubated at 100° C. revealed that the loss of the iron atom in Ena1B-HI-Rubredoxin happened at a much lower temperature (Bonomi et al., 2000). Ena1B-HI-Rubredoxin in a heated, reduced environment, forms apo-proteins. This makes the protein non-functional, regardless of the folding state of the protein. Literature already stated that oxidized rubredoxin is more thermally stable than its reduced form (Meyer and Moulis, 2006; Bonomi et al., 2000). Current observation lead us to the understanding that this also applies to rubredoxins bound to Ena1B-HI-Rubredoxin.

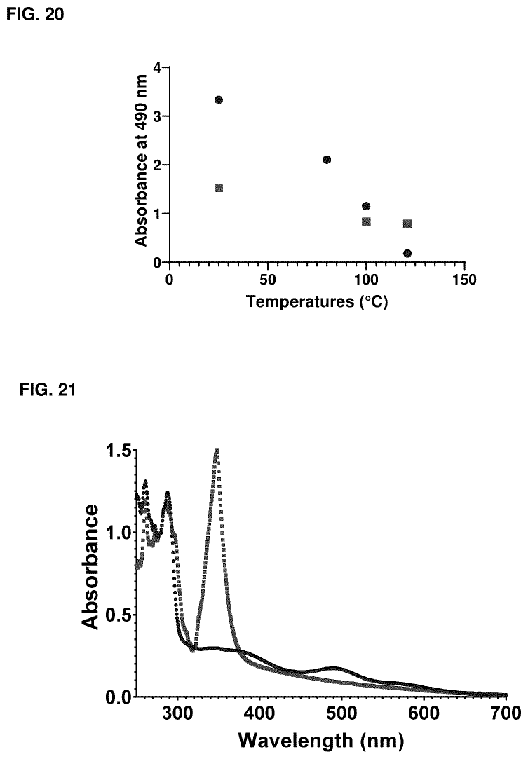

Finally, the stability of Ena1B-HI-Rubredoxin was also analyzed as a measure of the half-life of Rubredoxin, as present in the HI-loop of Ena1B, by incubating 50 μL of Ena1B-HI-Rubredoxin at 80° C. and measuring the UV-Vis spectrum every ten minutes. The denaturation of the protein is determined by the loss of the absorption at wavelengths 320-380 nm, 490 nm, and 570 nm as shown in FIG. 20. By heating the protein, the denaturation starts and the iron center gets disturbed, which bleached the typical absorption spectrum of oxidized rubredoxin. Similar curves were also found for Clostridium pasteurianum rubredoxin and Pyrococcus furiosus rubredoxin in (Marly K Eidsness et al., 1997). In oxidized rubredoxin, the declining trend is viewed as a mono exponential curve (Pais et al., 2005), which brings the half-life at 80° C. to 170.38±22 min. As no half-life is measured for rubredoxin derived from Desulfovibrio vulgaris, comparison is done with Desulfovibrio gigas. In a study, recombinant rubredoxin from Desulfovibrio gigas has a half-life of 167 minutes at 80° C. (Papavassiliou and Hatchikian, 1985). In another study, the half-life at 90° C. is measured at 96 minutes (Pais et al., 2005). Its stability therefore remains in the range with other mesophilic rubredoxins. Other rubredoxins isolated from the thermophile Thermodesulfobacterium commune and hyperthermophile Pyrococcus furiosus have a half-life of 6 h (Papavassiliou and Hatchikian, 1985) and 24 h (Christen et al., 1997) at 80° C. which are much more thermally stable proteins.

As nanowires, thermal stability is needed to tolerate high temperatures during utilization. Ena1B-HI-Rubredoxin is able to endure thermal exposure for an amount of time equivalent to other mesophilic rubredoxins (unbound to Ena1B), however half-lives can always be improved by grafting rubredoxins isolated from thermophiles and hyperthermophiles.

### Example 9. Redox Activity of Ena1B-HI-Rubredoxin

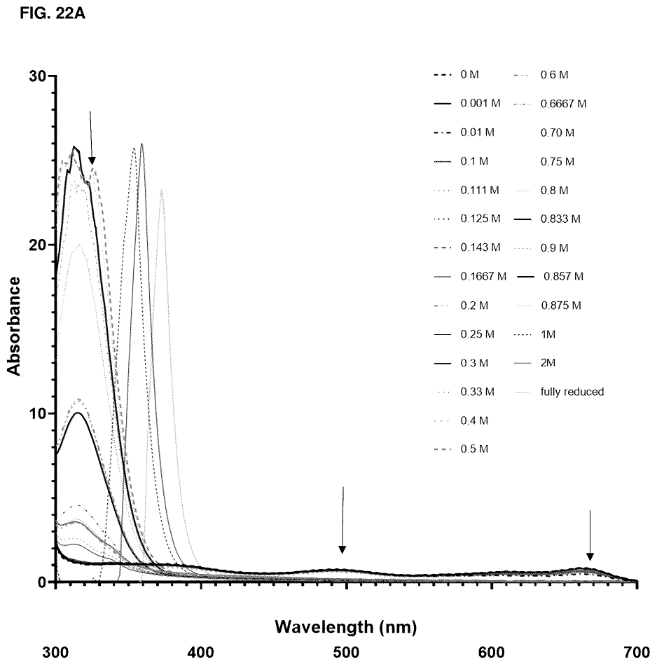

As to investigate the suitability of the Ena-MBP fusions, in particular the Ena1B-rubredoxin fusions, for use as metal-binding nanowires with redox activity, analysis was performed herein to get an indication on their redox potential. The more positive the redox potential, the more likely the reduction of the metal center happens (Hosseinzadeh and Lu, 2016). However, before measuring the redox potential, the type of reducing agents capable of reducing the metal site in Ena1B-HI-Rubredoxin had to be defined. As a starting point, the purified Ena1B-HI-Rubredoxin samples had a bright red colour, which, in line with the UV-VIS spectrum indicated the protein is predominantly in the oxidized state. Sodium dithionite, at redox potential-660 mV, reduces several organic groups such as aldehydes and ketones (Yan and Smith, 2001). A reduction was performed with sodium dithionite as reducing agent, which has a peak in absorbance at 315 nm that gradually decreases with increasing concentration (FIG. 21). Sodium dithionite displays gradual shifts to 380 nm when an excess develops (data not shown). The progress of Ena1B-HI-Rubredoxin reduction was followed by monitoring the loss of absorption peaks in 320-380 nm, 490 nm, and 570 nm, which are replaced by the absorption shoulder at 310 nm and a large sodium dithionite peak at 340 nm (Meyer and Moulis, 2006), possibly overshadowing a second peak at 330 nm.

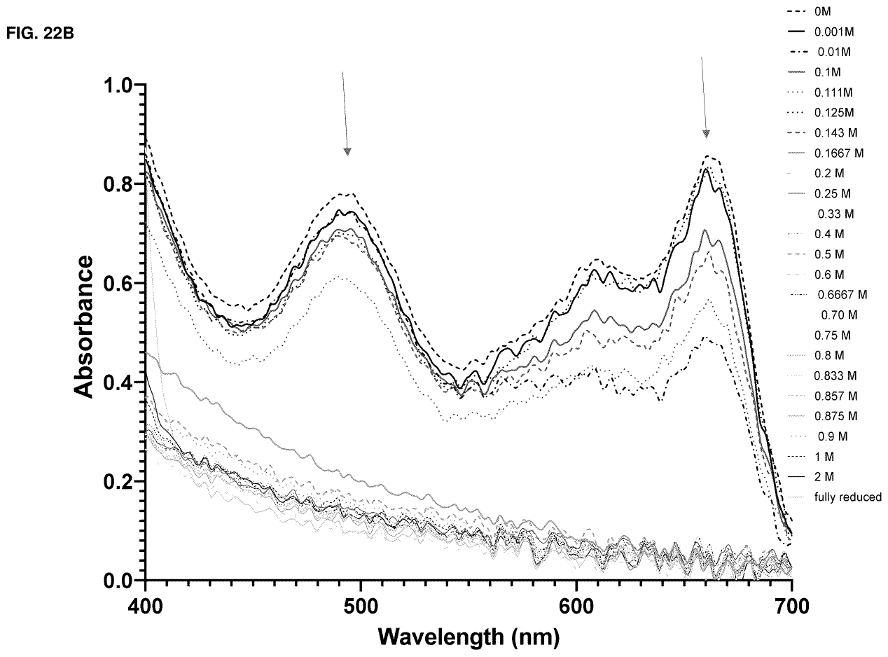

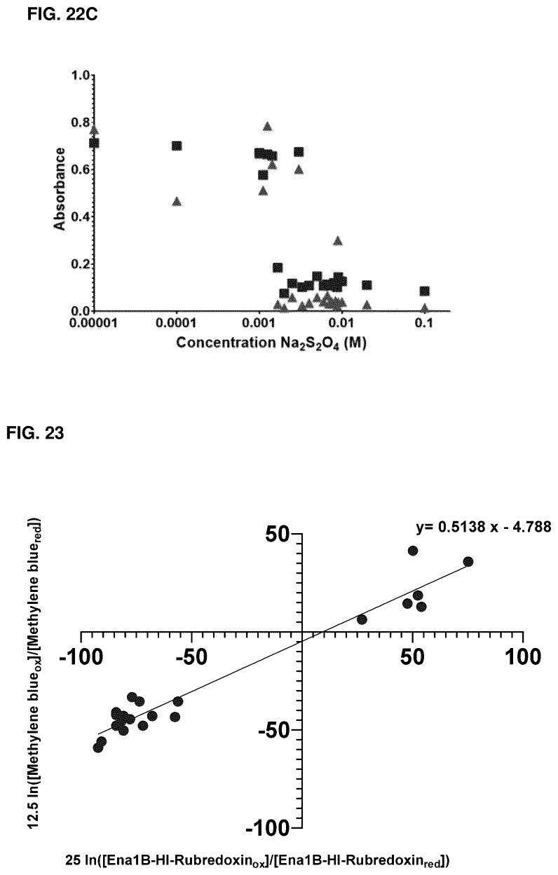

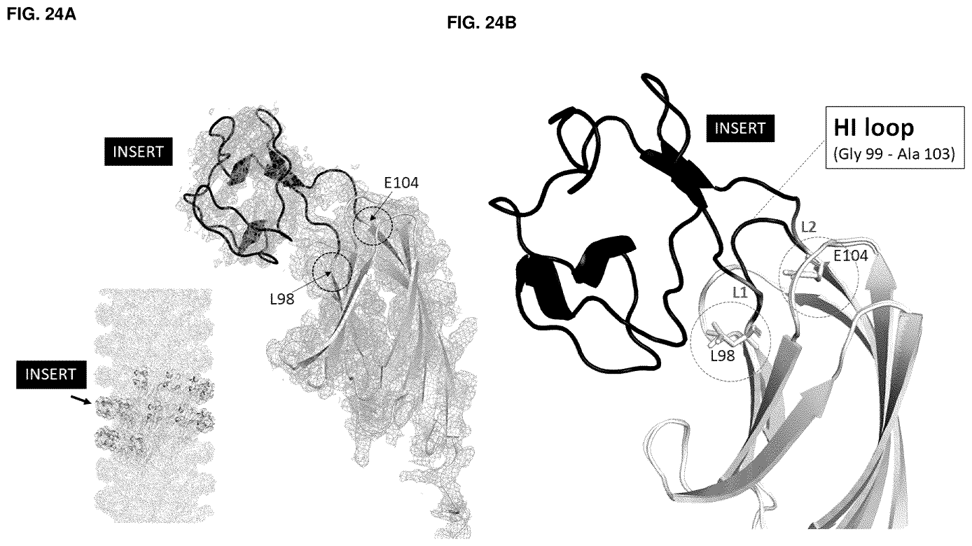

So next, a spectroscopic redox titration with the help of sodium dithionite as a reductant was performed to quantitively determine the redox activity of the fiber. Methylene blue (Eₘ=11 mV at pH 7) was added as redox indicator, with the concentration of the dye adjusted to have an absorbance at 668 nm approximately equal to 490 nm. The UV-Vis spectrum was measured with increasing concentrations of sodium dithionite (from 10⁻⁵ M to 10⁻¹ M). The increase of dithionite concentration can be monitored through as well as small peaks at 310 and 330 nm, indicating a reduction of the protein. The decrease at 480 nm and 668 nm was measured and displayed in a graph (FIG. 22). A Nernst Plot was also made based on the graphs, from which the redox potential was calculated (FIG. 23).

As rubredoxin's redox center remains unchanged after binding to the fiber, we assumed that the redox activity of rubredoxin would remain largely unchanged when bound to the Ena1B monomer. Ena1B-HI-Rubredoxin contains the rubredoxin from Desulfovibrio vulgaris strain Hildenborough with a redox potential of 0 mV (Fauque et al., 1987; Liu et al., 2014).

This assumption was proven correct, as the redox potential was calculated to be 6.2 mV+4.1 mV at pH 7 (though, the spectrophotometer fluctuations observed during measurements indicates a larger error, and these values have to be considered as an approximation until replications are performed).

Thus, functionality of rubredoxin on Ena1B-HI-Rubredoxin remains unchanged whether bound or unbound to the Ena1B-monomer. Ena1B-HI-Rubredoxin is also still highly redox active. This was proven by the spontaneity in which rubredoxin reoxidizes upon exposure to air.

### Example 10. Ena1B Modified with Biomineralizing Inserts

Metal binding proteins (MBPs) come in various functional classes. One of these functional classes are biomineralizing MBPs, able to bind and organize metal salts, for example calcium carbonates and phosphates. By binding and structurally organizing metal salts or oxides, biomineralizing MBPs can nucleate the crystallization, i.e. mineralization, of metal salts and oxides, and thus bias the mineralization process towards specific crystalline forms and locations (Dhami et al. 2013). Here, we engineered Ena1B fibers to incorporate and functionally display natural and engineered biomineralization (poly)peptides.

In this way, we obtain easy to produce and isolate self-assembling S-ENA fibers with a built in biomineralization property. Said fibers can be used to sequester metal salts from aqueous solutions, for example for the removal of calcium carbonate from hard waters. Said fibers can also be used as a fibrous scaffolding matrix for pure or composite materials based on biomineralization processes, for example in calcium carbonate, calcium phosphate and hydroxyapatite containing materials and minerals such as calcite cements, bone, enamels, nacres and shells.

Two examples where Ena1B is modified to incorporate and functionally display (1) natural and (2) engineered calcite biomineralization (poly) peptides are provided herein.

In a first example, the coding sequence of Sycon ciliatum diactinin (GenBank: SIP56239.1; SEQ ID: 157), which provides for a biomineralization protein of the calcareous sponges of the genus Sycon, is inserted into the HI loop region of Ena1B, with flanking linker sequences L1: GGG and L2: GGAA, thus resulting in a fusion protein hereafter referred to as Ena1B-HI-diactinin (SEQ ID: 158).

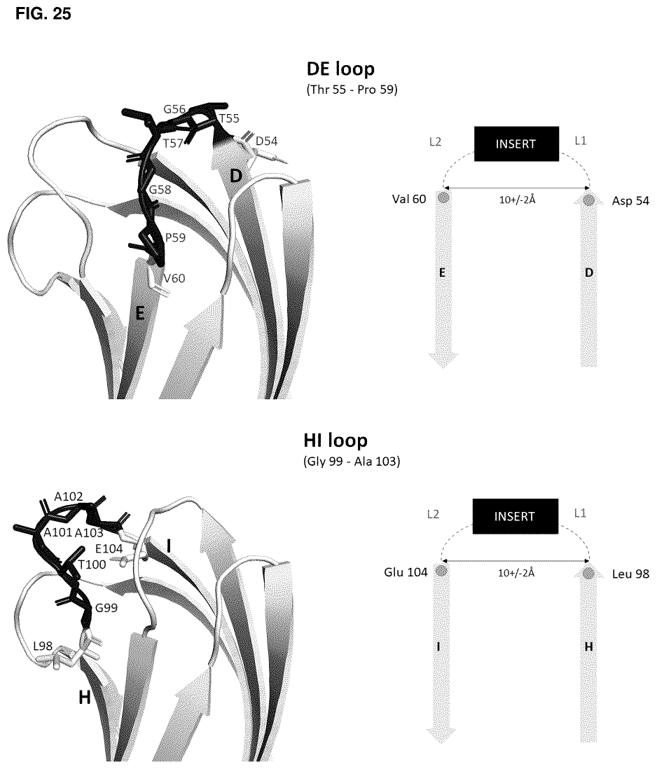

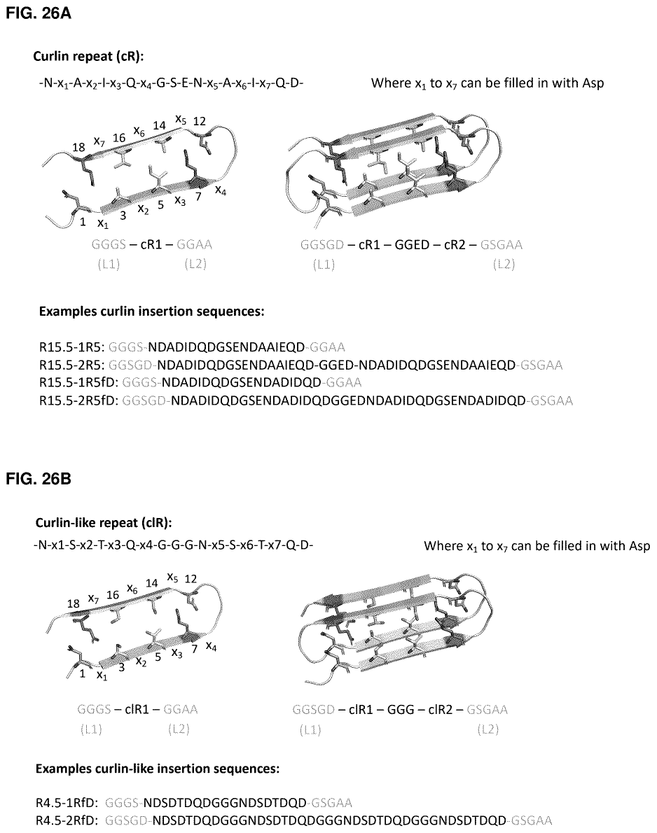

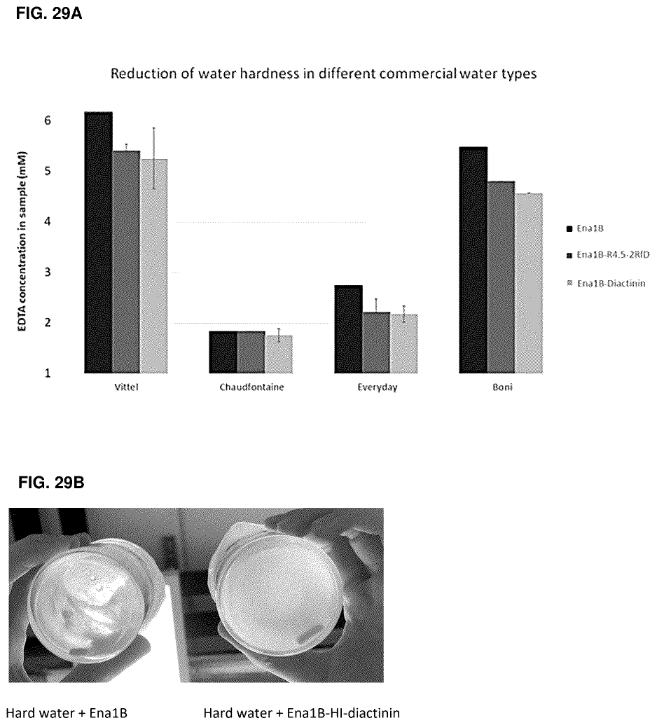

In the second example, we use curlin repeat and curlin-like repeat (referred to as cR and clR, respectively; FIG. 26) sequences as rigid, repetitive structural scaffolds to incorporate regularly spaced ladders of aspartic acid. We have previously shown curlin and curlin-like repeats to adopt a regular beta solenoid structure (Sleutel et al. 2022). Fusions with Ena1B can be made in the DE or HI loop, using a single or double repeat unit, separate by a sequence GGED for curlin repeats, or GGG for curli-like repeats (FIG. 26). These single or double repeat inserts are connected to the DE or HI loop anchor points of Ena1B (i.e. Asp54 and Val60 for DE, Leu98 and Glu 104 for HI) according to the insertion principle shown in FIG. 25. Per curlin repeat or curlin-like repeat, seven surface exposed residues (x1-x7; FIG. 26) can be substituted with any amino acid residue. When mutated to Asp, this generates repeats with one (x1, x2, x3 and x4) or both (x1 to x7) sides containing a regularly spaced ladder of aspartic acids. This structural organization of these aspartic acids results in calcium and calcium carbonate binding properties, and the ability to nucleate the mineralization of calcium carbonate (FIG. 28).

A non-limiting list of candidate calcium mineralizing curlin repeat and curlin-like repeat insertion sequences is provided in SEQ ID NOs: 159-164, or derivable from the motifs as represented by SEQ ID NOs: 178-179 (see FIG. 26), wherein the x is an Asp for obtaining calcium-binding motifs.

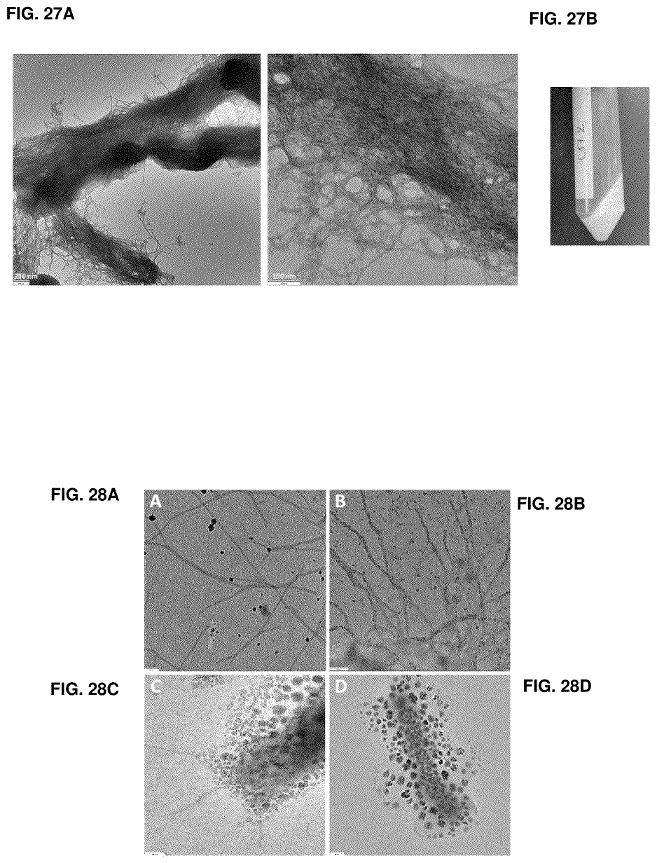

When expressed in E. coli the Ena1B-HI-diactinin and Ena1B-HI-R4.5-2RfD fusion protein gives rise to an abundance of S-ENA fibers (FIG. 27), encompassing as much as 40-50% of the total cell mass. Ena1B-HI-diactinin and Ena1B-HI-R4.5-2RfD fibers are harvested from the producing cells by cell lysis and a 2 hour incubation in a buffer containing lysozyme, allowing enzymatic digestion of the peptidoglycan cell wall. The resulting cell lysis is then incubated for one hour in 1% SDS at 100° C. to solubilize cellular proteins and membranes. Under these conditions Ena1B-fusion fibers stay intact and can be isolated by centrifugation of the Ena1B-fiber prep.

Ena1B-HI-diactinin and Ena1B-HI-R4.5-1RfD fusion proteins were then tested for their calcium binding and/or calcium mineralizing properties. To do so, fiber preps were added to various hard waters. Following addition of Ena1B-HI-diactinin and Ena1B-HI-R4.5-1RfD, but not wild type (Ena1B) fibers, a rapid sedimentation was seen from a precipitate corresponding to Ena1B-MBP fusion fibers and calcium carbonate salts. When analysed by TEM, these fiber pellets showed the presence of variable size salt crystals, corresponding to calcium ion salts. The fiber-calcium carbonate aggregates are easily removed from solution by sedimentation. To evaluate the water softening potential of the calcium carbonate mineralizing Ena1B-MBPfusion proteins, the concentration of residual divalent metal (i.e. predominantly Ca²⁺ and Mg²⁺) ions in the supernatants was analysed by an EDTA titration assay that follows free divalent ions by means of the indicator dye Eliochrome black. In this way, Ena1B-HI-diactinin and Ena1B-HI-R4.5-2RfD fusion proteins were found to rapidly remove an excess calcium from the hard waters (FIG. 29).

### Example 11. Towards a Generic Method for Constructing Ena Fusion Proteins

### Generation of Self-Assembling S-Ena Fusions by Polypeptide Insertion in Loops DE or HI.

A standardized non-limiting method to design and generate the Ena-insertion fusion proteins, in particular Ena-MBP fusions, as described herein, has been specifically outlined here for Ena1B-DE or Ena1B-HI insertions, and contains the following steps:
(1) Linearize the expression plasmid encoding Ena1B (Uniprot: A0A1Y6A695) wherein the DE loop (Thr55-Pro59), as provided in SEQ ID NO:137 encoding SEQ ID NO: 138, or HI loop (Gly99-Ala103), as provided in SEQ ID NO: 139 encoding SEQ ID NO: 140, has been removed, for example by outward PCR (FIG. 24);
(2) Select the coding sequence of the insert or target polypeptide to be inserted such that its N- and C-terminus are favorably positioned to connect to N- and C-termini of the DE- or HI-loop in the S-Ena scaffold, each spaced 10±2 Å (FIG. 25). Adjust the spacing of N- and C-terminus of folded insertion domains by selective removal of residues, ensuring that no residues are removed essential for folding of the insertion domain, or by addition of linker sequences. One can employ de novo structure prediction algorithms, the like of Alphafold2 or RoseTTAFold, and/or molecular dynamics simulation to validate the folding ability of the S-Ena fusion construct, and superimpose predicted structures of the S-Ena fusion onto the S-Ena fiber structure for Ena1B (SEQ ID NO:8), as available in PDB entry 7A02 to evaluate the presence of steric clashes in the fiber. Adjust linker sequences accordingly by rational design or empirically by linker shuffling;
(3) Insert the coding sequence encoding the target polypeptide, optionally including linker regions, by means of, in a non-limiting way, Gibson assembly, In-Fusion cloning, overlap PCR, PIPE PCR, ligation independent cloning (LIC) in the linearized expression plasmid at the DE- and/or HI-loop cleaved opening;
(4) Transform an expression host cell with the expression plasmid encoding the S-Ena fusion protein(s) designed in step (1-3). In a non-limiting way, a preferred expression host is E. coli, where expression of S-ENA fusions result in high yields (reaching 10-30% of cell mass) of self-assembled ENA fibers;
(5) Optionally, isolate or purify the recombinantly produced S-ENA fusion proteins and fibers, preferably by chemical or mechanical lysis of the host cells, with a facultative enzymatic digestion (by means of glycosyl hydrolases, nucleases or proteases) of host polymers such cell envelope polymers, DNA/RNA, and undesired protein polymers. Well-folded (active) recombinant S-Ena fibers are isolated and purified from contaminating proteins and cell debris by incubation in a heated (boiling) 1% solution of the denaturing detergent sodium dodecyl maltoside (SDS). S-ENA fibers retain integrity under said conditions and can be recovered by sedimentation.

In the examples described herein, a selection of folded or even intrinsically disordered metal binding domains or protein was used as an insert comprising metal binding properties to introduce redox-active centers, calcium and magnesium mineralization proteins, or metallothionines for (heavy) metal binding. However, equivalent procedures allow insertion of further protein inserts, particularly folded proteins including non-metal binding domains, thereby broadening the number of polypeptide sequences that can be used in the design for insertion and production as an Ena fusion protein.

Moreover, the generalized method as described herein above provides for the insertion in an S-Ena coding sequence, in particular Ena1B (as provided in SEQ ID NO:8 lacking DE- or HI-loop, as shown in SEQ ID NO: 137 and SEQ ID NO: 139, resp.), embedded in a circular plasmid. Alternative approaches to making coding sequences for S-Ena fusion proteins are possible, as known by the person skilled in the art. First, the alternative ENAs as for instance provided by SEQ ID NOs: 1-82, may be applied in a similar way. Second, one can also start for instance, from two linear Ena coding sequences, encoding the N-terminal region of S-Ena up until the connecting site residue (Asp54 for DE, Leu98 for HI loop insertions, resp.), and the C-terminal region of the S-Ena starting from the connecting site residue (Val60 for DE, or Glu104 for HI loop insertions, resp.). N- and C-terminal fragments of the S-Ena scaffold and the coding sequence of the insertion polypeptide can be fused in vitro or in vivo by different gene stitching methods known to the person skilled in the art.

### Method to Select the Insert or Target Polypeptide Domain to be Inserted

As described in the examples and above, the most important criterium for an insert to match in the S-Ena loop is that N- and C-termini are in proximity of each other, at a distance compatible with the distance created when opening the Ena loops (the latter being 10±2 Å).

This can be judged by the skilled person from analysis of the structure if available, or de novo structure prediction by algorithms such as AlphaFold2 and RoseTTAFold. If needed, amino acid residues can be deleted from the loop regions (truncated loops), and/or short linkers can be added to optimize the distance.

Several classes of metal-binding proteins have been exemplified herein and can further be used to provide clarification on the suitability of said polypeptides as insert for S- or L-Ena fusions. As a matter of example how the approach can be followed as outlined in this example for any of the proposed metal-binding protein classes, the insertion of the MBPs used in examples 1-3 is outlined below.

MBPs were selected or as known metal binding proteins for which a 3D-structure is provided in PDB (e.g. Rubredoxin, HiPIP), or proteins predicted as “putative metal binding proteins” (e.g. as performed in the examples based on all protein sequences of <200 aa from Bacillus thuringiensis kurstaki proteome, for which the structural prediction was obtained using AlphaFold2, followed by visual inspection of potential metal coordination by Cys and His residues-see Examples 2-3). For the MBPs exemplified herein, considering the predicted N-/C-terminus proximity and the required adaptation of the insert sequence for improving the suitability as an Ena1B insert was made as described in Examples 1-3 and Table 1.

The generic method as described here above in Example 11, wherein the DE- or HI-loop is absent in the expression plasmid however requires that the insert sequence is adapted in view of the SEQ ID NOs: 129-136 of Table 1, to further include those DE- or HI-loop residues, preferably substituted as described in Examples 1-3, and optionally including the flanking linker amino acid sequences where required. In view of the above protocol for inserting the polypeptides of interest, specifically the MBPs, in the Ena1B-A-DE/HI-loop expression plasmid sequence, as provided by SEQ ID NOs: 137 (DNA) and 138 (protein), or SEQ ID NOs: 139 (DNA) and 140 (protein), resp., the inserts of Table 1 have been adapted to the insert sequences comprising such loop and optional linker residues, to provide the inserts for step 3 of the generic method used above by any of SEQ ID NOs: 141-156.

A similar approach can thus be undertaken for the insertions and fusions in view of the calcium mineralization proteins, as outlined in Example 10, for which we started from known biomineralization proteins from different species; as well as for examples for inserting metallothionines for bioremediation (heavy metal binding).

### Example 12. Lactococcus Expression of S-Ena Fibers

The Ena1B gene was cloned in the pNZ8148 vector for nisin inducible expression in Lactococcus lactis. A liquid culture of a single colony was grown overnight at 30° C. in M17 medium, supplemented with 0.5% glucose and 12.5 ug/ml chloramphenicol. This overnight culture was used to inoculate 1/40 culture M17 medium, supplemented with 0.5% glucose and 12.5 ug/ml chloramphenicol and grown at 30° C. At an OD₆₀₀ nm of ±0.5 the protein expression was induced with 1 ng/ml nisin. Cells were harvested after 3 hours by centrifugation (4000 rcf, 15 min). The cell pellet was resuspended in 2 mg/ml lysozyme dissolved in 20 mM Tris pH 7.0, 50 mM NaCl, 5 mM EDTA and incubated overnight at 37 C. Subsequently, 2% SDS was added, and the sample was boiled for 30-60 minutes. S-Ena fibers were collected by centrifugation (30 minutes at 20.000rcf) and resuspended with miliQ prior to deposition on an electron microscopy grid and imaging by negative staining TEM (FIG. 34).

### Example 13. Production and Fiber Assembly of Ena1A-DE-Rubredoxin Fusion Proteins

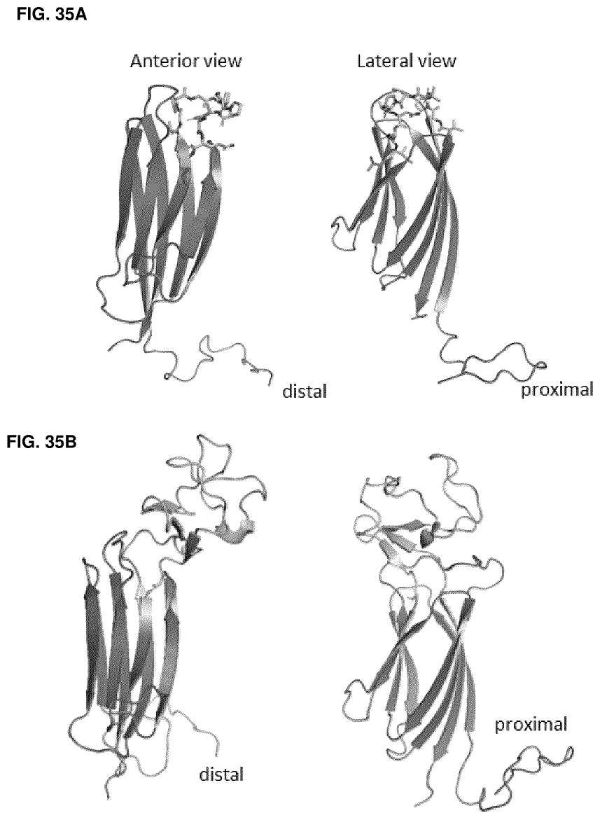

To further demonstrate the applicability of the design of S-Ena-MBP fusion proteins beyond the Ena1B extensively used herein, the Ena1A (SEQ ID NO:1; FIG. 35 a) was used herein for fusion to Rubredoxin, based on the same principles as described herein above for Ena1B, with a fusion protein as shown in FIG. 35 b and with an amino acid sequence as present in SEQ ID NO: 185 for an insertion of Rubredoxin with single Glycine linker in the DE-Loop. The DE-loop of Ena1A of SEQ ID NO:1 corresponds to VGPGVSPANQI (SEQ ID NO: 186), and insertion of the MBP in the DE-loop was done between A65 and N66, though similar as for insertions in the Ena1B DE-loop that a Pro had to be removed, the N66 and Q67 were deleted by outward PCR when inserting an MBP, and also a glycine residue was added in the N- and C-terminal end of the insert to fuse with the Ena1A sequence. Primers used for this fusion construct are provided in table 5 (SEQ ID NO:188-191).

Alternatively, the Ena1A-HI-loop corresponds to TPATPIGT (SEQ ID NO:187), and insertion of the MBP in the HI-loop was done by substitution of the Pro with a Gly to avoid steric hindrance, and by inserting between the Thr-Gly and Ala residue of the loop by outward PCR when inserting an MBP, and a glycine linker residue was added in the C-terminal end of the insert to fuse with the remaining HI-loop Ena1A sequence (starting from Ala).

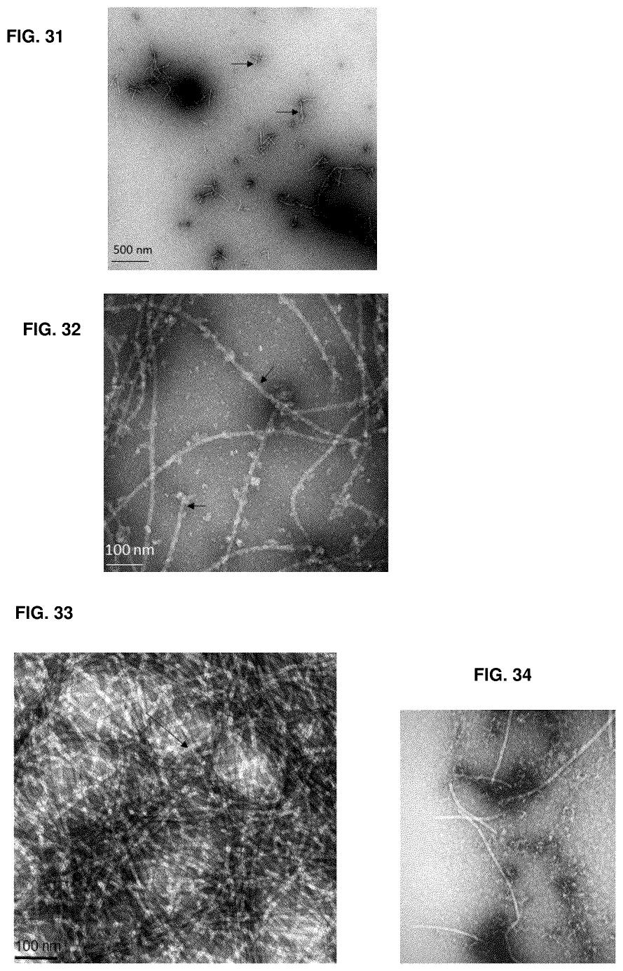

Cloning was done utilizing the same methods as used for the Ena1B-MBP fusions. Constructs were transformed into XjB strain (Derivative of BL21(DE3)-auto lysis strain, induced with arabinose to mildly express the lambda lysozyme into the cytoplasm, which becomes active after freeze-thaw of the harvested cells). Typically, a culture of 100 mL LB was grown and induced at an OD_{600 nm} of 0.8-1 with 1 mM IPTG, 1 mM arabinose for 5 h to overnight, at 30° C. The pellets were resuspended in 1×PBS, freeze-thawn, subsequently centrifuged for 40 min at 30 k×g after which the pellet was retained and subjected to a 1% SDS treatment followed by a centrifugation of 40 min at 30 k×g to obtain purified fibers in the pellet, which could be resuspended in milliQ water for negative stain TEM, imaged as shown in FIG. 31, indicating functionality in spontaneously folding into fibers.

### Example 14. Production and Fiber Assembly of Ena1B Inserted in the HI-Loop with MBPs of Table 1 and Further Metallothionein Proteins

As to test the fiber formation for fusions of Ena1B (SEQ ID NO:8) with metallothionein proteins, in particular with a cadmium-binding protein (SEQ ID NO:192; PDB 2MRB) and a zinc-binding protein (SEQ ID NO: 193; PDB 1AQQ), similar to the design and insertion in the HI-loop as described in previous examples, both constructs were made by genetic fusion of the metallothionein protein sequence with Ena1B in its HI-loop wherein the GTAAA was modified to GGGAA, between GGG and AA, additionally making use of small linker residues, SG and GG, for the N-/C-term of the insert resp., as shown in SEQ ID NO: 194 and 195. Transformation of the constructs was done into E. coli C43 (DE3) strain.

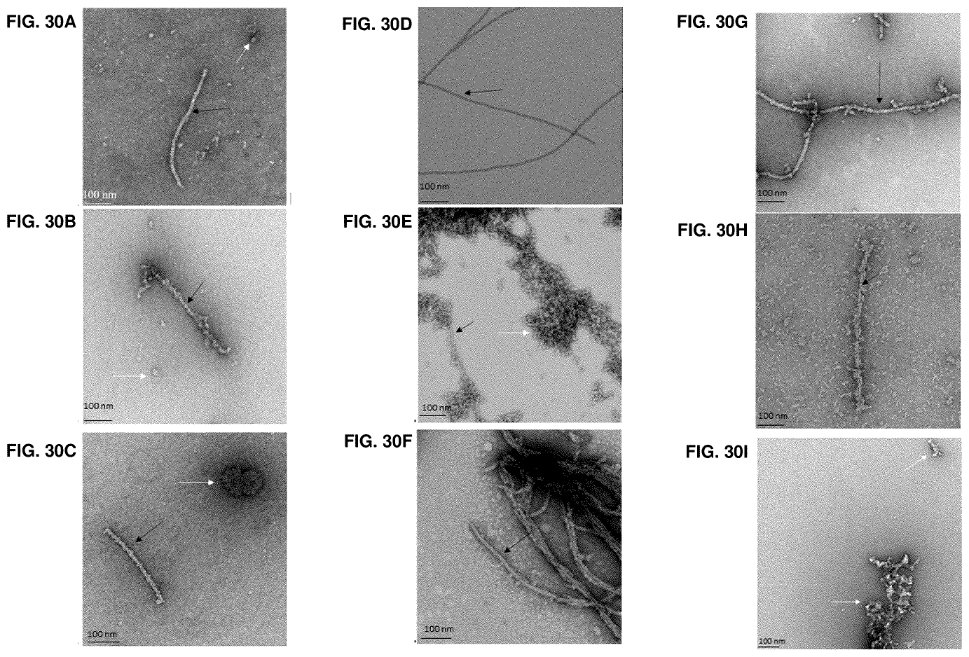

For these constructs, as well as for the Ena1B-HI-insertions as shown in Table 1, we produced the proteins by growing an overnight culture with 5 mL LB, 5 μL of 100 mg/ml kanamycin. A single colony was grown in 2 mL overnight, and this was added to 100 mL LB, 100 μL of 100 mg/ml kanamycin. When the culture reached an OD₆₀₀ of 0.8-1.3, induction was done with 0.5-1 mM IPTG. For Ena1B-HI-1AQQ and Ena1B-HI-2MRB, the addition of 1 mM ZnCl₂ and 1 mM CdCl₂ was added resp. 45 min after induction. Expression was done overnight at 20° C. After harvesting the cells, lysis buffer containing PBS, 5% DDM, 0.5 M NaCl, 10 mg/10 mL, 5 mM EDTA, 1 mM MgCl₂, DNAse (50 μg/ml), was used to resuspend the cells and lyse the cells, to further leave in a magnetic stirrer overnight. This suspension was centrifuged at 30,000 g for 45 min and the pellet was subsequently resuspended in 1% SDS and centrifuged again at the same conditions as previously. The resulting pellet was then resuspended in 1-3 ml ultra-pure water and used for negative stain imaging as shown in FIG. 30.

Although the initial production of most of the Ena1B-HI-MBP fusions resulted mainly in aggregates, by optimizing the production method we observed an increase in self-assembled fibers, so that the genetic fusion constructs as presented herein can be considered functional in allowing the Ena fiber to fold. The initial observation thus indicated that, upon recombinant production, the self-assembly of the Ena protein into aggregates or into fibers is also impacted by the process. For instance, increasing the OD (up to OD 2), and/or optimizing the IPTG concentration for inducing the expression may have an effect, as well as the incubation time and temperature.

### Example 15. Double Inserted Ena Fibers Using Ena1B-DE-HA Tag-HI-Rubredoxin Fusion Protein

To investigate the possibility of creating double insertions into Ena proteins and retaining their self-assembling nature, the addition of a HA-tag to Ena1B-HI-Rubredoxin was tested as a proof of principle.

The construct design was performed as described previously, resulting in the protein sequence as shown in SEQ ID NO:196. The protein was produced according to the method used in Example 14, and the fiber formation was analyzed upon negative stain imaging as shown in FIG. 32, indicating functional fibers.

### Example 16. Fine-Tuning of Linker Residues Between the Ena-Loop and the MBP Insertion is Key for Optimal Yield and Functionality

Finally, since, by serendipity, the Arginine was initially used for the Ena1B-DE-loop insertions (see Example 1-6), a reversion of the Ena1B-DE-Rubredoxin to the initially intended Gly-residue C-terminal linker was construed by PCR mutagenesis and produced as described above. So the proteins were identical besides the 1 linker residue being a glycine instead of arginine, the latter containing a charged, larger side chain (for comparison see SEQ ID NOs: 198 and 197, resp.).

Upon analyzing the isolated fibers by negative stain TEM, and as shown in FIG. 33 for the glycine-linker containing construct, the number of fibers seemed much higher when having a single glycine in the C-terminal end of the MBP, as compared to the Arg constructs previously used, and the tendency for aggregation is less apparent as well, suggesting that optimization of the linkers allows to tune the efficiency of fiber formation.

### Materials.

Competent cells were stored at −80° C. before transformation. After transformation, LB-glycerol (50%) stocks were prepared and stored at −80° C. Plasmids were stored at −20° C.

TABLE 4
Bacterial strains and plasmids used in this study.

| Competent cells | Genotype | Source |
|---|---|---|
| E. coli TOP10 | F- mcrA Δ(mrr-hsdRMS-mcrBC) Φ80lacZΔM15 | Invitrogen |
| | Δ lacX74 recA1 araD139 Δ(araleu)7697 galU | |
| | galK rpsL (StrR) endA1 nupG | |
| E. coli HST08 (Stellar) | F-, endA1, supE44, thi-1, recA1, relA1, gyrA96, | TakaraBio Europe, |
| | phoA, Φ80d lacZΔ M15, Δ(lacZYA-argF) U169, | France |
| | Δ(mrr-hsdRMS-mcrBC), ΔmcrA, λ- | |
| E. coli C43(DE3) | F - ompT hsdSB (rB- mB-) gal dcm (DE3) | (Miroux & Walker, 1996) |

| Plasmid | Description | Source |
|---|---|---|
| pET28a_Ena1B_nohis | Vector for the expression of Ena1B without a | Pradhan et al., (2020) |
| | His-tag | |
| pET28a_6xHis_TEV_Ena1B | Vector for the expression of Ena1B with a 6x | Pradhan et al., (2020) |
| | His-Tag with a TEV cleavage site | |

Small metal-binding proteins with one metal binding center were searched in the RCSB Protein databank (Berman, 2000). An internal databank of putative proteins from the Remaut Lab was also searched for small metal-binding proteins according to their predicted metal binding motifs. Of the selected ones, the distance between the N- and C-termini of the respective AlphaFold2 predictions were measured in PyMol′ and a protein Blast (States and Gish, 1994) was done to identify them. Identified proteins were named according to their accession numbers:
a. Rubredoxin from Desulfovivrio vulgaris (pdb: 2KKD): Rubredoxin
b. Protein PioC from Rhodopseudomonas palustris TIE-1 (pdb: 7a4L): HiPIP
c. Cold-shock protein from B. thuringiensis (Accession Nr: WP_098221117.1)
d. Hypothetical protein FC702_01375 from B. thuringiensis (Accession Nr: TKJ08955.1)
e. Hypothetical protein from B. thuringiensis (Accession Nr: WP_197262982.1)
f. Hypothetical protein from B. thuringiensis (Accession Nr: WP_000861196.1)
g. Hypothetical protein from B. thuringiensis (Accession Nr: WP_086405534.1)
h. Zinc ribbon domain-containing protein from B. thuringiensis (Accession Nr: WP_142338290.1)

Proteins with an N-to-C distance comparable to the dimension of the receiving ENA-loop were selected. For some proteins, N- and/or C-termini were truncated to lower the N-to-C distance. A synthetic DNA sequence (SEQ ID NO: 83) encompassing the concatenated coding sequences of the selected metal-binding sequences (in the order as provided in the list above) was ordered as one long sequence from Twist BioScience, USA and stored at −20° C.

Primers were designed with the help of SnapGene® and NEB® Tm Calculator, ordered from Integrated DNA Technologies (IDT, Leuven) and stored at −20° C.

TABLE 5
Primers for the insertion
of domains into recombinant Ena1B.

| | | SEQ ID |
|---|---|---|
| Primer | Nucleotide sequence (5′-3′) | NO: |
| DE3_open_f | GTGACCATCGTCTTTTAC | 84 |
| DE3_open_r | ACCCGTACCTGTATCCAC | 85 |
| HI_open_f | GCAGCGGAGACTGGTGAG | 86 |
| HI_open_r | GCCCAGAATAGTGACGGT | 87 |
| Rubredoxin_DE_fw | GATACAGGTACGGGTGGAAAATATGTTTGTACCGTTTGTAC | 88 |
| Rubredoxin_HI_fw | GTCACTATTCTGGGCGGAGGAAAATATGTTTGTACCGTTTGTAC | 89 |
| Rubredoxin_DE_rv | AAAGACGATGGTCACCCGCGCGGCCTCGAAC | 90 |
| Rubredoxin_HI_rv | ACCAGTCTCCGCTGCGCCCGCGGCCTCGAAC | 91 |
| HiPIP_DE_fw | GATACAGGTACGGGTGTCACCAAAAAGGCC | 92 |
| HiPIP_HI_fw | GTCACTATTCTGGGCGGAGTCACCAAAAAGGCC | 93 |
| HiPIP_DE_rv | AAAGACGATGGTCACCCGTGCTTTCCCTGCATAC | 94 |
| HiPIP_HI_rv | ACCAGTCTCCGCTGCGCCTGCTTTCCCTGCATAC | 95 |
| WP_098221117.1_DE_fw | GATACAGGTACGGGTGAACAGACTCCGGTG | 96 |
| WP_098221117.1_HI_fw | GTCACTATTCTGGGCGGAGAACAGACTCCGGTG | 97 |
| WP_098221117.1_DE_rv | AAAGACGATGGTCACCCGGAGGAGGCGCTCAC | 98 |
| WP_098221117.1_HI_rv | ACCAGTCTCCGCTGCGCCGAGGAGGCGCTCAC | 99 |
| FC702_01375_DE_fw | GATACAGGTACGGGTGGCTGCGTTCTCTGTCG | 100 |
| FC702_01375_HI_fw | GTCACTATTCTGGGCGGAGGCTGCGTTCTCTGTCG | 101 |
| FC702_01375_DE_rv | AAAGACGATGGTCACCCGGCCTTCGCAGTTGCAACC | 102 |
| FC702_01375_HI_rv | ACCAGTCTCCGCTGCGCCGCCTTCGCAGTTGCAACC | 103 |
| WP_197262982.1_DE_fw | GATACAGGTACGGGTGGCGTTGTGATCTGTCCCAA | 104 |
| WP_197262982.1_HI_fw | GTCACTATTCTGGGCGGAGGCGTTGTGATCTGTCCCAA | 105 |
| WP_197262982.1_DE_rv | AAAGACGATGGTCACCCGGCCTGATTCGATATTGCGAATTACAAAG | 106 |
| WP_197262982.1_HI_rv | ACCAGTCTCCGCTGCGCCGCCTGATTCGATATTGCGAATTACAAAG | 107 |
| WP_000861196_DE_fw | GATACAGGTACGGGTTCTGCAAAACTTTGTTTCTATTGTTTTAAAA | 108 |
| | TTC | |
| WP_000861196_HI_fw | GTCACTATTCTGGGCGGATCTGCAAAACTTTGTTTCTATTGTTTTA | 109 |
| | AAATTC | |
| WP_000861196_DE_rv | AAAGACGATGGTCACCCGGCCCAGCTCCTTATGACA | 110 |
| WP_000861196_HI_rv | ACCAGTCTCCGCTGCGCCGCCCAGCTCCTTATGACA | 111 |
| WP_086405534.1_DE_fw | GATACAGGTACGGGTGCATTAGTCTGTCCTAAATGCG | 112 |
| WP_086405534.1_HI_fw | GTCACTATTCTGGGCGGAGCATTAGTCTGTCCTAAATGCG | 113 |
| WP_086405534.1_DE_rv | AAAGACGATGGTCACCCGTAAGCGAATTTTGCAGC | 114 |
| WP_086405534.1_HI_rv | ACCAGTCTCCGCTGCGCCTAAGCGAATTTTGCAGC | 115 |
| WP_142338290.1_DE_fw | GATACAGGTACGGGTGCATCTCAGCTCTGTAGTTGTTG | 116 |
| WP_142338290.1_HI_fw | GTCACTATTCTGGGCGGAGCATCTCAGCTCTGTAGTTGTTG | 117 |
| WP_142338290.1_DE_rv | AAAGACGATGGTCACCCGGTCATGATGGGTGC | 118 |
| WP_142338290.1_HI_rv | ACCAGTCTCCGCTGCGCCGTCATGATGGGTGC | 119 |
| pETfw | GGTGATGCCGGCCACGATGCGTCC | 120 |
| pETrv | GGTTATGCTAGTTATTGCTCAGCG | 121 |
| T7_promoter_primer | TAATACGACTCACTATAGG | 122 |
| T7_terminator_primer | GCTAGTTATTGCTCAGCGG | 123 |
| Ena1A-open-DE-fw | ATTACGGTTACGGTTCTGGACTCC | 188 |
| Ena1A-open-DE-rv | GGCCGGGCTCACAC | 189 |
| Ena1A-Rubredoxin- | GGTGTGAGCCCGGCCggaAAATATGTTTGTACCGTTTGCGG | 190 |
| fragment-fw | | |
| Ena1A-Rubredoxin- | AACCGTAACCGTAATtccCGCGGCCTCGAAC | 191 |
| fragment-rv | | |

Purification of PCR samples were done using Nucleospin Gel and PCR clean-up kit (Macherey-Nagel). Purification of plasmids was performed using the GeneJet Plasmid Miniprep Kit (Fermentas, USA).

Molecular weight ladders were stored at −20° C. The SmartLadder 200 bp-10 kb (Eurogentec) was stored at 4° C. The Generuler 1 kb (Thermoscientific, USA) was used for all plasmid DNA samples in 1% agarose electrophoresis. The SmartLadder 200 200 bp-10 kb (Eurogentec) was used for the detection of plasmid DNA samples in 1% agarose electrophoresis. The Generuler 50 bp (Thermofisher, USA) was utilized for the detection of insert DNA in 1-2% agarose electrophoresis. The Pageruler Prestained Protein Ladder (Thermoscientific, USA) was used as a protein ladder on SDS-gels.

TABLE 6
List of media and reagents used herein.

| Component/Reagents | Source |
|---|---|
| Lysogeny Broth (LB) | Duchefa Biochemie, The Netherlands |
| Micro Agar | Duchefa Biochemie, The Netherlands |
| Super Optimal Broth with Catabolite repression (SOC) | TakaraBio Europe, France |
| Kanamycin | Duchefa Biochemie, The Netherlands |
| Lysozyme | Sigma Aldrich, USA |
| B-mercaptoethanol | Sigma Aldrich, USA |
| Isopropyl-beta-D-thiogalactopyranoside (IPTG) | Inalco S.p.A., Italy |
| Fast Digest Dpnl | Thermo Fischer, USA |
| Sodium dithionite | Sigma Aldrich, USA |
| Methylene blue | Sigma Aldrich, USA |
| LDS Sample Buffer (NuPAGE) | Life Technologies, USA |
| Instant blue | Gentaur, Belgium |
| NaH₂PO₄ | Carl Roth, Belgium |
| Na₂HPO₄ | Carl Roth, Belgium |
| NaCl | Sigma Aldrich, USA |
| Tris | Sigma Aldrich, USA |
| Glacial acetic acid | Sigma Aldrich, USA |
| Ethylenediaminetetraacetic acid (EDTA) | Sigma Aldrich, USA |
| Tris-Base | Sigma Aldrisch, USA |
| Methanol | Honeywell Reod |
| Glycine | Carl Roth, Belgium |
| Sodium dodecyl sulphate (SDS) | Sigma Aldrich, USA |
| MgCl₂ | vwr chemicals, USA |
| Dodecyl-β-maltoside | Inalco S.p.A., Italy |
| Agarose | Lonza, USA |
| Midori green | Nippon Genetics Europe GmbH, |
| | Germany |
| Bisacrylamide | Severn Biotech, UK |
| Ammonium persulphate (APS) | Bio-rad Laboratories, Belgium |
| N,N,N′N′-teramethylethylenediamine (TEMED) | Sigma Aldrich, USA |
| BCIP/NBT | Roche Diagnostics, Belgium |

TABLE 7
General buffers used herein.

| Solution | Components |
|---|---|
| 10x Phosphate Buffer Saline (PBS) | 38 mM NaH2PO4, 162 mM Na2HPO4, 1.67 mM NaCl |
| 10x TAE-buffer | 40 mM Tris, 20 mM glacial acetic acid, 1 mM EDTA pH 8.4 |
| Western blot Transfer buffer | For 1 L: 3 g Tris-Base, 200 mL MeOH, 14.4 g glycine, 10 |
| | mL 10% SDS |
| Leammli Running buffer (10x) | For 1 L: 30.3 g Tris base, 144.2 g Glycine, 10 g sodium |
| | dodecyl sulphate (SDS) |
| Western blot development buffer | 20 mM Tris pH 9.5, 100 mM NaCl, 50 mM MgCl2, pH 9.5 |

### Methods

### Polymerase Chain Reaction (PCR)

Three types of PCRs were performed: the plasmid was linearized and amplified with the help of Phusion or Primestar Max. The insert was amplified and overhangs complementary to the plasmid was added with the Phusion or Primer Max. Colony PCR was done to screen positive colonies after insertion to the plasmid with the help of ExTaq Polymerase or DreamTaq Polymerase.

Phusion polymerase is a high-fidelity polymerase used for the amplification of vectors with an error rate of 4.4.10⁻⁷ in Phusion HF buffer. For 50 μL PCR, the reaction mix consists of: 10 μL Buffer HF (Finnzymes), 1 μL 2.5 mM dNTP's, 33.5 μL filtered water, 2.5 μL 10 μM forward (Fw) Primer, 2.5 μL 10 μM reverse (Rv) Primer, 1 μL template, 0.5 μL Hot Phusion Start DNA Polymerase. The following PCR program was used: Initial denaturation: 95° C., 5 min; 30 cycles of: Denaturation: 95° C., 40 s/Annealing of primes: 52° C., 40 s/Extension: 72° C., 1 min; Final extension: 72° C., 4 min.

Primestar Max is a faster and higher fidelity polymerase used for the amplification of vectors with an error rate of 0.00108%. For 40 μL of PCR, the reaction mix was: 1 μL template, 1 μL 10 μM forward and reverse primer, 20 μL Primestar Mix, 16 μL filtered water. The following PCR program was used: 30 cycles: Denaturation: 98° C., 10 s/Annealing: 55-68° C., 5 s/Extension: 72° C., 5 s to 1 min.

ExTaq Polymerase: for 20 μL of PCR product, the following products were utilized: 0.2 μL 5 units/μL TaKaRa ex Taq, 2 μL 10 Ex Taq Buffer, 15 μL filtered water, 1 μL 10 μM forward and reverse primer. The following PCR program was used: Initial denaturation: 98° C., 4 min; 30 cycles: Denaturation at 98° C. for 30 s/Annealing at 52° C. for 40 s/Elongation at 72° C. for 3 min; Final elongation at 72° C. for 1 min.

DreamTaq Polymerase: for 20 μL of PCR product, the following reaction mix was used: 8 μL filtered water, 0.5-1 μL of 10 μM fw and rv primer, 10 μL DreamTaq Mix, 0.4 μL template or a prick of a colony. The following PCR program was used: Initial denaturation: 95° C.-3 min; 30-35 cycles: Denaturation: 95° C., 30 s/Annealing: 55° C., 30 s/Elongation: 72° C.-1 min; Final Elongation at 72° C.-5 min.

### Agarose Electrophoresis

Polymerase chain reactions (PCR) were analyzed by loading 5 μL of PCR sample on a 1-2% agarose gel depending on the size of the expected PCR products. DNA bands were visualized using Midori green. Molecular ladders were used as a reference and a voltage of 110 V was applied for 25 min.

### Dpnl Digestion

By adding 1/20 dilution fast Digest Dpn/and 1/10 Buffer to PCR products and incubating it at 37° C. for 1 h, Dpnl cleaved methylated DNA templates which originate from bacterial plasmids.

Preparation and Transformation of E. coli CaCl₂)-Competent Cells

An overnight culture was made by inoculating 5 ml LB medium with a colony of TOP10 or C43 (DE3) cells. 4 mL overnight culture was used to inoculate 200 mL LB-medium. The cells were grown at approximately 100 rpm shaking conditions and 37° C. until the OD₆₀₀ was between 0.4 and 0.6. The cells were cooled for 15 min at 4° C. and kept on ice afterwards. In four precooled falcons, pellets were obtained through centrifugation in a centrifuge 5430 (EPpendorf, Germany) at 1057 g in an F-35-6-30 rotor during 4 min. The pellets were resuspended in cold sterile 0.1 M CaCl₂) and pooled. After 25 min incubation, the cells were centrifuged and resuspended in 2 mL 0.1 M CaCl₂) and 400 μL 80% glycerol. These were aliquoted in pre-cooled EPpendorf's (50 μL), flash frozen, and stored at −80° C.

The transformation of CaCl₂)-competent cells was done by thawing an aliquot of 50 μL competent cells on ice. After adding 1 μL of plasmid and 2-5 μL insert, the suspension was mixed by flicking the tube and placing it back in ice. Incubation was done for 20 min and a heat shock was performed at 42° C. for 45 s. Afterwards, the cells were placed on ice for 2 min, when 100 μL SOC or LB medium was added. Phenotypic expression was done by placing the cells in the incubator at 37° C. for 40 min to 2 h. 100-150 μL was poured on LB agar plates containing 100 μg/ml kanamycin and spread using glass beads. The plates were incubated overnight at 37° C.

### Small-Scale Recombinant Expression and Purification of Endospore Appendages

A 5 mL overnight preculture was made by inoculating 5 mL LB medium containing 100 μg/mL kanamycin with one colony of CD43 (DE3) (pET28a_Ena1B_nohis). This preculture was used to inoculate a 5 mL LB containing 100 μg/mL kanamycin (1:50 dilution). Cultures were grown at 37° C. with 140 rpm shaking and induced with 1 mM IPTG when OD₆₀₀ was between 0.6 and 0.8. After induction, cultures were placed at 30° C. and 100-150 rpm shaking for 3 h. Alternatively, induction and subsequent expression was also done at 20° C. overnight at 130 rpm shaking in order to minimize inclusion body production.

Cultures were centrifuged for 15 min at 5 000-14 000 rpm with rotor JA 14.50 (Beckman Coulter, Avanti J-20 centrifuge, Belgium) and pellets resuspended in lysis buffer containing 1×PBS, 1% Dodecyl-β-maltoside (DDM), 1 mg/ml lysozyme, 0.5 M NaCl, 5 mM EDTA in a volume 10% of the culture volume. This was left overnight at 37° C. or at room temperature. Cell lysates were centrifuged at 14 000 rpm for 30 min. The same volume MQ water as lysis buffer was added to the pellet and homogenized using Yellow-line OST 20 (imLab, Belgium).

Alternatively, cultures were centrifuged for 15 min at 5 000 rpm with rotor JA 14.50 (Beckmann Coulter, Avanti J-20 centrifuge). Afterwards, 1% (w/v) sodium dodecyl sulphate (SDS) was added to each pellet and incubated for 25 min in 95° C. This was centrifuged at 20813 rpm in a for F-35-6-30 rotor in a Centrifuge 5430 (EPpendorf, Germany) for an hour and pellets were washed with in MQ water and centrifuged at 20813 rpm for 30 min to rid of SDS.

### Negative Stain Electron-Microscopy

5 μL aliquots of recombinant S-ENA were applied onto a copper grid with small meshes. Excess liquid was removed from the grid via side blotting with Whatman paper and the grid was washed twice with 20 μL MQ. In the last step, the grid was incubated for 5 sec in 2% uranyl acetate as stain (Histo-Line Laboratories, Italy). These samples were then visualized with the JEM-1400 Transmission Electron Microscope (JEOL) with a CMOS Image Sensor in BECM, Vrije Universiteit Brussel (VUB).

### Preparation of Cryo-TEM Grids and Cryo-EM Data Collection

Holey copper grids (QUANTIFOIL®) mesh grids with 2 μm holes and 1 μm spacing were glow discharged in vacuum with plasma current of 5 mA for 1 min. Cryo-plunging was done in a Gatan CP3 cryo plunger at room temperature by pipetting 3 μL protein sample on the grids at 100% humidity. The grid was machine blotted with grade 2 Whattman paper on both sides. Hereafter, the grid was plunged into 180° K liquid ethane and transferred to a grid box and stored in liquid nitrogen for further use. High-resolution cryogenic electron microscope (CryoEM) 2D micrograph movies were collected on a JEOL Cryoarm3000 microscope with an energy filter and a K3 gatan camera detector with an aperture of 100 microns, pixel size of 0.76 Å/pxl, 300 keV and an exposure of 64.6 e⁻/Å2 taken over 60 frames/image.

2D-Classification and 3D-Reconstruction with CryoSPARC™.

4300 raw movies (.tif) were imported into CryoSPARC™ with parameters: 0.766 Å/pixel, accelerating voltage of 300 keV and a total exposure dose of 64.6 e⁻/Å2. Patch Motion Correction was used to correct for stage drift as well as beam induced anisotropic motion. Thusly obtained motion corrected micrographs (.mrc) went through Contrast Transfer Function (CTF)-estimation to determine the image defocus and astigmatism and to estimate the resolution of the raw images. Next, a 5 Å cut-off was introduced, i.e., motion corrected images with an estimated resolution <5 Å were not retained for further analysis. Template-free filament picking was done using the ‘filament tracer’ job searching for helical segments of 110 Å diameter and an inter-box distance of 5.6 nm. Based on this picking, helical segments were extracted and twofold binned to a box size of 200×200 pixels (380 Å×380 Å) and an inter-box distance of 5.6 nm, yielding an initial particle stack of 500600 particles. To filter out low resolution particles, 2D-classification was performed, yielding 2D-class averages were yielded of Ena1B-HI-Rubredoxin fiber segments. At this point, only particles that correspond to a class average with a reported resolution of 5 Å were retained for further analysis. Next, helical refinement was performed using a featureless cylinder of 110 Å diameter as an initial model taking input rise and twist values from the WT recEna1B structure (pdb: 7A02), From this, one obtained a 3D reconstructed volume of the Ena1B-HI-Rubredoxin helix at 3.11 Å. Next, unbinned particles were re-extracted and recentered using the offsets and helical refinement was reinitiated using the preliminary 3D volume. This yielded a reconstructed volume at 2.30 Å with a refined helical rise and twist values of 3.15 Å and 31.1 degrees.

An atomic model of the Ena1B_HI_RR helix was built using Wincoot, and models 2KKD (rubredoxin) and 7A02 (S-ENA WT structure) as structural templates. The 2KKD chain was docked into the volume and the connecting loops to Ena1B were built manually, i.e., residues of inserts and loop to C′ terminal of Ena1B were renumbered. The chain ID of the insert was changed to the Chain ID of Ena1B to urge WinCoot to recognize the molecules as one molecule. Linkers were added onto rubredoxin with “add residue”. “Real Space Refine Zone” linked the two molecules and fitted them into the map for rounds of refinements. After each round of refinement, each residue in the monomer was manually inspected. Once a monomer was well fitted into its map, the monomer was multiplied to gain a recombinant Ena1B fiber.

### Sodium Dodecyl Sulphate Polyacrylamide Gel Electrophoresis (SDS-PAGE) and Western Blotting.

To analyze the presence of oligomeric and monomeric recombinant ENAs on SDS-PAGE and Western blot, aliquots of 40 μL were subjected to 1% (w/v) SDS with and without 1 μL 20% (v/v) beta-mercaptoethanol and heated at 100° C. for 15 min.

Aliquots of 30 μL purified recombinant ENA's were mixed with 10 μL of 4×LDS Sample Buffer. These were incubated at 100° C. for 5 min, centrifuged, and 10 μL loaded on a 20% polyacrylamide gel. 5 μL of PageRuler Prestained Ladder was used as a reference. For 45 min, 230 V was applied to the gel. Hereafter, the gel was stained with the help of Instant Blue.

Proteins from the SDS-gel were blotted onto a methanol-activated PVDF-membrane (Thermoscientific, USA) using a TransBlot Turbo Transfer System (Bio-Rad Laboratories, Belgium) and six Whatman papers soaked in Transfer buffer. A voltage of 25 V was applied for 12 min.

Western blots were performed using the iBind™ Western System, 1× iBind™ Solution was made and spread evenly across the flow region on the iBind™ Card. The blotted PVDF-membrane was afterwards immersed in 5 ml of previously made 1× iBind™ Solution and placed in the iBind™ Western System (ThermoScientific, USA) with the protein side down. The lid was then closed. Diluted Rabbit anti-Ena1B and anti-rabbit alkaline phosphate antibody solutions were made to a dilution of 1:1000. Antibody and washing solutions were added in wells of the closed iBind™ Western System in the following order: 2 mL rabbit anti-ENA solution (1:500 dilution), 2 mL iBind™ Solution, 2 mL anti-rabbit alkaline phosphate antibody solution (1:1000 dilution) and 6 mL 1× iBind™ Solution. After three hours, the Western blot was developed using 10 ml development buffer with 50 μL BCIP/NBT substrate for 5 min. After developing, the membrane is washed with MQ water.

### Spectroscopic Analyses

With the NanoDrop One (ThermoScientific, USA), UV-Vis spectra were measured with 10 mM Tris pH 7 as blank. 490 μL ENA-rubredoxin was reduced by adding an excess of sodium dithionite (10 mM). Spectra of oxidized (495 μL) and reduced ENA-rubredoxin buffered with 10 mM Tris at pH 7 were measured. Analysis was done with GraphPad Prism®.

Release and captures of metals were done by incubating Ena1B-HI-Rubredoxin with 10 mM sodium dithionite at 65° C. for 30 min. It was centrifuged for 30 min in a F-35-6-30 rotor in a centrifuge 5430 (EPpendorf, Germany) and an excess of NiCl₂ (100 μM) was added to the pellet. After 15 min, it was centrifuged again at the same conditions and a pellet was retrieved.

### Redox Titration-Spectroscopic Analysis

The redox potential of Ena1B-HI-Rubredoxin was measured with methylene blue as indicator. Concentration of methylene blue was so that the absorbance of methylene blue at 668 nm was the same as the absorbance of Ena1B-HI-Rubredoxin at 480 nm. Five concentrations of sodium dithionite were made. (1M, 0.1 M, 0.01 M, 0.001 M). 0.1 μL of each of the five concentrations of sodium dithionite were added to aliquots of 9.9 μL of Ena1B-HI-Rubredoxin and methylene blue mixture. UV-Vis spectra were then measured with the NanoDrop One (0). Further analysis was done on the two nearest concentrations where the absorbance of wavelengths 490 nm and 668 nm changed drastically. 20 new concentrations of sodium dithionite were made (0.111 M, 0.125 M, 0.143 M, 0.1667 M, 0.2 M, 0.25 M, 0.3 M, 0.33 M, 0.4 M, 0.5 M, 0.6 M, 0.67 M, 0.70 M, 0.75 M, 0.8 M, 0.83 M, 0.86 M, 0.875 M, 8.89 M, and 0.9 M). 0.1 μL of these concentrations of sodium dithionite were added to aliquots of 9.9 μL of Ena1B-HI-Rubredoxin and methylene blue mixture again. 0.2 μL of 1M sodium dithionite was added as well to a 9.9 UL Ena1B-HI-Rubredoxin-methylene blue mixture and a fully reduced solution is achieved by adding an excess of 1 M sodium dithionite. UV-Vis spectra were then measured with the NanoDrop One, making sure to measure as soon as sodium dithionite is added to the Ena1B-HI-Rubredoxin mixture.

Analysis is done using GraphPad Prism® and Microsoft Excel®. The following analysis was done with the help of (Efimov et al., 2014).

Influence of the absorbance of methylene blue was removed by subtracting the absorbance of methylene blue at 490 nm. Reduction potential of Ena1B-HI-Rubredoxin was calculated using the following equation:

$$Equation 1 : Nernst equation with R : gas constant , \\ n : number of electrons transferred , F : Faraday ’ s constant , \\ T : Temperature in Kelvin . The Nernst concentration \\ term is known as \frac{RT}{nF} ln ( \frac{[ {P}_{ox} ]}{[ {P}_{red} ]} )$$

$${E}_{P} = {E}_{m . P} + \frac{RT}{n F} ln ( \frac{[ {P}_{ox} ]}{[ {P}_{red} ]} ) .$$

Where E_{P} is the measured potential of Ena1B-HI-Rubredoxin, E_{m,P} is the reduction potential to be calculated. Since electrochemical potentials are the same at equilibrium, the following equation determined E_{P}:

$$\begin{matrix}Equation 2 : Relation between {E}_{D} and {E}_{P} with D : Dye and P : Protein & \\ {E}_{m , D} + \frac{R T}{n F} ln ( \frac{[ {D}_{o x} ]}{[ {D}_{r e d} ]} ) = {E}_{m , P} + \frac{R T}{n F} ln ( \frac{[ {P}_{o x} ]}{[ {P}_{r e d} ]} ) . & \end{matrix}$$

The next equation was used to determine the ratio of oxidized and reduced concentrations of both Ena1B-HI-Rubredoxin and methylene blue.

$$Equation 3 : Relation between the ratio of oxidized and reduced \\ concentrations of Ena1B - HI - Rubredoxin and methylene blue with \\ measured absorbance values . Absorbances are measured at \\ 668 nm for methylene blue and 490 nm for Ena1B - HI - Rubredoxin$$

$$\frac{A - {A}_{min}}{{A}_{max} - A} = \frac{[ oxidised ]}{[ reduced ]} .$$

A Nernst Plot was made by plotting the Nernst concentration term for methylene blue against the Nernst concentration term for Ena1B-HI-Rubredoxin. The Y-intercept of the Nernst Plot was subtracted by the mid-point potential of the dye, which calculated the mid-point potential of the protein. This was according to the following equation:

$$\begin{matrix}Equation 4 : Fitted linear regression of Nernst Plot & \\ y = ( {E}_{m , P} - {E}_{m , D} ) + x . & \end{matrix}$$

### X-Ray Fluorescence

From aliquots of cell lysate, 1% (w/v) sodium dodecyl sulphate (SDS) was added and incubated at 100° C. for 30 min. The suspension is subsequently centrifuged for 30 min in a F-35-6-30 rotor in a centrifuge 5430 (EPpendorf, Germany). Pellets were washed with MQ water and centrifuged again for 30 min in a F-35-6-30 rotor in a centrifuge 5430 (EPpendorf, Germany). Metal containing protein samples were fished with a loop and flash cooled. A metal free protein sample was added as control. X-ray fluorescence analysis was performed at the Proximal beamline of the Soleil synchrotron facility according to a protocol described by (Handing et al., 2018).

Sequence Listing

>SEQ ID NO: 1: Bacillus cereus NVH 0075-95 383 Endospore appendage
(Ena) 1A amino acid sequence (GenBank Protein ID: KMP91697.1; 126 aa)

| | | |
|---|---|---|
| >SEQ ID NO: 2: GCF_007673655.1 Ena1A | 125 | B. mycoides |
| >SEQ ID NO: 3: GCF_002251005.2_Ena1A | 126 | B. cytotoxicus |
| >SEQ ID NO: 4: GCF_001884105.1_Ena1A | 125 | B. luti |
| >SEQ ID NO: 5: GCA_000171035.2_Ena1A | 126 | B. cereus |
| >SEQ ID NO: 6: GCF_007682405.1_Ena1A | 126 | B. tropicus |
| >SEQ ID NO: 7: GCF_002572325.1_Ena1A | 126 | B. wiedmannii |

>SEQ ID NO: 8: Bacillus cereus NVH 0075-95 383 Endospore appendage
(Ena) 1B amino acid sequence (GenBank Protein ID: KMP91698.1; 117 aa)

| | | |
|---|---|---|
| >SEQ ID NO: 9: GCF_000161255.1_Ena1B | 120 | B. cereus |
| >SEQ ID NO: 10: GCF_900095655.1_Ena1B | 116 | B. cytotoxicus |
| >SEQ ID NO: 11: GCA_000171035.2_Ena1B | 117 | B. cereus |
| >SEQ ID NO: 12: GCF_002572325.1_Ena1B | 117 | B. wiedmannii |
| >SEQ ID NO: 13: GCF_001884105.1_Ena1B | 117 | B. luti |
| >SEQ ID NO: 14: GCF_007682405.1_Ena1B | 117 | B. tropicus |

>SEQ ID NO: 15: Bacillus cereus NVH 0075-95 383 Endospore appendage
(Ena) 1C amino acid sequence (GenBank Protein ID: KMP91699.1; 155 aa)

| | | |
|---|---|---|
| >SEQ ID NO: 16: GCF_900094915.1_Ena1C | 150 | B. cytotoxicus |
| >SEQ ID NO: 17: GCF_000789315.1_Ena1C | 155 | B. cereus |
| >SEQ ID NO: 18: GCF_001044745.1_Ena1C | 155 | B. wiedmannii |
| >SEQ ID NO: 19: GCF_002568925.1_Ena1C | 155 | B. wiedmannii |
| >SEQ ID NO: 20: GCF_001884105.1_Ena1C | 155 | B. luti |

>SEQ ID NO: 21: Bacillus cytotoxicus NVH 391-98 Endospore appendage
(Ena) 2A amino acid sequence (GenBank Protein ID: ABS21009.1; 126 aa)

| | | |
|---|---|---|
| >SEQ ID NO: 22: GCF_002555305.1_Ena2A | 122 | B. wiedmannii |
| >SEQ ID NO: 23: GCF_000712595.1_Ena2A | 119 | B. manliponensis |
| >SEQ ID NO: 24: GCF_000008005.1_Ena2A | 122 | B. cereus |
| >SEQ ID NO: 25: GCF_000161275.1_Ena2A | 122 | B. cereus |
| >SEQ ID NO: 26: GCF_000007845.1_Ena2A | 122 | B. anthracis |
| >SEQ ID NO: 27: GCF_002589195.1_Ena2A | 122 | B. toyonensis |
| >SEQ ID NO: 28: GCF_000290695.1_Ena2A | 122 | B. mycoides |

>SEQ ID NO: 29: Bacillus cytotoxicus NVH 391-98 Endospore appendage
(Ena) 2B amino acid sequence (GenBank Protein ID: ABS21010.1; 117 aa)

| | | |
|---|---|---|
| >SEQ ID NO: 30: GCF_002555305.1_Ena2B | 113 | B. wiedmannii |
| >SEQ ID NO: 31: GCF_000712595.1_Ena2B | 114 | B. manliponensis |
| >SEQ ID NO: 32: GCF_000008005.1_Ena2B | 112 | B. cereus |
| >SEQ ID NO: 33: GCF_000803665.1_Ena2B | 110 | B. thuringiensis |
| >SEQ ID NO: 34: GCF_004023375.1_Ena2B | 111 | B. mycoides |
| >SEQ ID NO: 35: GCF_000742875.1_Ena2B | 114 | B. anthracis |
| >SEQ ID NO: 36: GCF_002589605.1_Ena2B | 114 | B. toyonensis |
| >SEQ ID NO: 37: GCF_900095005.1_Ena2B | 114 | B. mycoides |

>SEQ ID NO: 38: Bacillus cytotoxicus NVH 391-98 Endospore appendage
(Ena) 2C amino acid sequence (GenBank Protein ID: ABS21011.1; 150 aa)

| | | |
|---|---|---|
| >SEQ ID NO: 39: GCF_000338755.1_Ena2C | 135 | B. thuringiensis |
| >SEQ ID NO: 40: GCF_003386775.1_Ena2C | 135 | B. mycoides |
| >SEQ ID NO: 41: GCF_002578975.1_Ena2C | 135 | B. wiedmannii |
| >SEQ ID NO: 42: GCF_006349595.1_Ena2C | 135 | B. pacificus |
| >SEQ ID NO: 43: GCF_001455345.1_Ena2C | 134 | B. thuringiensis |
| >SEQ ID NO: 44: GCF_004023375.1_Ena2C | 144 | B. mycoides |
| >SEQ ID NO: 45: GCF_003227955.1_Ena2C | 136 | B. anthracis |
| >SEQ ID NO: 46: GCF_001317525.1_Ena2C | 136 | B. wiedmannii |
| >SEQ ID NO: 47: GCF_000712595.1_Ena2C | 145 | B. manliponensis |
| >SEQ ID NO: 48: GCF_007673655.1_Ena2C | 139 | B. mycoides |

>SEQ ID NO: 49: Bacillus (multispecies-Bacillus cereus ATCC10987-
GCF_000008005.1) Endospore appendage (Ena) 3A amino acid sequence
(WP_017562367.1; 113 aa)
>SEQ ID NO: 50: WP_157293150.1/1-112 DUF3992 domain-containing protein
[Bacillus sp. ms-22]
>SEQ ID NO: 51: WP_105925236.1/1-114 DUF3992 domain-containing protein
[Bacillus sp. LLTC93]
>SEQ ID NO: 52: OLP66313.1/1-115 hypothetical protein BACPU_06150
[Bacillus pumilus]
>SEQ ID NO: 53: WP_010787618.1/1-115 DUF3992 domain-containing protein
[Bacillus atrophaeus]
>SEQ ID NO: 54: WP_040373377.1/1-116 DUF3992 domain-containing protein
[Peribacillus psychrosaccharolyticus]
>SEQ ID NO: 55: WP_091498261.1/1-115 DUF3992 domain-containing protein
[Amphibacillus marinus]
>SEQ ID NO: 56: WP_008633630.1/1-115: DUF3992 domain-containing protein
[Bacillaceae]
>SEQ ID NO: 57: WP_124051031.1/1-116 DUF3992 domain-containing protein
[Bacillus endophyticus]
>SEQ ID NO: 58: WP_049679853.1/1-114 DUF3992 domain-containing protein
[Peribacillus loiseleuriae]
>SEQ ID NO: 59: WP_062184382.1/1-118 DUF3992 domain-containing protein
[Bacillales]
>SEQ ID NO: 60: WP_049681018.1/1-118 DUF3992 domain-containing protein
[Peribacillus loiseleuriae]
>SEQ ID NO: 61: WP_154975023.1/1-118 DUF3992 domain-containing protein
[Bacillus megaterium]
>SEQ ID NO: 62: WP_048022205.1/1-118 DUF3992 domain-containing protein
[Bacillus aryabhattai]
>SEQ ID NO: 63: WP_036199318.1/1-114 DUF3992 domain-containing protein
[Lysinibacillus sinduriensis]
>SEQ ID NO: 64: MQR85259.1/1-115 DUF3992 domain-containing protein
[Bacillus megaterium]
>SEQ ID NO: 65: WP_111616476.1/1-114 DUF3992 domain-containing protein
[Bacillus sp. YR335]
>SEQ ID NO: 66: TDL84647.1/1-113 DUF3992 domain-containing protein
[Vibrio vulnificus]
>SEQ ID NO: 67: WP_119116371.1/1-114 DUF3992 domain-containing protein
[Peribacillus asahii]
>SEQ ID NO: 68: WP_000057858.1/1-116 DUF3992 domain-containing protein
[Bacillus cereus]
>SEQ ID NO: 69: WP_000192611.1/1-114 DUF3992 domain-containing protein
[Bacillus cereus]
>SEQ ID NO: 70: WP_000057857.1/1-114 MULTISPECIES: DUF3992 domain-
containing protein [Bacillus cereus group]
>SEQ ID NO: 71: WP_035510401.1/1-114 MULTISPECIES: DUF3992 domain-
containing protein [Halobacillus]
>SEQ ID NO: 72: WP_101934191.1/1-114 DUF3992 domain-containing protein
[Virgibacillus dokdonensis]
>SEQ ID NO: 73: WP_149173096.1/1-114 DUF3992 domain-containing protein
[Bacillus sp. BPN334]
>SEQ ID NO: 74: AAS42063.1/1-115 hypothetical protein BCE_3153
[Bacillus cereus ATCC 10987]
>SEQ ID NO: 75: WP_100527630.1/1-114 DUF3992 domain-containing protein
[Paenibacillus sp. GM1FR]
>SEQ ID NO: 76: WP_026691041.1/1-115 DUF3992 domain-containing protein
[Bacillus aurantiacus]
>SEQ ID NO: 77: WP_102693317.1/1-113 DUF3992 domain-containing protein
[Rummeliibacillus pycnus]
>SEQ ID NO: 78: WP_071391073.1/1-109 DUF3992 domain-containing protein
[Anaerobacillus alkalidiazotrophicus]
>SEQ ID NO: 79: WP_107839371.1/1-111 DUF3992 domain-containing protein
[Lysinibacillus meyeri]
>SEQ ID NO: 80: WP_066166707.1/1-111 DUF3992 domain-containing protein
[Metasolibacillus fluoroglycofenilyticus]
>SEQ ID NO: 81: Ena2A amino acid sequence Bacillus thuringiensis
(WP_001277540.1)
>SEQ ID NO: 82: Ena2C amino acid sequence Bacillus thuringiensis
(WP_014481960.1)
>SEQ ID NO: 83: synthetic DNA containing coding sequences of Metal-
binding proteins (MBPs) of SEQ ID NO: 129-136.
>SEQ ID NOs: 84-123, 188-191: Primers of table 5
>SEQ ID NO: 124: Ena1B (SEQ ID NO: 8) DE-loop amino acid sequence
>SEQ ID NO: 125: Ena1B truncated DE-loop (without ending Pro)
>SEQ ID NO: 126: engineered Ena1B DE-loop (Pro substituted with X,
wherein X is Arg or Gly, for inserting an MBP)
>SEQ ID NO: 127: Ena1B (SEQ ID NO: 8) HI-loop amino acid sequence
>SEQ ID NO: 128: engineered Ena1B HI-loop (TA substituted with GG)
>SEQ ID NO: 129: Rubredoxin insert
>SEQ ID NO: 130: HiPIP insert
>SEQ ID NO: 131: WP_098221117.1 insert
>SEQ ID NO: 132: FC702_01375 insert
>SEQ ID NO: 133: WP_197262982.1 insert
>SEQ ID NO: 134: WP_000861196 insert
>SEQ ID NO: 135: WP_086405534.1 insert
>SEQ ID NO: 136: WP_142338290.1 insert
>SEQ ID NO: 137: nucleotide sequence of Ena1B lacking the DE loop
>SEQ ID NO: 138: amino acid sequence of Ena1B lacking the DE loop
>SEQ ID NO: 139: nucleotide sequence of Ena1B lacking the HI loop
>SEQ ID NO: 140: amino acid sequence of Ena1B lacking the HI loop
>SEQ ID NO: 141: Rubredoxin with Ena1B linker/DE-loop insert
>SEQ ID NO: 142: Rubredoxin with Ena1B linker/HI-loop insert
>SEQ ID NO: 143: HiPIP with Ena1B linker/DE-loop insert
>SEQ ID NO: 144: HiPIP with Ena1B linker/HI-loop insert
>SEQ ID NO: 145: WP_098221117.1 with Ena1B linker/DE-loop insert
>SEQ ID NO: 146: WP_098221117.1 with Ena1B linker/HI-loop insert
>SEQ ID NO: 147: FC702_01375 with Ena1B linker/DE-loop insert
>SEQ ID NO: 148: FC702_01375 with Ena1B linker/HI-loop insert
>SEQ ID NO: 149: WP_197262982.1 with Ena1B linker/DE-loop insert
>SEQ ID NO: 150: WP_197262982.1 with Ena1B linker/HI-loop insert
>SEQ ID NO: 151: WP_000861196 with Ena1B linker/DE-loop insert
>SEQ ID NO: 152: WP_000861196 with Ena1B linker/HI-loop insert
>SEQ ID NO: 153: WP_086405534.1 with Ena1B linker/DE-loop insert
>SEQ ID NO: 154: WP_086405534.1 with Ena1B linker/HI-loop insert
>SEQ ID NO: 155: WP_142338290.1 with Ena1B linker/DE-loop insert
>SEQ ID NO: 156: WP_142338290.1 with Ena1B linker/HI-loop insert
>SEQ ID NO: 157: Sycon ciliatum diactinin (GenBank: SIP56239.1)
>SEQ ID NO: 158: Ena1B-HI-diactinin fusion
>SEQ ID NO: 159: R4.5-1RfD curlin-like repeat insert
>SEQ ID NO: 160: R4.5-2RfD curlin-like repeat insert
>SEQ ID NO: 161: R15.5-1R5 curlin-like repeat insert
>SEQ ID NO: 162: R15.5-2R5 curlin-like repeat insert
>SEQ ID NO: 163: R15.5-1R5fD curlin-like repeat insert
>SEQ ID NO: 164: R15.5-2R5fD curlin-like repeat insert
>SEQ ID NO: 165: Sycon ciliatum diactinin insert + linker/HI-loop
>SEQ ID NO: 166: R4.5-1RfD curlin-like repeat insert + linker/HI-loop
>SEQ ID NO: 167: R4.5-2RfD curlin-like repeat insert + linker/HI-loop
>SEQ ID NO: 168: R15.5-1R5 curlin-like repeat insert + linker/HI-loop
>SEQ ID NO: 169: R15.5-2R5 curlin-like repeat insert + linker/HI-loop
>SEQ ID NO: 170: R15.5-1R5fD curlin-like repeat insert + linker/HI-loop
>SEQ ID NO: 171: R15.5-2R5fD curlin-like repeat insert + linker/HI-loop
>SEQ ID NO: 172: Ena1B-HI-R4.5-1RfD fusion
>SEQ ID NO: 173: Ena1B-HI-R4.5-2RfD fusion
>SEQ ID NO: 174: Ena1B-HI-R15.5-1R5 fusion
>SEQ ID NO: 175: Ena1B-HI-R15.5-2R5 fusion
>SEQ ID NO: 176: Ena1B-HI-R15.5-1R5fD
>SEQ ID NO: 177: Ena1B-HI-R15.5-2R5fD
>SEQ ID NO: 178: Curlin repeat motif
>SEQ ID NO: 179: Curlin-like repeat motif
>SEQ ID NO: 180-184: linkers
>SEQ ID NO: 185: Ena1A-DE-Rubredoxin amino acid sequence (Ena1A (SEQ
ID NO: 1) with Rubredoxin (SEQ ID NO: 129, bold) inserted in the DE-
loop upon deletion of NQ and using G as linker (underlined))
MACECSSTVLTCCSDNSSNFVQDKVCNPWSSAEASTFTVYANNVNQNIVGTGYLTYDVGPGVSPAGKYVC
TVCGYEYDPAEGDPDNGVKPGTSFDDLPADWVCPVCGAPKSEFEAAGITVTVLDSGGGTIQTFLVNEGTS
ISFTFRRFNIIQITTPATPIGTYQGEFCITTRYLMA
>SEQ ID NO: 186: Ena1A (SEQ ID NO: 1) DE-loop sequence
>SEQ ID NO: 187: Ena1A (SEQ ID NO: 1) HI-loop sequence
>SEQ ID NO: 192: Cadmium binding 2MRB protein (Oryctolagus cuniculus)
>SEQ ID NO: 193: Zinc binding 1AQQ protein (Saccharomyces cerevisiae)
>SEQ ID NO: 194: Ena1B-HI-1AQQ amino acid sequence (Linkers
underlined. Insert in italics.)
MGNCSTNLSCCANGQKTIVQDKVCIDWTAAATAAIIYADNISQDIYASGYLKVDTGTGPVTIVFYSGGVT
GTAVETIVVATGSSASFTVRRFDTVTILGGGSGQNEGHECQCQCGSCKNNEQCQKSCSCPTGCNSDDKCP
CGNGAGGAAETGEFCMTIRYTLS
>SEQ ID NO: 195: Ena1B-2MRB-HI amino acid sequence (Linkers underlined.
Insert in italics.)
MGNCSTNLSCCANGQKTIVQDKVCIDWTAAATAAIIYADNISQDIYASGYLKVDTGTGPVTIVFYSGGVT
GTAVETIVVATGSSASFTVRRFDTVTILGGGSGMDPNCSCAAAGDSCTCANSCTCKACKCTSCKGAGGAA
ETGEFCMTIRYTLS
>SEQ ID NO: 196: Ena1B-DE_HAtag-HI-rubredoxin amino acid sequence (HA-
tag in bold. Linkers underlined. Inserts in italics.)
MGNCSTNLSCCANGQKTIVQDKVCIDWTAAATAAIIYADNISQDIYASGYLKVDTGSYPYDVPDYAGSGP
VTIVFYSGGVTGTAVETIVVATGSSASFTVRRFDTVTILGGGKYVCTVCGYEYDPAEGDPDNGVKPGTSF
DDLPADWVCPVCGAPKSEFEAAGAAETGEFCMTIRYTLS
>SEQ ID NO: 197: Ena1B-DE-rubredoxin (with arginine linker bold and
underlined)
MGNCSTNLSCCANGQKTIVQDKVCIDWTAAATAAIIYADNISQDIYASGYLKVDTGTGGKYVCTVCGYEY
DPAEGDPDNGVKPGTSFDDLPADWVCPVCGAPKSEFEAARVTIVFYSGGVTGTAVETIVVATGSSASFTV
RRFDTVTILGTAAAETGEFCMTIRYTLS
>SEQ ID NO: 198: Ena1B-DE-rubredoxin (with glycine linker bold and
underlined)
MGNCSTNLSCCANGQKTIVQDKVCIDWTAAATAAIIYADNISQDIYASGYLKVDTGTGGKYVCTVCGYEY
DPAEGDPDNGVKPGTSFDDLPADWVCPVCGAPKSEFEAAGVTIVFYSGGVTGTAVETIVVATGSSASFTV
RRFDTVTILGTAAAETGEFCMTIRYTLS

### REFERENCES

Aitken, C. E., Marshall, R. A., & Puglisi, J. D. (2008). An Oxygen Scavenging System for Improvement of Dye Stability in Single-Molecule Fluorescence Experiments. Biophysical Journal 94, 1826-1835.
Alberti, E., Consonni, R., & Zetta, L. (2003). Applications of NMR to Thermostable Proteins. Annual Reports on NMR Spectroscopy 50, 121-161.
Altamura, L., Horvath, C., Rengaraj, S., Rongier, A., Elouarzaki, K., Gondran, C., Maçon, A. L. B., Vendrely, C., Bouchiat, V., Fontecave, M., et al. (2017). A synthetic redox biofilm made from metalloprotein-prion domain chimera nanowires. Nature Chemistry 9, 157-163.
Ankolekar, C. & Labbé, R. G. (2010). Physical Characteristics of Spores of Food-Associated Isolates of the Bacillus cereus Group. Applied and Environmental Microbiology 76, 982-984.
Anonymous, AlphaFold Protein Structure Database. Accessed Jun. 1, 2022 https://alphafold.ebi.ac.uk/faq, consulted in June of 2022.
Antoine, E. E., Vlachos, P. P., & Rylander, M. N. (2014). Review of Collagen I Hydrogels for Bioengineered Tissue Microenvironments: Characterization of Mechanics, Structure, and Transport. Tissue Engineering Part B: Reviews 20, 683-696.
Aroul-Selvam, R., Hubbard, T., & Sasidharan, R. (2004). Domain Insertions in Protein Structures. Journal of Molecular Biology 338, 633-641.
Balbach, J. J., Ishii, Y., Antzutkin, O. N., Leapman, R. D., Rizzo, N. W., Dyda, F., Reed, J., & Tycko, R. (2000). Amyloid Fibril Formation by AB 16-22, a Seven-Residue Fragment of the Alzheimer's β-Amyloid Peptide, and Structural Characterization by Solid State NMR. Biochemistry 39, 13748-13759.
Bateman, A., Martin, M.-J., Orchard, S., Magrane, M., Agivetova, R., Ahmad, S., Alpi, E., Bowler-Barnett, E. H., Britto, R., Bursteinas, B., et al. (2021). UniProt: the universal protein knowledgebase in 2021. Nucleic Acids Research 49, D480-D489.
Beratan, D. N. & Skourtis, S. S. (2013). Electron Transfer Through Proteins. In: Encyclopedia of Biophysics (Roberts, C. K. G.), Springer Berlin Heidelberg, Berlin, Heidelberg, Germany, pp. 625-630.
Berman, H. M. (2000). The Protein Data Bank. Nucleic Acids Research 28, 235-242.
Bhatwa, A., Wang, W., Hassan, Y. I., Abraham, N., Li, X. Z., & Zhou, T. (2021). Challenges Associated With the Formation of Recombinant Protein Inclusion Bodies in Escherichia coli and Strategies to Address Them for Industrial Applications. Frontiers in Bioengineering and Biotechnology 9, 630551.
Bonomi, F., lametti, S., Kurtz, D. M., Richie, K. A., & Ragg, E. (1998). Direct metal ion substitution at the [M(SCys)4]2-site of rubredoxin. JBIC Journal of Biological Inorganic Chemistry 3, 595-605.
Bonomi, F., lametti, S., Mazzini, S., Fessas, D., & Kurtz, D. M. (2000). Thermal stability of Clostridium pasteurianum rubredoxin: Deconvoluting the contributions of the metal site and the protein. Protein Science 9, 2413-2426.
Botelho, H. M. & Gomes, C. M. (2011). Structural reorganization renders enhanced metalloprotein stability. Chemical Communications 47, 11149.
Bruscella, P., Cassagnaud, L., Ratouchniak, J., Brasseur, G., Lojou, E., Amils, R., & Bonnefoy, V. (2005). The HiPIP from the acidophilic Acidithiobacillus ferrooxidans is correctly processed and translocated in Escherichia coli, in spite of the periplasm pH difference between these two micro-organisms. Microbiology 151, 1421-1431.
Carter, C. W. Jr (2006). High Potential Iron Sulfur Proteins. In: Handbook of Metalloproteins (A. Messerschmidt, R. Huber, T. Poulas, K. Wieghardt, M. Cygler and W. Bode), John Wiley & Sons, Ltd, Chichester, West Sussex, England, 8 pp.
Cavaenero, S., Zhou, Z. H., Adams, M. W. W., & Chan, S. I. (1998). Unfolding mechanism of rubredoxin from Pyrococcus furiosus. Biochemistry 37, 3377-3385.
Cavagnero, S., Debe, D. A., Zhou, Z. H., Adams, M. W. W., Chan, S. I., & Amos, A. (1998). Kinetic Role of Electrostatic Interactions in the Unfolding of Hyperthermophilic and Mesophilic Rubredoxins. Biochemistry 37, 3369-3376.
Cavagnero, S., Zhou, Z. H., Adams, M. W. W., & Chan, S. I. (1995). Response of Rubredoxin from Pyrococcus furiosus to Environmental Changes: Implications for the Origin of Hyperthermostability. Biochemistry 34, 9865-9873.
Christen, R., Jancic, T., Zhou, Z. H., Adams, M. W. W., Tomich, J. M., & Smith, E. T. (1997). Physical characterization of a totally synthetic rubredoxin. Journal of Inorganic Biochemistry 65, 53-56.
Cleland, W. W. (1964). Dithiothreitol, a New Protective Reagent for SH Groups. Biochemistry 3, 480-482.
Cornelissen, R., Bøggild, A., Thiruvallur Eachambadi, R., Koning, R. I., Kremer, A., Hidalgo-Martinez, S., Zetsche, E.-M., Damgaard, L. R., Bonné, R., Drijkoningen, J., et al. (2018). The Cell Envelope Structure of Cable Bacteria. Frontiers in Microbiology 9, 3044.
Coulter, Eric D. & Kurtz, D. M. (2001). A Role for Rubredoxin in Oxidative Stress Protection in Desulfovibrio vulgaris: Catalytic Electron Transfer to Rubrerythrin and Two-Iron Superoxide Reductase. Archives of Biochemistry and Biophysics 394, 76-86.
Coustou, V., Deleu, C., Saupe, S., & Begueret, J. (1997). The protein product of the het-s heterokaryon incompatibility gene of the fungus Podospora anserina behaves as a prion analog. In: Proceedings of the National Academy of Sciences (D. D. Perkins), The National Academy of Sciences, Stanford, California, United State of America, pp. 9773-9778.
Dhami, M. K., Reddy, M. S. & Mukherjee, A. (2013) Biomineralization of calcium carbonates and their engineered applications: a review. Frontiers in Microbiology 4, 314.
Doble, M. & Kumar, A. (2005). Degradation of Dyes. In: Biotreatment of Industrial Effluents (M. Doble & A. Kumar), Elsevier, Oxford, Oxfordshire, United Kingdom, pp. 111-122.
Dorval Courchesne, N.-M., DeBenedictis, E. P., Tresback, J., Kim, J. J., Duraj-Thatte, A., Zanuy, D., Keten, S., & Joshi, N. S. (2018). Biomimetic engineering of conductive curli protein films. Nanotechnology 29, 454002.
Edwards, W. R., Busse, K., Allemann, R. K., & Jones, D. D. (2008). Linking the functions of unrelated proteins using a novel directed evolution domain insertion method. Nucleic Acids Research 36, e78.
Efimov, I., Parkin, G., Millett, E. S., Glenday, J., Chan, C. K., Weedon, H., Randhawa, H., Basran, J., & Raven, E. L. (2014). A simple method for the determination of reduction potentials in heme proteins. FEBS Letters 588, 701-704.
Eidsness, Marly K., Richie, K. A., Burden, A. E., Kurtz, D. M., & Scott, R. A. (1997). Dissecting contributions to the thermostability of Pyrococcus furiosus rubredoxin: beta-sheet chimeras. Biochemistry 36, 10406-10413.
Fauque, G. D., Moura, I., Moura, J. J. G., Xavier, A. v., Galliano, N., & LeGall, J. (1987). Isolation and characterization of a rubredoxin and a flavodoxin from Desulfovibrio desulfuricans Berre-Eau. FEBS Letters 215, 63-67.
Fowler, N. J. & Williamson, M. P. (2022). The accuracy of protein structures in solution determined by AlphaFold and NMR. Structure 30, 925-933.
Fukuyama, K. (2006). Ferredoxins Containing One [4Fe-4S] Center. In: Handbook of Metalloproteins (A. Messerschmidt, R. Huber, T. Poulas, K. Wieghardt, M. Cygler and W. Bode), John Wiley & Sons, Ltd, Chichester, West Sussex, England, 10 pp.
Geitner, A. J., Varga, E., Wehmer, M., & Schmid, F. X. (2013). Generation of a Highly Active Folding Enzyme by Combining a Parvulin-Type Prolyl Isomerase from SurA with an Unrelated Chaperone Domain. Journal of Molecular Biology 425, 4089-4098.
Giese, B. (2013). Electron Hopping in Biomolecules. In: Encyclopedia of Biophysics (Roberts, C. K. G.), Springer Berlin Heidelberg, Berlin, Heidelberg, Germany, pp. 586-588.
Goodfellow, B. J., Duarte, I. C. N., Macedo, A. L., Volkman, B. F., Nunes, S. G., Moura, I., Markley, J. L., & Moura, J. J. G. (2010). An NMR structural study of nickel-substituted rubredoxin. JBIC Journal of Biological Inorganic Chemistry 15, 409-420.
de Groot, N. S. & Ventura, S. (2006). Effect of temperature on protein quality in bacterial inclusion bodies. FEBS Letters 580, 6471-6476.
Gu, Y., Srikanth, V., Salazar-Morales, A. I., Jain, R., O'Brien, J. P., Yi, S. M., Soni, R. K., Samatey, F. A., Yalcin, S. E., & Malvankar, N. S. (2021). Structure of Geobacter pili reveals secretory rather than nanowire behaviour. Nature 597, 430-434.
Hachisuka, Y., Kozuka, S., & Tsujikawa, M. (1984). Exosporia and Appendages of Spores of Bacillus Species. Microbiology and Immunology 28, 619-624.
Hachisuka, Y. & Kuno, T. (1976). Filamentous Appendages of Bacillus cereus Spores. Japanese Journal of Microbiology 20, 555-558.
Handing, K. B., Niedzialkowska, E., Shabalin, I. G., Kuhn, M. L., Zheng, H., & Minor, W. (2018). Characterizing metal-binding sites in proteins with X-ray crystallography. Nature Protocols 13, 1062-1090.
Heinis, C. & Johnsson, K. (2010). Using Peptide Loop Insertion Mutagenesis for the Evolution of Proteins. In: In Vitro Mutagenesis Protocols (J. Braman), Humana Totowa, Stratagene, La Jolla, USA, pp. 217-232.
Higgitt, R. (2016). From cobwebs to silk: a world of human uses for spider thread. The Guardian.
Horneck, G., Moeller, R., Cadet, J., Douki, T., Mancinelli, R. L., Nicholson, W. L., Panitz, C., Rabbow, E., Rettberg, P., Spry, A., et al. (2012). Resistance of Bacterial Endospores to Outer Space for Planetary Protection Purposes—Experiment PROTECT of the EXPOSE-E Mission. Astrobiology 12, 445-456.
Hosseinzadeh, P. & Lu, Y. (2016). Design and fine-tuning redox potentials of metalloproteins involved in electron transfer in bioenergetics. Biochimica et Biophysica Acta (BBA)—Bioenergetics 1857, 557-581.
Hough, M. A. (2017). Choosing the optimal spectroscopic toolkit to understand protein function. Bioscience Reports 37, 4pp.
Ichiye, T. (2013). Electron Transfer Proteins: Overview. In: Encyclopedia of Biophysics (Roberts, C. K. G.), Springer Berlin Heidelberg, Berlin, Heidelberg, Germany, 13 pp.
Johnson, P. W. & Canale-Parola, E. (1973). Properties of rubredoxin and ferredoxin isolated from spirochetes. Archiv for Mikrobiologie 89, 341-353.
Jumper, J., Evans, R., Pritzel, A., Green, T., Figurnov, M., Ronneberger, O., Tunyasuvunakool, K., Bates, R., Žídek, A., Potapenko, A., et al. (2021). Highly accurate protein structure prediction with AlphaFold. Nature 596, 583-589.
Kalyoncu, E., Ahan, R. E., Olmez, T. T., & Safak Seker, U. O. (2017). Genetically encoded conductive protein nanofibers secreted by engineered cells. RSC Advances 7, 32543-32551.
Khan, F. A. (2020). Synthesis of Nanomaterials: Methods & Technology. In: Applications of Nanomaterials in Human Health. (F. A Khan), Springer Singapore, Singapore, Singapore, pp. 15-21.
Kim, C.-S., Pierre, B., Ostermeier, M., Looger, L. L., & Kim, J. R. (2009). Enzyme stabilization by domain insertion into a thermophilic protein. Protein Engineering Design and Selection 22, 615-623.
Kitamura, M., Koshino, Y., Kamikawa, Y., Kohno, K., Kojima, S., Miura, K. I., Sagara, T., Akutsu, H., Kumagai, I., & Nakaya, T. (1997). Cloning and expression of the rubredoxin gene from Desulfovibrio vulgaris (Miyazaki F)—comparison of the primary structure of desulfoferrodoxin. Biochimica et Biophysica Acta—Gene Structure and Expression 1351, 239-247.
Krieger, F., Möglich, A., & Kiefhaber, T. (2005). Effect of proline and glycine residues on dynamics and barriers of loop formation in polypeptide chains. Journal of the American Chemical Society 127, 3346-3352.
Kumar, A., Choudhary, P., Kumar, A., Camargo, P. H., Krishnan, V., (2022). Recent ad-vances in plasmonic photocatalysis based on TiO2 and noble metal nanoparticles for energy conversion, environmental remediation, and organic synthesis. Small 18 (1), p.2101638.
Lewis, R. (1996). Unraveling the Weave of Spider Silk. BioScience 46, 636-638.
Li, Y., Sierra, A. M., Ai, H., & Campbell, R. E. (2007). Identification of Sites Within a Monomeric Red Fluorescent Protein that Tolerate Peptide Insertion and Testing of Corresponding Circular Permutations. Photochemistry and Photobiology 84, 111-119.
Liu, J., Chakraborty, S., Hosseinzadeh, P., Yu, Y., Tian, S., Petrik, I., Bhagi, A., & Lu, Y. (2014). Metalloproteins Containing Cytochrome, Iron-Sulfur, or Copper Redox Centers. Chemical Reviews 114, 4366-4469.
Logan, N. A. & de Vos, P. (2015). Bacillus. Bergey's Manual of Systematics of Archaea and Bacteria, 1-163.
Lombardi, L., Falanga A., Del Genio V., and Galdiero S. A New hope: self-assembling peptides with antimicrobial activity. Pharmaceutics 2019, 11, 166.
Lovenberg, W. (1974). FERREDOXIN AND RUBREDOXIN. In: Microbial Iron Metabolism (Neilands J.), Elsevier, New York, New York, USA, pp. 161-185.
Lovley, D. R. (2022). On the Existence of Pilin-Based Microbial Nanowires. Frontiers in Microbiology 13, 5 pp.
Lovley, D. R. & Yao, J. (2021). Intrinsically Conductive Microbial Nanowires for ‘Green’ Electronics with Novel Functions. Trends in Biotechnology 39, 940-952.
Lukaszczyk, M., Pradhan, B., & Remaut, H. (2019). The Biosynthesis and Structures of Bacterial Pili. In: Bacterial Cell Walls and Membranes (Kuhn, A.), Springer Nature, Zurich, Switzerland, pp. 369-413.
Maher, M., Cross, M., Wilce, M. C. J., Guss, J. M., & Wedd, A. G. (2004). Metal-substituted derivatives of the rubredoxin from Clostridium pasteurianum. Acta Crystallographica Section D Biological Crystallography 60, 298-303.
Malvankar, N. S. & Lovley, D. R. (2014). Microbial nanowires for bioenergy applications. Current Opinion in Biotechnology 27, 88-95.
Mathieu, I., Meyer, J., & Moulis, J. M. (1992). Cloning, sequencing and expression in Escherichia coli of the rubredoxin gene from Clostridium pasteurianum. Biochemical Journal 285, 255-262.
May, S. W. & Kuo, J. Y. (1977). Preparation and properties of immobilized rubredoxin. Journal of Biological Chemistry 252, 2390-2395.
MAYHEW, S. G. (1978). The Redox Potential of Dithionite and SO-2 from Equilibrium Reactions with Flavodoxins, Methyl Viologen and Hydrogen plus Hydrogenase. European Journal of Biochemistry 85, 535-547.
Messerschmidt, A., Huber, R., Poulas, T., Wieghardt, K., Cygler, M., & Bode, W. eds. (2006). Handbook of Metalloproteins, John Wiley & Sons, Ltd, Chichester, West Sussex, England, 2448 pp.
Meyer, J. & Moulis, J.-M. (2006). Rubredoxin. In: Handbook of Metalloproteins (A. Messerschmidt, R. Huber, T. Poulas, K. Wieghardt, M. Cygler and W. Bode), John Wiley & Sons, Ltd, Chichester, West Sussex, England, 13 pp.
Meysman, F. J. R., Cornelissen, R., Trashin, S., Bonné, R., Martinez, S. H., van der Veen, J., Blom, C. J., Karman, C., Hou, J.-L., Eachambadi, R. T., et al. (2019). A highly conductive fibre network enables centimetre-scale electron transport in multicellular cable bacteria. Nature Communications 10, 4120.
Miotto, M., Olimpieri, P. P., Rienzo, L. di, Ambrosetti, F., Corsi, P., Lepore, R., Gaetano Tartaglia, G., & Milanetti, E. Insights on protein thermal stability: a graph representation of molecular interactions. Bioinformatics 35, 2569-2577.
Mizuki, E., Ohba, M., Ichimatsu, T., Hwang, S.-H., Higuchi, K., Saitoh, H., & Akao, T. (1998). Unique appendages associated with spores of Bacillus cereus isolates. Journal of Basic Microbiology 38, 33-39.
Moir, A. & Cooper, G. (2015). Spore Germination. Microbiology Spectrum 3, 14 pp.
Moura, I., Teixeira, M., Moura, J. J. G., & LeGall, J. (1991). Spectroscopic studies of cobalt and nickel substituted rubredoxin and desulforedoxin. Journal of Inorganic Biochemistry 44, 127-139.
Moura, J. J., Macedo, A. L., Goodfellow, B. J., & Moura, I. (2006). Ferredoxins Containing One [3Fe-4S] Cluster. Desulfovibrio Gigas Ferredoxin II—Solution Structure. In: Handbook of Metalloproteins (A. Messerschmidt, R. Huber, T. Poulas, K. Wieghardt, M. Cygler and W. Bode), John Wiley & Sons, Ltd, Chichester, West Sussex, England, 7 pp.
Nguyen, P. Q., Botyanszki, Z., Tay, P. K. R., & Joshi, N. S. (2014). Programmable biofilm-based materials from engineered curli nanofibres. Nature Communications 5.
Noy, D., Moser, C. C., & Dutton, P. L. (2006). Design and engineering of photosynthetic light-harvesting and electron transfer using length, time, and energy scales. Biochimica et Biophysica Acta (BBA)-Bioenergetics 1757, 90-105.
Ohno, H., Takeda, K., Niwa, S., Tsujinaka, T., Hanazono, Y., Hirano, Y., & Miki, K. (2017). Crystallographic characterization of the high-potential iron-sulfur protein in the oxidized state at 0.8 Å resolution. PLOS ONE 12, e0178183.
Pais, T. M., Lamosa, P., dos Santos, W., LeGall, J., Turner, D. L., & Santos, H. (2005). Structural determinants of protein stabilization by solutes. FEBS Journal 272, 999-1011.
Papavassiliou, P. & Hatchikian, E. C. (1985). Isolation and characterization of a rubredoxin and a two-(4Fe-4S) ferredoxin from Thermodesulfobacterium commune. Biochimica et Biophysica Acta (BBA)-Bioenergetics 810, 1-11.
Pfeffer, C., Larsen, S., Song, J., Dong, M., Besenbacher, F., Meyer, R. L., Kjeldsen, K. U., Schreiber, L., Gorby, Y. A., El-Naggar, M. Y., et al. (2012). Filamentous bacteria transport electrons over centimetre distances. Nature 491, 218-221.
Piet De Vries, Y. (2006). Bacillus cereus spore formation, structure, and germination, Wageningen Universiteit, Wageningen, Gelderland, The Netherlands, 113 pp.
Pradhan, B., Liedtke, J., Sleutel, M., Lindbäck, T., Zegeye, E. D., O'Sullivan, K., Llarena, A.-K., Brynildsrud, O., Aspholm, M., & Remaut, H. (2021). Endospore Appendages: a novel pilus superfamily from the endospores of pathogenic Bacilli. The EMBO Journal 40, e106887.
Qing, G., Ma, L.-C., Khorchid, A., Swapna, G. V. T., Mal, T. K., Takayama, M. M., Xia, B., Phadtare, S., Ke, H., Acton, T., et al. (2004). Cold-shock induced high-yield protein production in Escherichia coli. Nature Biotechnology 22, 877-882.
Rajasekar, et al., (2021). MICP as a potential sustainable technique to treat or entrap contaminants in the natural environment: A review, Environmental Science and Ecotechnology, 6, 100096.
Remaut, H. & Waksman, G. (2006). Protein-protein interaction through β-strand addition. Trends in Biochemical Sciences 31, 436-444.
Rode, L. J. & Slepecky, R. A. (1971). Bacterial Spore Appendages. CRC Critical Reviews in Microbiology 1, 1-27.
Rode, L. J. & Smith, L. D. (1971). Taxonomic Implications of Spore Fine Structure in Clostridium bifermentans. Journal of Bacteriology 105, 349-354.
Sampaio, R. N., Piechota, E. J., Troian-Gautier, L., Maurer, A. B., Hu, K., Schauer, P. A., Blair, A. D., Berlinguette, C. P., & Meyer, G. J. (2018). Kinetics teach that electronic coupling lowers the free-energy change that accompanies electron transfer. Proceedings of the National Academy of Sciences 115, 7248-7253.
Sánchez, B., Arias, S., Chaignepain, S., Denayrolles, M., Schmitter, J. M., Bressollier, P., & Urdaci, M. C. (2009). Identification of surface proteins involved in the adhesion of a probiotic Bacillus cereus strain to mucin and fibronectin. Microbiology 155, 1708-1716.
Scalley-Kim, M., Minard, P., & Baker, D. (2003). Low free energy cost of very long loop insertions in proteins. Protein science 12, 197-206.
Seker, U. O. S., Chen, A. Y., Citorik, R. J., & Lu, T. K. (2017). Synthetic Biogenesis of Bacterial Amyloid Nanomaterials with Tunable Inorganic-Organic Interfaces and Electrical Conductivity. ACS Synthetic Biology 6, 266-275.
Senthil, R., Usha, S., & Saravanan, K. M. (2019). Importance of Fluctuating Amino Acid Residues in Folding and Binding of Proteins. Avicenna journal of medical biotechnology 11, 339-343.
Shapiro, D. M., Mandava, G., Yalcin, S. E., Arranz-Gibert, P., Dahl, P. J., Shipps, C., Gu, Y., Srikanth, V., Salazar-Morales, A. I., O'Brien, J. P., et al. (2022). Protein nanowires with tunable functionality and programmable self-assembly using sequence-controlled synthesis. Nature Communications 13, 10 pp.
Singh, A., Upadhyay, V., Upadhyay, A. K., Singh, S. M., & Panda, A. K. (2015). Protein recovery from inclusion bodies of Escherichia coli using mild solubilization process. Microbial Cell Factories 14, 41.
Slater, J. W., Marguet, S. C., Monaco, H. A., & Shafaat, H. S. (2018). Going beyond Structure: Nickel-Substituted Rubredoxin as a Mechanistic Model for the [NiFe] Hydrogenases. Journal of the American Chemical Society 140, 10250-10262.
Sleutel, M., Pradhan, B., & Remaut, H. (2022). Structural analysis of the bacterial amyloid curli. bioRxiv, 1-40.
STATES, D. J. & GISH, W. (1994). QGB: Combined Use of Sequence Similarity and Codon Bias for Coding Region Identification. Journal of Computational Biology 1, 39-50.
Strodel, B. (2021). Energy Landscapes of Protein Aggregation and Conformation Switching in Intrinsically Disordered Proteins. Journal of Molecular Biology 433, 167182.
Sushko, T., Kavaleuski, A., Grabovec, I., Kavaleuskaya, A., Vakhrameev, D., Bukhdruker, S., Marin, E., Kuzikov, A., Masamrekh, R., Shumyantseva, V., et al. (2021). A new twist of rubredoxin function in M. tuberculosis. Bioorganic Chemistry 109, 104721.
Teske, A. (2019). Cable bacteria, living electrical conduits in the microbial world. Proceedings of the National Academy of Sciences 116, 18759-18761.
Topcu, G., et al (2019). Ionic Polymer and Metal Composites Thereof. In: Ionic Polymer Metal Composites for Sensors and Actuators (Inamuddin & Asiri, A. M.), Springer International Publishing, Cham, Zug, Switzerland, pp. 171-171.
Tunyasuvunakool, K., Adler, J., Wu, Z. et al. (2021). Highly accurate protein structure prediction for the human proteome. Nature 596, 590-596.
Ueki, T., Walker, D. J. F., Tremblay, P.-L., Nevin, K. P., Ward, J. E., Woodard, T. L., Nonnenmann, S. S., & Lovley, D. R. (2019). Decorating the Outer Surface of Microbially Produced Protein Nanowires with Peptides. ACS Synthetic Biology 8, 1809-1817.
Varadi, et al. (2022). Alphafold Protein Structure Database: massively expanding the structural coverage of protein-sequence space with high-accuracy models, Nucleic Acids Research, 50, D1, p. D439-D444.
Walker, J. R., Gnanam, A. J., Blinkova, A. L., Hermandson, M. J., Karymov, M. A., Lyubchenko, Y. L., Graves, P. R., Haystead, T. A., & Linse, K. D. (2007). Clostridium taeniosporum spore ribbon-like appendage structure, composition and genes. Molecular Microbiology 63, 629-643.
Wenger, O. S. (2011). Photoinduced electron and energy transfer in phenylene oligomers. Chemical Society Reviews 40, 3538.
Whittall, D. R., Baker, K. v., Breitling, R., & Takano, E. (2021). Host Systems for the Production of Recombinant Spider Silk. Trends in Biotechnology 39, 560-573.
Wijnands, L. M., Dufrenne, J. B., van Leusden, F. M., & Abee, T. (2007). Germination of Bacillus cereus Spores Is Induced by Germinants from Differentiated Caco-2 Cells, a Human Cell Line Mimicking the EPithelial Cells of the Small Intestine. Applied and Environmental Microbiology 73, 5052-5054.
Yalcin, S. E. & Malvankar, N. S. (2020). The blind men and the filament: Understanding structures and functions of microbial nanowires. Current Opinion in Chemical Biology 59, 193-201.
Yan, B. & Smith, J. W. (2001). Mechanism of Integrin Activation by Disulfide Bond Reduction. Biochemistry 40, 8861-8867.
Zanetti, G. & Pandini, V. (2013). Ferredoxin. In: Encyclopedia of Biological Chemistry (M. D. Lane), Elsevier London, Greater London, United Kingdom, pp. 296-298.
Zegeye, E. D., Pradhan, B., Llarena, A.-K., & Aspholm, M. (2021). Enigmatic Pilus-Like Endospore Appendages of Bacillus cereus Group Species. International Journal of Molecular Sciences 22, 12367.
Zoldák, G., Carstensen, L., Scholz, C., & Schmid, F. X. (2009). Consequences of Domain Insertion on the Stability and Folding Mechanism of a Protein. Journal of Molecular Biology 386, 1138-1152.
Zhu, & Dittrich (2016). Carbonate precipitation through microbial activities in natural environment, and their potential in biotechnology: a review, Front. Microbiol. 4, 1e21.

1. A self-assembling endospore appendage (Ena) fusion protein comprising:
a metal-binding polypeptide (MBP) fused to an Ena protein,
wherein the MBP is comprised in an insert that is fused fusion is made via insertion of said MBP into a loop region exposed on the Ena protein surface, wherein the insertion is made directly or via a linker.

2. The Ena fusion protein according to of claim 1, wherein the Ena protein is selected from the proteins presented by group consisting of any one of SEQ ID NOs: 1-82, a homologue with at least 80% identity of any one thereof, and an engineered Ena protein of any one thereof.

3. The Ena fusion protein according to any one of claim 1 or 2 of claim 1, wherein the insertion of the MBP, optionally containing a linker, is made via fusing the insert is fused to the N- and C-terminal end of an interrupted loop on the (engineered) Ena protein surface, preferably wherein the interrupted loop of the Ena protein surface is loop DE or HI.

4. The Ena fusion protein according to any one of claims 1 to 3 of claim 1, wherein the MBP specifically binds any one of the metals Be, Mg, Ca, Sr, Ba, Ra, Sc, Ti, V, Cr, Mn, Fe, Co, Ni, Cu, Zn, Y, Zr, Nb, Mo, Tc, Ru, Rh, Pd, Ag, Cd, Lu, Hf, Ta, W, Re, Os, Ir, Pt, Au, Hg, Se, Ag, As, Pb and/or salts thereof, and/or comprises a MBP classified as iron-sulphur proteins, high potential iron-sulfur proteins (HiPIPs), or metallothionein.

5. The Ena fusion protein according to any one of claims 1 to 4 of claim 1, wherein the insertion is made via genetic fusion of an MBP within the Ena protein, preferably wherein the Ena fusion protein comprises an Ena protein lacking loop DE, or lacking loop HI, or wherein the loop DE or HI may be is truncated by a maximum of four amino acids as compared to the wild type Ena protein, and/or wherein inserted MBP may contain contains additional linker amino acids N- and/or C-terminally fused to the MBP.

6. The Ena fusion protein of claim 1, wherein the ENA fusion protein is comprised in a multimer, the multimer comprising at least two Ena proteins wherein at least one Ena protein is the Ena fusion protein according to any one of claims 1 to 5 claim 1, and wherein said the Ena (fusion) proteins are present in the multimer as non-covalently linked subunits.

7. The Ena fusion protein of claim 6, wherein the multimer is present in a protein fiber comprising at least 2 of the multimers according to claim 6, and wherein said the multimers are longitudinally stacked and covalently linked through at least one disulphide bond.

8. A chimeric gene comprising a nucleic acid molecule encoding the The Ena fusion protein according to any one of claims 1 to 5 of claim 1, wherein the Ena fusion protein is produced by expression of a nucleic acid encoding the Ena fusion protein.

9. The Ena fusion protein of claim 1, wherein the Ena fusion protein is comprised in a host cell comprising the Ena fusion protein of any one of claims 1 to 5, the multimers of claim 6, the protein fiber of claim 7, and/or the chimeric gene of claim 8.

10. A method for recombinant production of the Ena fusion protein of claim 1, multimer, or fiber, according to any one of claims 1 to 7, the method comprising the steps of:
a. Expression of the chimeric gene of claim 8 expressing the Ena fusion protein in a host cell, or cultivating the host cell of claim 9, and
b. Isolation of isolating the Ena fusion proteins protein as monomers, multimers, or fibers from said cultivated cell culture the host cell and/or cultivation medium thereof.

11. The method according to claim 10, wherein the isolation in step b. comprises the steps of:
1. mechanical or chemical cell lysis lysing and/or enzymological digestion digesting of undesired host cell polymers, preferably by glycosylhydrolases, proteases or nucleases, and/or
2. incubation of incubating the cell lysate and/or cultivation medium in a heated denaturing solution, and
3. recovery of recovering the Ena fusion protein, multimer, or fiber by sedimentation or ultrafiltration.

12. A computer-implemented method for structure-guided identification of a polypeptide or fragment thereof as insert to produce an Ena fusion protein, the method comprising the steps of:
a. Predict the predicting a tridimensional structure of one or more Ena protein polypeptide sequences on a computer by running with a program which predicts capable to predict 3D-protein structures, such as the Alphafold 2.0 program,
b. From from the predicted structures, select selecting those polypeptides, or fragments thereof, for which the N- and C-terminus terminals are at a distance from each other that is suitable to insert within the Ena protein at a lacking exposed loop on the Ena surface of an Ena protein,
wherein a suitable distance means that is where the N- and C-terminals of said the Ena protein can be genetically fused via the insert to the loop for insertion, optionally via short linker residues, to bridge the distance of said N- and C-terminals, and
c. optionally, optimize the length of the short linker for fiber formation upon self-assembly by linker shuffling; and producing a nucleic acid encoding the insert.

13. The computer-implemented method according to claim 12, further comprising identifying putative metal-binding polypeptides as those polypeptide structures with 3, 4, or more free, non-disulfide bonded, cysteines in close proximity.

14. The computer-implemented method according to claim 12, wherein in step b, those selecting polypeptides or fragments thereof comprises selecting polypeptide or fragments thereof where the N- and C-terminus are at a distance from each other that is suitable to insert within the Ena protein at a lacking DE- or HI-loop, and wherein a suitable distance is where the N- and C-term can be genetically fused to the loop to form an insertion, with or without a short linker, to bridge a distance of 10±2 angstroms.

15. (canceled)

16. (canceled)

17. (canceled)

18. (canceled)

19. The Ena fusion protein of claim 1, wherein the insertion is made via a linker.

20. The Ena fusion protein of claim 3, wherein the interrupted loop of the Ena protein surface is loop DE or HI.

21. The method according to claim 11, wherein the undesired host cell polymers, are digested by glycosylhydrolases, proteases, and/or nucleases.

22. The method according to claim 12, wherein the insert comprises a short linker.

23. The method according to claim 22, further comprising optimizing the length of the short linker for fiber formation upon self-assembly.

24. The method according to claim 23, wherein the optimization is performed by linker shuffling.
